SEQUENCE LISTING (4 sequences)

SEQ ID NO: 1 - protein, 188 aa, Artificial
  Synthetic protein
MASSHHHHHHSSENLYFQGMTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 2 - protein, 487 aa, Artificial
  Synthetic protein
GEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

SEQ ID NO: 3 - DNA, 567 bp, Artificial
  Synthetic DNA
atggcaagcagccatcatcatcatcatcatagcagcgaaaacctgtattttcagggcatgaccgaatataaactggttgttgttggtgcagatggtgttggtaaaagcgcactgaccattcagctgattcagaatcattttgtggatgagtatgatccgaccatcgaagatagctatcgtaaacaggttgtgattgatggtgaaacctgtctgctggatattctggataccgcaggtcaagaggaatatagcgcaatgcgtgatcagtatatgcgtaccggtgaaggttttctgtgtgtttttgcaatcaacaataccaaaagcttcgaggatatccatcattatcgcgagcagattaaacgtgtgaaagatagcgaagatgttccgatggttctggttggtaataaatgtgatctgccgagccgtaccgttgataccaaacaggcacaggatctggcacgtagctatggtattccgtttattgaaaccagcgcaaaaacccgtcagggtgttgatgatgcattttataccctggttcgtgaaatccgcaaacataaagaaaaatga

SEQ ID NO: 4 - DNA, 1584 bp, Artificial
  Synthetic DNA
atgggcagcagccatcatcatcatcatcacagcagcggcctggtgccgcgcggcagccatatggctagcatgactggtggacagcaaatgggtcgcggatccgaaaacctgtattttcagggcgaggagcagatgaggctgcctagtgctgatgtttatagatttgcagagcctgactctgaagagaatattatatttgaagagaacatgcagcccaaggctggaattccaattatcaaagcaggaactgttattaaacttatagagaggcttacgtaccatatgtacgcagatcccaattttgttcggacatttcttacaacatacagatccttttgcaaacctcaagaactactgagtcttataatagaaaggtttgaaattccagagcctgagccaacagaagctgatcgcatagctatagagaatggagatcaacccttgagtgcagaactgaaaagatttagaaaagaatatatacagcctgtgcaactgcgagtattaaatgtatgtcggcactgggtagagcaccacttctatgattttgaaagagatgcatatcttttgcaacgaatggaagaatttattggaacagtaagaggtaaagcaatgaaaaaatgggttgaatccatcactaaaataatccaaaggaaaaaaattgcaagagacaatggaccaggtcataatattacatttcagagttcacctcccacagttgagtggcatataagcagacctgggcacatagagacttttgacctgctcaccttacacccaatagaaattgctcgacaactcactttacttgaatcagatctataccgagctgtacagccatcagaattagttggaagtgtgtggacaaaagaagacaaagaaattaactctcctaatcttctgaaaatgattcgacataccaccaacctcactctgtggtttgagaaatgtattgtagaaactgaaaatttagaagaaagagtagctgtggtgagtcgaattattgagattctacaagtctttcaagagttgaacaactttaatggtgtccttgaggttgtcagtgctatgaattcatcacctgtttacagactagaccacacatttgagcaaataccaagtcgccagaagaaaattttagaagaagctcatgaattgagtgaagatcactataagaaatatttggcaaaactcaggtctattaatccaccatgtgtgcctttctttggaatttatctcactaatatcttgaaaacagaagaaggcaaccctgaggtcctaaaaagacatggaaaagagcttataaactttagcaaaaggaggaaagtagcagaaataacaggagagatccagcagtaccaaaatcagccttactgtttacgagtagaatcagatatcaaaaggttctttgaaaacttgaatccgatgggaaatagcatggagaaggaatttacagattatcttttcaacaaatccctagaaatagaaccacgaaaccctaagcctctcccaagatttccaaaaaaatatagctatcccctaaaatctcctggtgttcgtccatcaaacccaagaccaggtacctaa

(19) United States
(12) Patent Application Publication
(10) Pub. No.: US 2023/0348495 A1
(43) Pub. Date: Nov. 2, 2023

(54) A COMPOUND HAVING INHIBITORY ACTIVITY AGAINST KRAS G12D MUTATION

(71) Applicants: TAIHO PHARMACEUTICAL CO., LTD., Tokyo (JP); ASTEX THERAPEUTICS, LTD., Cambridge (GB)

(72) Inventors: YUICHI KAWAI, Ibaraki (JP); KAZUAKI SHIBATA, Ibaraki (JP); HIROKI ASAKURA, Ibaraki (JP); TAKAO UNO, Ibaraki (JP); TAKESHI SAGARA, Ibaraki (JP); MASAYUKI NAKAMURA, Ibaraki (JP); YU KOBAYAKAWA, Ibaraki (JP); RHIAN SARA HOLVEY, Cambridge (GB)

(21) Appl. No.: 17/780,597

(22) PCT Filed: Nov. 27, 2020
(86) PCT No.: PCT/JP2020/045146

§ 371 Date: May 27, 2022

(30) Foreign Application Priority Data
Nov. 29, 2019 (JP) 2019-216165
Dec. 6, 2019 (WO) PCT/JP2019/049074

(51) Int. Cl. C07D 519/00; C07D 487/08; A61P 35/00; A61K 45/06

(52) U.S. Cl. CPC C07D 519/00; C07D 487/08; A61P 35/00; A61K 45/06

(57) ABSTRACT

The present invention relates to a compound having inhibitory activity against KRAS G12D mutation or a salt thereof, and relates to a pharmaceutical composition comprising the compound as an active ingredient.

### TECHNICAL FIELD

The present invention relates to a compound having inhibitory activity against KRAS G12D mutation or a salt thereof, and relates to a pharmaceutical composition comprising the compound as an active ingredient.

### BACKGROUND ART

RAS, which is a small monomeric GTP-binding protein having a molecular weight of about 21 kDa, acts as a molecular on/off switch. RAS can bind to GTP by binding to proteins of a guanine nucleotide exchange factor (GEF) (e.g., SOS1), which forces the release of a bound nucleotide, and releasing GDP. When RAS binds to GTP, it becomes activated type (turned on), and recruits and activates proteins necessary for the propagation of other receptors' signals, such as c-Raf and PI 3-kinase. RAS also possesses enzymatic activity with which it cleaves the terminal phosphate of the nucleotide and converts it to GDP. The rate of conversion is usually slow, but can be dramatically sped up by a protein of the GTPase-activating protein (GAP) class, such as RasGAP. When GTP is converted into GDP, RAS is deactivated (turned off).

The mainly known members of the RAS subfamily include HRAS, KRAS, and NRAS. Of these, mutations of KRAS are observed in many malignant tumors: 95% of pancreatic ductal adenocarcinomas (PDAC), 45% of colon and rectal carcinomas (CRC), and 35% of non-small cell lung carcinomas (NSCLC). The mutations often occur in the glycine residue of KRAS at position 12 in 82% of PDAC, 64% of CRC, 92% of NSCLC. Among such mutations, the predominant mutation of KRAS at position 12 in PDAC (39%) and CRC (44%) has been reported to be a mutation into aspartic acid (Non-patent Literature (NPL) 1).

RAS was considered to be undruggable for many years. However, it has been reported that targeting the inactive, GDP bound KRAS (G12C) is promising approach for generating novel anti-RAS therapies (NPL 2). Because KRAS (G12C) retains the GTPase activity and the nucleotide cycling exist in KRAS (G12C) cell, an inhibitor bound to inactive KRAS (G12C) can inhibit the activation of KRAS (G12C) in cells. As well as KRAS (G12C) mutant, it has been reported that KRAS (G12D) also retain GTPase activity (NPL 3). Therefore the strategy for targeting the GDP-bound KRAS (G12D) and inhibiting the conversion from GDP to GTP bound state is thought to be extremely attractive.

### CITATION LIST

### Non-Patent Literature

NPL 1: Nature Reviews Drug Discovery 13 (11), 828-51, 2014
NPL 2: Cancer Discov. 6 (3), 316-29, 2016
NPL 3: Mol Cancer Res; 13(9), 1325-35, 2015

### SUMMARY OF INVENTION

### Problem to be Solved by the Invention

An object of the present invention is to provide a novel compound or a salt thereof that inhibits function of KRAS G12D mutant, and to provide a pharmaceutical composition comprising the compound.

The present inventors conducted extensive research to solve the above problems, and consequently found that the group of compounds represented by the following formula (1), strongly inhibits functions of KRAS. The present invention has thus been accomplished.

More specifically, the present invention provides the following [1] to [36].

[1] A compound represented by the formula (1) or a salt thereof:

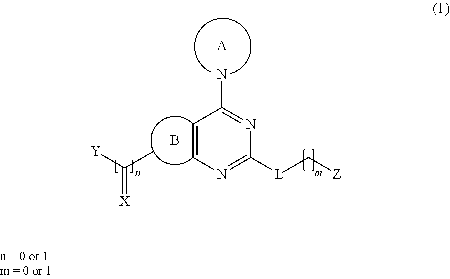

wherein
Ring A represents a substituted or unsubstituted, saturated or unsaturated 8-to 10-membered N-containing bridged ring which contains at least one further heteroatom selected from the group consisting of N, S and O;
Ring B represents a substituted or unsubstituted, 5- to 6-membered saturated or unsaturated ring having at least one heteroatom selected from the group consisting of N, S, and O, a 6-membered aromatic hydrocarbon ring, C3-C6 cycloalkyl ring, C3-C6 cycloalkenyl or an 8- to 10-membered spiro ring, wherein the Ring B is fused with the pyrimidine ring to form a substituted or unsubstituted bicyclic ring;
n is 0 or 1;
X is O or S;
Y represents a substituted or unsubstituted, 6- to 10-membered unsaturated monocyclic or bicyclic ring which contains at least one heteroatom selected from the group consisting of N, S and O, or 6- to 10-membered aromatic hydrocarbon ring;
L represents oxygen atom, or a substituted or unsubstituted C2-C3 alkynyl;
Z represents cyanoalkyl, alkylcarbonylaminoalkyl, alkylaminocarbonyl, alkylaminoalkyl, a substituted or unsubstituted, C3-C6 cycloalkyl, a 5- to 6-membered saturated ring which contains at least one heteroatom selected from the group consisting of N, S and O, or an 8-to 10-membered partially unsaturated ring which contains at least one heteroatom selected from the group consisting of N, S and O;
when L is C2-C3 alkynyl, Z is alkylaminocarbonyl or alkylaminoalkyl;
m is 0 or 1.

[2] The compound or salt thereof according to [1], wherein the 8- to 10-membered N-containing bridged ring is piperazinyl ring-based, 8-membered N-containing bridged ring, which may be substituted by R1 or R2, and when the 8-membered N-containing bridged ring is substituted by R1, the R1 is substituted on a nitro atom of piperazinyl ring, and is substituted by R2, the R2 is substituted on any one of carbon atoms of piperazinyl ring; wherein the R1 represents hydrogen atom, or hydroxyl, and the R2 represents a hydrogen atom, halogen atom, alkoxycarbonyl, cyano, or hydroxyalkyl.

[3] The compound or salt thereof according to [1] or [2], wherein the Ring A is represented by any one of the formula (2a) to (2c) which may be substituted by R1 and R2:

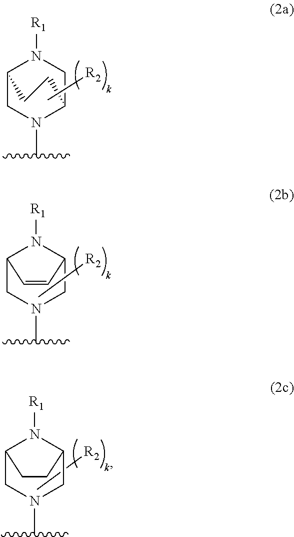

wherein R1 represents hydrogen atom, C1-C6 alkyl, or hydroxyl; R2 represents hydrogen atom, halogen atom, alkoxycarbonyl, cyano, or hydroxyalkyl; and
k is 0 to 6.

[4] The compound or salt thereof according to any one of [1] to [3],
wherein the Ring A is represented by the formula (3a) or (3b):

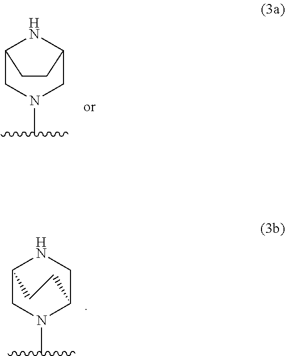 [SMILES: *N1CC2CCC(C1)N2 ; *N1C[C@@H]2CC[C@H]1CN2]

[5] The compound or salt thereof according to any one of [1] to [4],
wherein the Ring B represents:
(i) a 5- to 6-membered saturated or unsaturated ring which contains at least one heteroatom selected from the group consisting of N, S, and O,
(ii) a 6- to 10-membered aromatic hydrocarbon ring,
(iii) C3-C6 cycloalkyl, C3-C6 cyclealkenyl or
(iv) an 8- to 10-membered spiro ring;
wherein the Ring B is fused with the pyrimidine ring to form a substituted or unsubstituted bicyclic ring; and
wherein the Ring B in the bicyclic ring may be substituted by halogen atom, C1-C6 alkyl, alkylcarbonyl or 4- to 6-membered saturated monocyclic ring which contains at least one heteroatom selected from N, S, and O.

[6] The compound or salt thereof according to [5], wherein the heteroatom in 5- to 6-membered saturated or unsaturated ring is N or O.

[7] The compound or salt thereof according to any one of [1] to [6],
wherein the Ring B represents benzene, piperidine, pyrrolidine, cyclohexane, cyclohexene, tetrahydro-2H-pyran, 3,4-dihydro-2H-pyran, or spiro[2.5]octane;
wherein the Ring B may be substituted by halogen atom, C1-C6 alkyl, alkylcarbonyl or oxetanyl; and
when the Ring B is pyrrolidine, n is 1 and X is O or S, and
when the Ring B is not pyrrolidine, n is 0.

[8] The compound or salt thereof according to any one of [1] to [7],
wherein the Ring B represents unsubstituted benzene, piperidine, pyrrolidine, tetrahydro-2H-pyran or 3,4-dihydro-2H-pyran; and
when Ring B is pyrrolidine, n is 1 and X is O or S, and when Ring B is not pyrrolidine, n is 0.

[9] The compound or salt thereof according to any one of [1] to [9],
wherein Y represents an 8- to 10-membered unsaturated bicyclic ring which contains at least one heteroatom selected from the group consisting of N and S, or a 6- to 10-membered aromatic hydrocarbon ring; and
wherein the ring may be substituted by halogen atom, hydroxyl, amino, C1-C6 alkyl, C2-C3 alkenyl, C2-C3 alkynyl or 5- to 6-membered unsaturated monocyclic ring which contains at least one heteroatom selected from the group consisting of N, S and O.

[10] The compound or salt thereof according to any one of [1] to [9],
wherein Y represents benzene, naphthalene, benzo[b]thiophene, thieno[3,2-b]pyridine, isoquinoline, indole, or indazole which may be substituted by halogen atom, hydroxyl, amino, C1-C6 alkyl, C2-C3 alkenyl, C2-C3 alkynyl or thiophenyl.

[11] The compound or salt thereof according to any one of [1] to [10], wherein
L represents oxygen atom, or substituted or unsubstituted C2-C3 alkynyl;
Z represents cyanoalkyl, alkylcarbonylaminoalkyl, alkylaminocarbonyl, alkylaminoalkyl, C3-C6 cycloalkyl, a 5-to 6-membered saturated ring which contains at least one heteroatom selected from the group consisting of N, S, and O, an 8- to 10-membered partially unsaturated ring which contains at least one heteroatoms selected from the group consisting of N, S, and O;
wherein the ring in Z may be substituted by halogen atom, hydroxyl, C1-C6 alkyl, C1-C3 alkoxy, C1-C3 hydroxyalkyl, C1-C3 methoxyalkyl, a substituted or unsubstituted 5- to 6-membered saturated ring which contains at least one heteroatom selected from the group consisting of N, S, and O, and which may be substituted by C1 to C3 alkyl, alkylcarbonylalkyl, hydroxyalkyl, dialkylamino, dialkylaminoalkyl, alkoxyalkyl, or cyanoalkyl;
when L is oxygen atom, m is 0 or 1, and
when L is C2-C3 alkynyl, m is 1 and Z is dimethylaminocarbonyl or dimethylaminomethyl.

[12] The compound or salt thereof according to any one of [1] to [11], wherein
L represents oxygen atom;
m is 0 or 1;
Z represents C3-C6 cycloalkyl, a 5- to 6-membered saturated ring which contains at least one heteroatom selected from the group consisting of N, S, and O, an 8- to 10-membered partially saturated ring which contains at least one heteroatom selected from the group consisting of N, S, and 0;
wherein the ring in Z may be substituted by halogen atom, hydroxyl, C1-C6 alkyl, C1-C3 alkoxy, C2-C3 alkynyl, alkylcarbonylalkyl, hydroxyalkyl, dialkylamino, dialkylaminoalkyl, alkoxyalkyl, cyanoalkyl or C1-C6 alkyl which is substituted by 5- to 6-membered saturated ring which contains at least one heteroatom selected from the group consisting of N, S, and O and which may be further substituted by halogen atom.

[13] The compound or salt thereof according to any one of [1] to [12], wherein
L represents oxygen atom;
m is 1;
Z represents C3-C6 cycloalkyl or a 5- to 6-membered saturated ring which contains at least one heteroatom selected from the group consisting of N, S, and, 0;
wherein the ring in Z may be substituted by halogen atom, hydroxyl, cyano, C1-C6 alkyl, C1-C3 alkoxy, alkylcarbonylalkyl, hydroxyalkyl, dialkylamino, dialkylaminoalkyl, alkoxyalkyl, cyanoalkyl, or C1-C6 alkyl which is substituted by 5- to 6-membered saturated ring which contains at least one heteroatom selected from the group consisting of N, S, and O and which may be further substituted by halogen atom.

[14] The compound or salt thereof according to any one of [1] to [13], wherein
Z represents:
cyclobutane, cyclopropane, piperidine, morpholine, piperazine, isoindoline or 1,2,3,4-tetrahydroisoquinoline, and which may be substituted by halogen atom, hydroxyl, cyano, C1-C6 alkyl, or C1-C3 alkoxy; alkylcarbonylalkyl, hydroxyalkyl, dialkylamino, dialkylaminoalkyl, alkoxyalkyl, cyanoalky or C1-C6 alkyl which is substituted by 5- to 6-membered saturated ring which contains at least one heteroatom selected from the group consisting of N and O and which may be further substituted by halogen atom.

[15] The compound or salt thereof according to any one of [1] to [14], wherein
Z represents cyclobutane, cyclopropane, piperidine, morpholine, piperazine, isoindoline, or 1,2,3,4-tetrahydroisoquinoline which may be substituted by halogen atom, hydroxyl, C1-C3 alkoxy, methyl, ethyl, isopropanyl, ethylcalbonylmethyl, hydroxyethyl, dimethylamino, dimethylaminomethyl, methoxyethyl, cyanomethyl, morpholylmethyl, or 3-fluoropyrrolidinylmethyl.

[16] The compound or salt thereof according to any one of [1] to [15], wherein
the Ring A is represented by the formula (3a) or (3b):

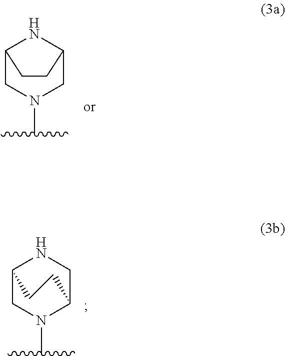 [SMILES: *N1CC2CCC(C1)N2 ; *N1C[C@@H]2CC[C@H]1CN2]

the Ring B represents benzene, piperidine, or pyrrolidine which may be substituted by halogen atom or C1-C6 alkyl;
when Ring B is pyrrolidine, n is 1 and X is 0, and when the Ring B is not pyrrolidine, n is 0;
Y represents naphthalene which may be substituted by halogen atom, hydroxyl, C1-C6 alkyl, C2-C3 alkenyl, or C2-C3 alkynyl;
L represents oxygen atom;
m is 1;
Z represents cyclobutane, cyclopropane, piperidine, morpholine, piperazine, isoindoline, or 1,2,3,4-tetrahydroisoquinoline which is substituted by halogen atom, hydroxyl, C1-C3 alkoxy, methyl, ethyl, isopropanyl, ethylcalbonylmethyl, hydroxyethyl, dimethylamino, dimethylaminomethyl, alkoxyalkyl, cyanomethyl, morpholinylmethyl, or 3-fluoropyrrolidinemethyl.

[17] The compound or salt thereof according to any one of [1] to [16], wherein the compound is selected from the following group of compounds:
(1) 4-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-5-bromonaphthalen-2-ol,
(2) 4-(4-(3,8-diazabicyclo[3.2.1]octan-3-yl)-2-((1-(((R)-3-fluoropyrrolidin-1-yl)methyl)cyclopropyl)methoxy)-5,8-dihydropyrido[3,4-d]pyrimidin-7(6H)-yl)-5-bromonaphthalen-2-ol,
(3) 4-(4-(3,8-diazabicyclo[3.2.1]octan-3-yl)-2-((1-(morpholinomethyl)cyclopropyl)methoxy)-5,8-dihydropyrido[3,4-d]pyrimidin-7(6H)-yl)-5-bromonaphthalen-2-ol,
(4) 4-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-5-methylnaphthalen-2-ol,
(5) 4-(4-(3,8-diazabicyclo[3.2.1]octan-3-yl)-2-((1-(((R)-3-fluoropyrrolidin-1-yl)methyl)cyclopropyl)methoxy)-5,8-dihydropyrido[3,4-d]pyrimidin-7(6H)-yl)-5-methylnaphthalen-2-ol,
(6) 4-(4-(3,8-diazabicyclo[3.2.1]octan-3-yl)-2-((1-(morpholinomethyl)cyclopropyl)methoxy)-5,8-dihydropyrido[3,4-d]pyrimidin-7(6H)-yl)-5-methylnaphthalen-2-ol,
(7) 4-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-5-iodonaphthalen-2-ol,
(8) 4-(4-(3,8-diazabicyclo[3.2.1]octan-3-yl)-2-((1-(((R)-3-fluoropyrrolidin-1-yl)methyl)cyclopropyl)methoxy)-5,8-dihydropyrido[3,4-d]pyrimidin-7(6H)-yl)-5-iodonaphthalen-2-ol,
(9) 4-(4-(3,8-diazabicyclo[3.2.1]octan-3-yl)-2-((1-(morpholinomethyl)cyclopropyl)methoxy)-5,8-dihydropyrido[3,4-d]pyrimidin-7(6H)-yl)-5-iodonaphthalen-2-ol,
(10) (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl) (8-ethynyl-3-hydroxynaphthalen-1-yl)methanone,
(11) (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl) (8-ethynyl-3-hydroxynaphthalen-1-yl)methanone,
(12) (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl) (3-hydroxy-8-iodonaphthalen-1-yl)methanone,
(13) (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl) (3-hydroxy-8-iodonaphthalen-1-yl)methanone,
(14) 4-(4-(3,8-diazabicyclo[3.2.1]octan-3-yl)-6-chloro-2-((1-((dimethylamino)methyl)cyclopropyl)methoxy)-8-fluoroquinazolin-7-yl)naphthalen-2-ol,
(15) 4-(4-(3,8-diazabicyclo[3.2.1]octan-3-yl)-2-((1-((dimethylamino)methyl)cyclopropyl)methoxy)-8-fluoroquinazolin-7-yl)naphthalen-2-ol,
(16) 4-(4-(3,8-diazabicyclo[3.2.1]octan-3-yl)-8-fluoro-2-(((S)-1-methylpyrrolidin-2-yl)methoxy)quinazolin-7-yl)naphthalen-2-ol,
(17) 4-(4-(3,8-diazabicyclo[3.2.1]octan-3-yl)-2-((cis-2-(dimethylamino)cyclobutyl)methoxy)-8-fluoroquinazolin-7-yl)naphthalen-2-ol,
(18) 4-(4-(3,8-diazabicyclo[3.2.1]octan-3-yl)-2-((1-((dimethylamino)methyl)cyclopropyl)methoxy)-6,8-difluoroquinazolin-7-yl)naphthalen-2-ol
(19) 4-(4-(3,8-diazabicyclo[3.2.1]octan-3-yl)-2-((1-((dimethylamino)methyl)cyclopropyl)methoxy)-6-ethyl-8-fluoroquinazolin-7-yl)naphthalen-2-ol,
(20) 4-(4-((1S,4S)-2,5-diazabicyclo[2.2.2]octan-2-yl)-2-((1-((dimethylamino)methyl)cyclopropyl)methoxy)-5,8-dihydropyrido[3,4-d]pyrimidin-7(6H)-yl)-5-bromonaphthalen-2-ol,
(21) 4-(4-(3,8-diazabicyclo[3.2.1]octan-3-yl)-2-((1-((dimethylamino)methyl)-2,2-difluorocyclopropyl)methoxy)-5,8-dihydropyrido[3,4-d]pyrimidin-7(6H)-yl)-5-bromonaphthalen-2-ol,
(22) 1-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-8-bromoisoquinolin-3-amine,
(23) 4-(4-(3,8-diazabicyclo[3.2.1]octan-3-yl)-2-((1-((dimethylamino)methyl)cyclopropyl)methoxy)-7,8-dihydro-5H-pyrano[4,3-d]pyrimidin-7-yl)-5-ethynylnaphthalen-2-ol,
(24) 1-(1-(((4-(3,8-diazabicyclo[3.2.1]octan-3-yl)-7-(8-ethynylnaphthalen-1-yl)-5,6,7,8-tetrahydroquinazolin-2-yl)oxy)methyl)cyclopropyl)-N,N-dimethylmethanamine,
(25) 4-((1-(((4-(3,8-diazabicyclo[3.2.1]octan-3-yl)-7-(8-iodonaphthalen-1-yl)-5,6,7,8-tetrahydropyrido[3,4-d]pyrimidin-2-yl)oxy)methyl)cyclopropyl)methyl)morpholine,
(26) 4-(4-(3,8-diazabicyclo[3.2.1]octan-3-yl)-2-(((R)-1-methylpyrrolidin-2-yl)methoxy)-5,8-dihydropyrido[3,4-d]pyrimidin-7(6H)-yl)-5-bromonaphthalen-2-ol,
(27) 4-(4-(3,8-diazabicyclo[3.2.1]octan-3-yl)-2-(((2S,4R)-4-fluoro-1-methylpyrrolidin-2-yl)methoxy)-5,8-dihydropyrido[3,4-d]pyrimidin-7(6H)-yl)-5-bromonaphthalen-2-ol,
(28) 4-(4-((1S,4S)-2,5-diazabicyclo[2.2.2]octan-2-yl)-2-((1-(morpholinomethyl)cyclopropyl)methoxy)-5,8-dihydropyrido[3,4-d]pyrimidin-7(6H)-yl)-5-bromonaphthalen-2-ol,
(29) 1-(1-(((4-(3,8-diazabicyclo[3.2.1]octan-3-yl)-7-(8-bromonaphthalen-1-yl)-5,6,7,8-tetrahydropyrido[3,4-d]pyrimidin-2-yl)oxy)methyl)-2,2-difluorocyclopropyl)-N,N-dimethylmethanamine,
(30) 4-(4-(3,8-diazabicyclo[3.2.1]octan-3-yl)-2-((1-((dimethylamino)methyl)-2,2-dimethylcyclopropyl)methoxy)-5,8-dihydropyrido[3,4-d]pyrimidin-7(6H)-yl)-5-bromonaphthalen-2-ol,
(31) 4-(4-(3,8-diazabicyclo[3.2.1]octan-3-yl)-2-(((S)-1-methylpyrrolidin-2-yl)methoxy)-5,8-dihydropyrido[3,4-d]pyrimidin-7(6H)-yl)-5-bromonaphthalen-2-ol,
(32) 4-(4-(3,8-diazabicyclo[3.2.1]octan-3-yl)-2-(((2S,4R)-4-methoxy-1-methylpyrrolidin-2-yl)methoxy)-5,8-dihydropyrido[3,4-d]pyrimidin-7(6H)-yl)-5-bromonaphthalen-2-ol,
(33) 4-(4-(3,8-diazabicyclo[3.2.1]octan-3-yl)-2-((1-(morpholinomethyl)cyclopropyl)methoxy)-7,8-dihydro-5H-pyrano[4,3-d]pyrimidin-7-yl)-5-ethynylnaphthalen-2-ol,
(34) (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl) (3-hydroxy-8-vinylnaphthalen-1-yl)methanone,
(35) (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl) (3-hydroxy-8-vinylnaphthalen-1-yl)methanone,
(36) 1-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-8-ethynylisoquinolin-3-amine, and
(37) 4-(4-(3,8-diazabicyclo[3.2.1]octan-3-yl)-2-((1-((dimethylamino)methyl)cyclopropyl)methoxy)-7,8-dihydro-5H-pyrano[4,3-d]pyrimidin-7-yl)-5-bromonaphthalen-2-ol.

[18] A pharmaceutical preparation comprising the compound or a salt thereof according to any one of [1] to [17].

[19] A pharmaceutical composition comprising the compound or a salt thereof according to any one of [1] to [17], and a pharmaceutically acceptable carrier.

[20] An antitumor agent comprising the compound or a salt thereof according to any one of [1] to [17] as an active ingredient.

[21] An antitumor agent for oral administration comprising the compound or a salt thereof according to any one of [1] to [17] as an active ingredient.

[22] Use of the compound or a salt thereof according to any one of [1] to [17] for the manufacture of a pharmaceutical composition.

[23] Use of the compound or a salt thereof according to any one of [1] to [17] for the manufacture of an antitumor agent.

[24] Use of the compound or a salt thereof according to any one of [1] to [17] for the manufacture of an antitumor agent for oral administration.

[25] The compound or a salt thereof according to any one of [1] to [17] for use as a pharmaceutical preparation.

[26] The compound or a salt thereof according to any one of [1] to [17] for use in a method of preventing and/or treating a tumor.

[27] The compound or a salt thereof according to any one of [1] to [17] for use in a method of preventing and/or treating a tumor by oral administration.

[28] A method for treating a tumor, the method comprising administering an effective amount of the compound or a salt thereof according to any one of [1] to [17] to a subject in need thereof.

[29] An antitumor agent comprising the compound or a salt thereof according to any one of [1] to [17], wherein the agent is administered to a subject in need thereof in combination with a therapeutically effective amount of one or more other antitumor drugs.

[30] The antitumor agent of [29], wherein the tumor is a cancer.

[31] The antitumor agent of [30], wherein the cancer is at least one selected from the group consisting of carcinoma, squamous carcinoma, adenocarcinoma, sarcoma, leukemia, neuroma, melanoma, and lymphoma.

[32] The antitumor agent of [31], wherein the squamous carcinoma is a cancer of uterine cervix, tarsus, conjunctiva, vagina, lung, oral cavity, skin, bladder, tongue, larynx or esophagus.

[33] The antitumor agent of [31], wherein the adenocarcinoma is a cancer of prostate, small intestine, endometrium, uterine cervix, large intestine, lung, pancreas, esophagus, rectum, uterus, stomach, breast or ovary.

[34] The antitumor agent of [30], wherein the cancer is lung cancer, pancreatic cancer, rectal cancer, colon cancer, colorectal cancer or uterine cancer.

[35] An antitumor agent comprising a compound or a pharmaceutically acceptable salt thereof according to any one of [1] to [17], and one or more other antitumor agents as an active ingredient.

[36] An antitumor agent comprising a compound or a pharmaceutically acceptable salt thereof according to any one of [1] to [17] as an active ingredient, which is administered in combination with one or more other antitumor agents.

### MODES FOR CARRYING OUT THE INVENTION

The present invention relates to inhibitors of KRAS G12D (referred to as “KRAS G12D inhibitor”). In particular, the present invention relates to compounds that inhibit the activity of KRAS G12D, pharmaceutical compositions comprising a therapeutically effective amount of the compounds and methods of use therefor.

A compound represented by formula (1) or a salt thereof impairs the KRAS function in KRAS G12D mutation-positive cancer cells, thereby showing antitumor action; therefore, a compound represented by formula (1) or a salt thereof can be used as an anti-cancer agent.

The compound represented by formula (1) above of the present invention is a novel compound that is nowhere disclosed in any of the literature cited above.

Unless defined otherwise, all technical and scientific terms used herein have the same meaning as is commonly understood by one of skill in the art to which this invention belongs.

All patents, patent applications, and publications referred to herein are incorporated by reference.

As used herein, unless otherwise specified, examples of the “substituent” include hydrogen atom, halogen atom, cyano, nitro, amino, hydroxyl, alkyl, hydroxyalkyl, cycloalkyl, C2-4 linear or branched hydrocarbon, alkenyl, alkynyl, alkoxy, benzyl, alkoxyalkyl, alkoxycarbonyl, alkylamino, dialkylamino, alkylaminoalkyl, carboxy, alkylcarbonyl, alkoxycarbonyl, alkylcarbonylaminoalkyl, alkylaminocarbonyl, alkylaminoalkyl, a saturated or unsaturated ring, saturated or unsaturated monocyclic or bicyclic ring, aromatic hydrocarbon, and the like. Unless otherwise specified, when a substituent listed above is present, they may be the same or different, and the number of them is typically one, two, or three.

As used herein, specific examples of the “halogen atom” include chlorine, bromine, fluorine, and iodine, with chlorine, bromine, fluorine, and iodine being preferable.

As used herein, the term “alkyl” refers to a linear or branched saturated hydrocarbon group. Examples of alkyl include C1-C6 alkyl, such as methyl, ethyl, n-propyl, isopropyl, n-butyl, isobutyl, sec-butyl, tert-butyl, n-pentyl, isopentyl, and hexyl. The “alkyl” is preferably methyl, ethyl, n-propyl, isopropyl, n-butyl, isobutyl, or tert-butyl.

As used herein, the term “hydroxyalkyl” refers to alkyl mentioned above having at least one hydroxy group (preferably having 1 to 10, and more preferably 1 to 2 hydroxy groups). Examples of hydroxyalkyl include C1-C6 hydroxyalkyl, such as hydroxymethyl, hydroxyethyl, 1-hydroxypropyl, and 2-hydroxybutyl. The “hydroxyalkyl” is preferably hydroxymethyl or hydroxyethyl.

As used herein, the term “cyanoalkyl” refers to alkyl mentioned above having at least one cyano group (preferably having 1 to 10, and more preferably 1 to 2 cyano groups). Examples of cyanoalkyl include C1-C6 cyanoalkyl, such as cyanomethyl, cyanoethyl, 1-cyanopropyl, and 2-cyanobutyl. The “cyanoalkyl” is preferably cyanomethyl or cyanoethyl.

As used herein, the term “cycloalkyl” refers to monocyclic or polycyclic saturated hydrocarbon. Examples of cycloalkyl include C3-C10 cycloalky, such as cyclopropyl, cyclobutyl, cyclopentyl, cyclohexyl, cycloheptyl, and cyclodecyl, with cyclopropyl, cyclobutyl, and cyclopentyl being preferable, and cyclopropyl, and cyclobutyl being particularly preferable.

As used herein, the term “cycloalkenyl” refers to monocyclic or polycyclic unsaturated hydrocarbon containing at least one carbon-carbon double bond (e.g., one to two carbon-carbon double bonds, and preferably one carbon-carbon double bond). Examples of cycloalkenyl include C4-C10 cycloalkenyl, such as cyclopropenyl, cyclobutenyl, cyclopentenyl, cyclohexenyl, cycloheptenyl, and cyclodecenyl, with cyclohexenyl being preferable.

As used herein, the term “unsaturated hydrocarbon” refers to linear or branched unsaturated hydrocarbon containing at least one carbon-carbon double bond or triple bond. Examples of unsaturated hydrocarbon include C2-C6 unsaturated hydrocarbon, such as vinyl, allyl, methylvinyl, 1-propenyl, butenyl, pentenyl, hexenyl, ethynyl, and 2-propynyl, with C2-4 linear or branched hydrocarbon containing at least one carbon-carbon double bond or triple bond being preferable, and vinyl, allyl, and 1-propenyl being more preferable.

As used herein, the term “alkenyl” refers to a linear or branched unsaturated hydrocarbon group containing at least one double bound (e.g., one to two double bonds, and preferably one double bond). Examples of alkenyl include C2-C6 alkenyl, such as vinyl, allyl, 1-propenyl, 2-methyl-2-propenyl, isopropenyl, 1-, 2-, or 3-butenyl, 2-, 3- or 4-pentenyl, 2-methyl-2-butenyl, 3-methyl-2-butenyl, and 5-hexenyl, with vinyl, allyl, 1-propenyl, and 2-methyl-2-propenyl being preferable.

As used herein, the term “alkynyl” refers to linear or branched unsaturated hydrocarbon containing at least one triple bond (e.g., one or two triple bonds, and preferably one triple bond). Examples of alkynyl include C2-C6 alkynyl groups, such as ethynyl, 1- or 2-propynyl, 1-, 2-, or 3-butynyl, and 1-methyl-2-propynyl, with ethynyl and 2-propynyl being preferable.

As used herein, the term “alkoxy” refers to oxy having alkyl mentioned above. Examples of alkoxy include C1-C3 alkoxy, such as methoxy, ethoxy, n-propoxy, and isopropoxy with methoxy and ethoxy being preferable, and methoxy being more preferable.

As used herein, the term “alkoxyalkyl” refers to alkyl mentioned above having at least one alkoxy group mentioned above. Examples of alkoxyalkyl include C1-C3 alkoxy-C1-C6 alkyl, such as methoxymethyl, ethoxyethyl, methoxyethyl, and methoxypropyl.

As used herein, the term “alkylamino” refers to amino having one or two alkyl groups mentioned above. Specific examples of alkylamino include C1-C6 alkylamino, such as methylamino, ethylamino, dimethylamino, diethylamino, and ethylmethylamino, with methylamino and dimethylamino being preferable.

As used herein, the term “alkylaminoalkyl” refers to alkyl mentioned above having at least one alkylamino group mentioned above. Examples of alkylaminoalkyl include C1-C6 alkylamino-C1-C6 alkyl, such as methylaminomethyl, methylaminoethyl, ethylaminomethyl, and ethylaminopropyl.

As used herein, the term “alkylaminocarbonyl” refers to calbonyl mentioned above having at least one alkylamino group mentioned above. Examples of alkylaminocarbonyl include C1-C6 alkylamino-C1-C6 alkyl, such as methylaminocarbonyl, and ethylaminocarbonyl.

As used herein, the term “dialkylamino” refers to amino having two alkyl groups mentioned above. Examples of dialkylamino include C2-C12 dialkylamino, such as dimethylamino, diethylamino, di(n-propyl)amino, diisopropylamino, di(n-butyl)amino, diisobutylamino, di(tert-butyl)amino, di(n-pentyl)amino, diisopentylamino, dihexylamino, methylethylamino, and methylisopropylamino, with dimethylamino being preferable.

As used herein, the “aromatic hydrocarbon” refers to monocyclic or polycyclic aromatic hydrocarbon as being an unsaturated bond-containing ring substituent containing carbon and hydrogen, the monocyclic or polycyclic aromatic hydrocarbon containing 4e+2 number of electrons (e is an integer of 1 or more) in the cyclic n electron system. Examples of aromatic hydrocarbon include phenyl, naphthyl, tetrahydronaphthyl, anthracenyl, and the like.

As used herein, the term “alkylcarbonyl” refers to carbonyl having alkyl mentioned above. Examples of alkylcarbonyl include C1-C6 alkylcarbonyl, such as methylcarbonyl, ethylcarbonyl, n-propylcarbonyl, isopropylcarbonyl, n-butylcarbonyl, isobutylcarbonyl, tert-butylcarbonyl, n-pentylcarbonyl, isopentylcarbonyl, and hexylcarbonyl, with methylcarbonyl being preferable.

As used herein, the term “alkylcarbonylalkyl” refers to alkyl having alkylcarbonyl mentioned above. Examples of alkylcarbonylalkyl include C1-C6 alkylcarbonyl, such as methylcarbonylmethyl, ethylcarbonylmethyl, n-propylcarbonylmethyl, isopropylcarbonylmethyl, n-butylcarbonylmethyl, isobutylcarbonylmethyl, tert-butylcarbonylmethyl, n-pentylcarbonylmethyl, isopentylcarbonylmethyl, and hexylcarbonylmethyl, with methylcarbonylmethyl and ethylcarbonylmethyl being preferable.

As used herein, the term “alkylcarbonylaminoalkyl” refers to aminoalkyl having alkylcarbonyl mentioned above. Examples of alkylcarbonylaminoalkyl include C1-C6 alkylcarbonylaminoalkyl, such as methylcarbonylaminomehtyl and ethylcarbonylaminomehtyl, with methylcarbonylmethyl being preferable.

As used herein, the term “alkoxycarbonyl” refers to carbonyl having alkoxy mentioned above. Examples of alkoxycarbonyl include C1-C6 alkoxycarbonyl, such as methoxycarbonyl, ethoxycarbonyl, propoxycarbonyl, isopropoxycarbonyl, butoxycarbonyl, isobutoxycarbonyl, tert-butoxycarbonyl, pentyloxycarbonyl, isopentyloxycarbonyl, and hexyloxycarbonyl, with methoxycarbonyl being preferable.

As used herein, the term “saturated ring” as substituent refers to a monocyclic or polycyclic saturated ring containing at least one heteroatom (preferably having 1 to 5, and more preferably 1 to 3 heteroatoms) selected from nitrogen, oxygen, and sulfur. Examples of saturated ring include aziridinyl, azetidinyl, imidazolidinyl, morpholino, pyrrolidinyl, piperidinyl, piperazinyl, tetrahydrofuranyl, tetrahydro-2H-pyranyl, oxetanyl, tetrahydropyranyl, tetrahydrothiophenyl, thiazolidinyl, oxazolidinyl, and the like, with pyrrolidinyl, piperidinyl, piperazinyl, morpholino, tetrahydrofuranyl, tetrahydro-2H-pyranyl, and oxetanyl being preferable.

As used herein, the term “unsaturated ring” as substituent refers to a monocyclic or polycyclic, completely or partially unsaturated ring group containing at least one heteroatom (preferably containing 1 to 5, and more preferably 1 to 3 heteroatoms) selected from nitrogen, oxygen, and sulfur. Examples of unsaturated ring include imidazolyl, thienyl, pyrrolyl, oxazolyl, isoxazolyl, thiazolyl, isothiazolyl, thiadiazolyl, oxadiazolyl, pyrazolyl, triazolyl, tetrazolyl, pyridyl, pyrazyl, pyrimidinyl, pyridazinyl, indolyl, isoindolyl, indazolyl, triazolopyridyl, benzoimidazolyl, benzoxazolyl, benzothiazolyl, benzothienyl, furanyl, benzofuranyl, purinyl, quinolyl, isoquinolyl, quinazolinyl, quinoxalyl, methylenedioxyphenyl, ethylenedioxyphenyl, dihydrobenzofuranyl, 1,2,3,4-tetrahydroisoquinolyl and the like, with imidazolyl, thienyl pyrazolyl, thiazolyl, isoxazolyl, furanyl, isoindolyline and 1,2,3,4-tetrahydroisoquinolyl being preferable, and thienyl, isoindolylyl and 1,2,3,4-tetrahydroisoquinolyl being more preferable.

As used herein, the term “CA-CB” indicates that the number of carbon atoms of A to B in a certain group. For example, “C1-C6 alkyl” refers to alkyl having 1 to 6 carbon atoms, and “C6-C14 aromatic hydrocarbon oxy” refers to oxy to which C6-C14 aromatic hydrocarbon is bonded. Further, the term “A-to B-membered” indicates that the number of atoms (number of ring members) that constitute a ring is A to B. More specifically, “4- to 10-membered saturated ring heterocyclic group” refers to a saturated ring containing 4 to 10 ring members.

In one aspect of the invention, compounds are provided represented by formula (1):

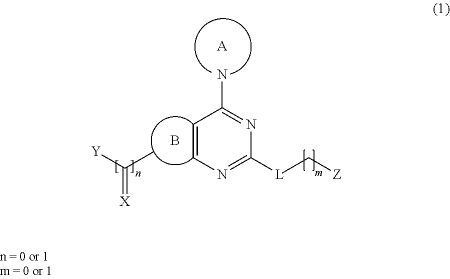

or a pharmaceutically acceptable salt thereof, wherein:
Ring A represents a substituted or unsubstituted, saturated or unsaturated 8- to 10-membered N-containing bridged ring which contains at least one further heteroatom selected from the group consisting of N, S and O;
Ring B represents a substituted or unsubstituted, 5- to 6-membered saturated or unsaturated ring having at least one heteroatom selected from the group consisting of N, S, and O, a 6-membered aromatic hydrocarbon ring, C3-C6 cycloalkyl ring, C3-C6 cycloalkenyl or an 8- to 10-membered spiro ring, wherein the Ring B is fused with the pyrimidine ring to form a substituted or unsubstituted bicyclic ring;
n is 0 or 1;
X is O or S;
Y represents a substituted or unsubstituted, 6- to 10-membered unsaturated monocyclic or bicyclic ring which contains at least one heteroatom selected from the group consisting of N, S and O, or 6- to 10-membered aromatic hydrocarbon ring;
L represents oxygen atom, or a substituted or unsubstituted C2-C3 alkynyl;
Z represents cyanoalkyl, alkylcarbonylaminoalkyl, alkylaminocarbonyl, alkylaminoalkyl, a substituted or unsubstituted, C3-C6 cycloalkyl, a 5- to 6-membered saturated ring which contains at least one heteroatom selected from the group consisting of N, S and O, or an 8-to 10-membered partially unsaturated ring which contains at least one heteroatom selected from the group consisting of N, S and O;
when L is C2-C3 alkynyl, Z is alkylaminocarbonyl or alkylaminoalkyl;
m is 0 or 1.

Ring A

In the compound represented by formula (1) of the present invention, Ring A represents a substituted or unsubstituted, saturated or unsaturated 8- to 10-membered N-containing bridged ring which contains at least one further heteroatom selected from the group consisting of N, S and O.

The “saturated 8- to 10-membered N-containing bridged ring” is preferably a saturated monocyclic 8- to 10-membered N-containing bridged ring further containing 1 to 5 heteroatoms selected from N, S, and O, more preferably a saturated monocyclic 8-membered N-containing bridged ring further containing at least one heteroatom selected from N, S, and O, and more preferably piperazinyl ring-based, 8-membered N-containing bridged ring, and more preferably diazabicyclo[3.2.1]octane and diazabicyclo[2.2.2]octane, and more preferably diazabicyclo[3.2.1]octane.

The “unsaturated 8- to 10-membered N-containing bridged ring” is preferably a unsaturated monocyclic 8- to 10-membered N-containing bridged ring further containing 1 to 5 heteroatoms selected from N, S, and O, more preferably a unsaturated monocyclic 8-membered N-containing bridged ring further containing at least one heteroatom selected from N, S, and O, and more preferably piperazinyl ring-based, 8-membered N-containing bridged ring, and more preferably diazabicyclo[3.2.1]oct-6-ene.

The substituent in the “substituted 8- to 10-membered N-containing bridged ring” may be, for example, the substituents mentioned above, and is preferably hydrogen atom, C1-C6 alkyl, hydroxyl, halogen atom, alkoxycarbonyl, cyano, nitro or hydroxyalkyl.

The substituent in the “substituted 8- to 10-membered N-containing bridged ring” is also represented by R₁ or R₂ in formulae (2a) to (2c) of the present invention. R₁ may be hydrogen atom or hydroxyl, and preferably hydrogen atom or hydroxyl, and more preferably hydrogen atom. R₂ may be hydrogen atom, halogen atom, alkoxycarbonyl, cyano, nitro, or hydroxyalkyl, and preferably hydrogen atom, alkoxycarbonyl, cyano, nitro, or hydroxyalkyl, and more preferably hydrogen atom.

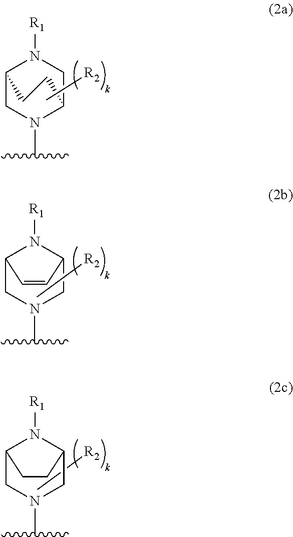

The “alkoxycarbonyl” included in the substituent of Ring A is preferably methoxycarbonyl or ethoxycarbonyl, more preferably methoxycarbonyl.

The “hydroxyalkyl” included in the substituents of Ring A is preferably hydroxymethyl or hydroxyethyl, more preferably hydroxymethyl.

The “halogen atom” included in substituents of Ring A is preferably fluorine, chlorine, bromine or iodide.

In Ring A represented by formulae (2a) to (2c) of the present invention, k is 0 to 6, and preferably 0 to 5, more preferably 0 to 4, more preferably 0 to 3, more preferably 0 to 2, more preferably 0 or 1, particularly preferably 0.

Ring B

In the compound represented by formula (1) of the present invention, Ring B represents a substituted or unsubstituted, 5- to 6-membered saturated or unsaturated ring having at least one heteroatom selected from the group consisting of N, S, and O, a 6-membered aromatic hydrocarbon ring, C3-C6 cycloalkyl ring, C3-C6 cycloalkenyl or an 8- to 10-membered spiro ring, wherein the Ring B is fused with the pyrimidine ring to form a substituted or unsubstituted bicyclic ring.

The “5- to 6-membered saturated ring having at least one heteroatom selected from the group consisting of N, S, and O” is preferably a monocyclic 5- to 6-membered saturated ring having 1 to 3 heteroatoms selected from N, S, and O, more preferably a monocyclic 5- to 6-membered saturated ring having one heteroatom selected from N, S, and O, more preferably a monocyclic 5- to 6-membered saturated ring having one heteroatom selected from N and 0, and more preferably piperidine, pyrrolidine, or tetrahydro-2H-pyran, and particularly preferably piperidine or pyrrolidine.

The “5- to 6-membered unsaturated ring having at least one heteroatom selected from the group consisting of N, S, and O” is preferably a monocyclic 5- to 6-membered unsaturated ring having 1 to 3 heteroatoms selected from N, S, and O, more preferably a monocyclic 5- to 6-membered unsaturated ring having one heteroatom selected from N, S, and O, more preferably a monocyclic 5- to 6-membered unsaturated ring having one heteroatom selected from N and 0, and more preferably 2,3-dihydrofuran, 3,4-dihydro-2H-pyran or 4H-pyran, and particularly preferably 3,4-dihydro-2H-pyran.

The “4- to 6-membered saturated monocyclic ring which contains at least one heteroatom selected from N, S, and O.” is preferably a monocyclic 4- to 6-membered saturated ring having 1 to 3 heteroatoms selected from N, S, and O, more preferably a monocyclic 4- to 6-membered saturated ring having one heteroatom selected from N, S, and O, more preferably a monocyclic 4- to 6-membered saturated ring having one heteroatom selected from N and 0, and more preferably oxetanyl, tetrahytdrofuranyl or tetrahydro-2H-pyranyl.

The substituent in the “substituted 5- to 6-membered saturated or unsaturated ring having at least one heteroatom selected from the group consisting of N, S, and O” may be, for example, the substituents mentioned above, and is preferably, halogen atom, C1-C6 alkyl, alkylcarbonyl or 4-to 6-membered saturated monocyclic ring which contains at least one heteroatom selected from N, S, and O.

The “halogen atom” included in substituents of Ring B is preferably fluorine, chlorine, bromine or iodide.

The “C1-C6 alkyl” included in substituents of Ring B is preferably methyl, ethyl, n-propyl, or isopropyl (C1-C3 alkyl), more preferably methyl or ethyl.

The “alkylcarbonyl” included in substituents of Ring B is preferably methoxycarbonyl or ethoxycarbonyl, more preferably methoxycarbonyl.

The “4- to 6-membered saturated monocyclic ring” in the substituents of Ring B is preferably oxetanyl, tetrahytdrofuranyl or tetrahydro-2H-pyranyl.

The “6-membered aromatic hydrocarbon ring” in the “substituted or unsubstituted 6-membered aromatic hydrocarbon ring” is preferably benzene.

The substituent in the “substituted 6-membered aromatic hydrocarbon ring” may be, for example, the substituents mentioned above, and is preferably, halogen atom, C1-C6 alkyl, alkylcarbonyl or 4- to 6-membered saturated monocyclic ring which contains at least one heteroatom selected from N, S, and O, and more preferably fluorine, chlorine, methyl, or ethyl.

The “C3-C6 cycloalkyl” in the “substituted or unsubstituted C3-C6 cycloalkyl” is preferably cyclobutyl, cyclopentyl, or cyclohexyl, and more preferably cyclohexyl.

The substituent in the “substituted C3-C6 cycloalkyl” may be, for example, the substituents mentioned above, and is preferably, a halogen atom, C1-C6 alkyl, alkylcarbonyl or 4-to 6-membered saturated monocyclic ring which contains at least one heteroatom selected from N, S, and O.

The “C3-C6 cycloalkenyl” in the “substituted or unsubstituted C3-C6 cycloalkenyl” is preferably cyclopentenyl or cyclohexenyl, and more preferably cyclohexenyl.

The substituent in the “substituted C3-C6 cycloalkenyl” may be, for example, the substituents mentioned above, and preferably, halogen atom, C1-C6 alkyl, alkylcarbonyl or 4-to 6-membered saturated monocyclic ring which contains at least one heteroatom selected from N, S, and O, and more preferably halogen atom or C1-C6 alkyl.

The “substituted or unsubstituted C3-C6 cycloalkenyl” is preferably cyclopentenyl, cyclohexenyl or cycloheptenyl, and more preferably cyclohexenyl.

The “8- to 10-membered spiro ring” in the “substituted or unsubstituted 8- to 10-membered spiro ring” is preferably spiro[2.5]octane, spiro[3.5]nonane or spiro[4.5]decane, and more preferably spiro[2.5]octane.

The substituent in the “substituted or 8- to 10-membered spiro ring” may be, for example, the substituents mentioned above, and preferably halogen atom, C1-C6 alkyl, alkylcarbonyl or 4- to 6-membered saturated monocyclic ring which contains at least one heteroatom selected from N, S, and O.

In the compound represented by formula (1) of the present invention, when Ring B is pyrrolidine, n is 1 and X is O or S, and when Ring B is not pyrrolidine, n is 0.

In the compound represented by formula (1) of the present invention, Ring B is fused with the pyrimidine ring to form a substituted or substituted bicyclic ring. Examples of the bicyclic ring include, but not limited to, quinazoline, 5,6,7,8-tetrahydropyrido[3,4-d]pyrimidine, 6,7-dihydro-5H-pyrrolo[3,4-d]pyrimidine, 5,6,7,8-tetrahydroquinazoline, 7′,8′-dihydro-5′H-spiro[cyclopropane-1,6′-quinazoline], 7,8-dihydroquinazoline, 5,6-dihydroquinazoline, 7,8-dihydro-5H-pyrano[4,3-d]pyrimidine, 5H-pyrano[4,3-d]pyrimidine, 5,6,7,8-tetrahydropyrido[2,3-d]pyrimidine, 6,7-dihydro-5H-pyrano[2,3-d]pyrimidine, and 5,6,7,8-tetrahydropyrido[4,3-d]pyrimidine, and more preferably, quinazoline, 5,6,7,8-tetrahydropyrido[3,4-d]pyrimidine, 6,7-dihydro-5H-pyrrolo[3,4-d]pyrimidine, 7,8-dihydro-5H-pyrano[4,3-d]pyrimidine and 5H-pyrano[4,3-d]pyrimidine.

The substituent in the “fused with the pyrimidine ring to form a substituted or substituted bicyclic ring” may be, for example, the substituents mentioned above, and preferably halogen atom, Ca—C6 alkyl, C1-C3 alkenyl, alkylcarbonyl, or 4- to 6-membered saturated monocyclic ring which contains at least one heteroatom selected from N, S, and O, and more preferably fluorine, chlorine, methyl, ethyl, methylcarbonyl, oxetanyl.

In the compound represented by formula (1) of the present invention, X represents O or S, and preferably O.

Monocyclic or Bicyclic Ring Defined by “Y”

In the compound represented by formula (1) of the present invention, Y represents a substituted or unsubstituted, 6-to 10-membered unsaturated monocyclic or bicyclic ring which contains at least one heteroatom selected from the group consisting of N, S and O, or 6- to 10-membered aromatic hydrocarbon ring.

The “6- to 10-membered unsaturated bicyclic ring” in the “substituted or unsubstituted, 6- to 10-membered unsaturated bicyclic ring” is preferably a bicyclic 6- to 10-membered unsaturated ring containing 1 to 5 heteroatoms selected from N, S and O, more preferably a bicyclic 6- to 10-membered unsaturated ring containing 1 to 3 heteroatoms selected from N and S, and more preferably, benzo[b]thiphene, isoquinoline, thieno[2,3-c]pyridine, indole, or indazole.

The substituent in the “substituted 6- to 10-membered unsaturated bicyclic ring” may be, for example, the substituents mentioned above, halogen atom, hydroxyl, amino, C1-C6 alkyl, C2-C3 alkenyl, C2-C3 alkynyl or 5- to 6-membered unsaturated monocyclic ring which contains at least one heteroatom selected from the group consisting of N, S and O, more preferably halogen atom, hydroxyl, amino, C1-C6 alkyl, C2-C3 alkenyl, C2-C3 alkynyl or thiophenyl, and more preferably bromine, fluorine, chlorine, iodine, hydroxyl, amino, methyl, vinyl, ethynyl or thiophenyl.

The “6- to 10-membered aromatic hydrocarbon ring” is preferably benzene or naphthalene, and more preferably naphthalene.

The substituent in the “substituted 6-membered aromatic hydrocarbon ring” may be, for example, the substituents mentioned above, halogen atom, hydroxyl, amino, C1-C6 alkyl, C2-C3 alkenyl, C2-C3 alkynyl or 5- to 6-membered unsaturated monocyclic ring which contains at least one heteroatom selected from the group consisting of N, S and O, and more preferably halogen atom, hydroxyl, amino, C1-C6 alkyl, C2-C3 alkenyl, C2-C3 alkynyl or thiophenyl.

The “halogen atom” included in the substituents of Y is preferably fluorine, chlorine, bromine, or iodide.

The “C1-C6 alkyl” included in the substituents of Y is preferably methyl, ethyl, n-propyl, or isopropyl (C1-C3 alkyl), more preferably methyl or ethyl.

The “C2-C3 alkenyl” included in the substituents of Y is preferably vinyl, 1-propenyl, allyl, and more preferably vinyl.

The “C2-C3 alkynyl” included in the substituents of Y is preferably ethynyl or 1-propynyl, and more preferably ethynyl.

The “5- to 6-membered unsaturated monocyclic ring” is preferably 5- to 6-membered unsaturated monocyclic ring which contains at least one heteroatom selected from the group consisting of N, S and O, and more preferably thiophenyl.

“L”

In the compound represented by formula (1) of the present invention, L represents an oxygen atom, or a substituted or unsubstituted C2-C3 alkynyl.

The “C2-C3 alkynyl” is preferably ethynyl or 1-propynyl, and more preferably ethynyl.

In the compound represented by formula (1) of the present invention, when L represents an oxygen atom, m is 0 or 1, and preferably m is 1.

The substituent in the “substituted C2-C3 alkenyl” represented by L may be, for example, the substituents mentioned above, and preferably dimethylaminomethyl or dimethylaminocarbonylmethyl.

“Z”

In the compound represented by formula (1) of the present invention, Z represents cyanoalkyl, alkylcarbonylaminoalkyl, alkylaminocarbonyl, alkylaminoalkyl, a substituted or unsubstituted, C3-C6 cycloalkyl, a 5- to 6-membered saturated ring which contains at least one heteroatom selected from the group consisting of N, S and O, or an 8-to 10-membered partially unsaturated ring which contains at least one heteroatom selected from the group consisting of N, S and O.

The “cyanoalkyl” is preferably cyanomethyl or cyanoethyl, and more preferably cyanomethyl.

The “alkylcarbonylaminoalkyl” is preferably methylcarbonylaminomehtyl, ethylcarbonylaminomehtyl, or ethylcarbonylaminoehtyl, and more preferably methylcarbonylaminomehtyl.

The “alkylaminocarbonyl” is preferably dimethylaminocarbonyl, methylaminocarbonyl or diethylaminocarbonyl, and more preferably dimethylaminocarbonyl.

The “alkylaminoalkyl” in the “substituted or unsubstituted alkylaminoalkyl” is preferably dimethylaminomethyl, dimethylaminoethyl, methylaminoethyl, or diethylaminoethyl, and more preferably dimethylaminomethyl or dimethylaminoethyl.

The “C3-C6 cycloalkyl” in the “substituted or unsubstituted C3-C6 cycloalkyl” is preferably cyclopropyl, cyclobutyl, cyclopentyl, and more preferably cyclopropyl or cyclobutyl, and more preferably cyclopropyl.

The substituent in the “substituted C3-C6 cycloalkyl” may be, for example, the substituents mentioned above, and is preferably halogen atom, hydroxyl, C1-C6 alkyl, C1-C3 alkoxy, a substituted or unsubstituted 5- to 6-membered saturated ring which contains at least one heteroatom selected from the group consisting of N, S, and O, and which may be substituted by C1 to C3 alkyl, alkylcarbonylalkyl, hydroxyalkyl, dialkylamino, dialkylaminoalkyl, or cyanoalkyl, and more preferably halogen atom, hydroxyl, methoxy, methyl, ethyl, isopropanyl, ethylcalbonylmethyl, hydroxyethyl, dimethylamino, dimethylaminomethyl, cyanomethyl, morpholylmethyl, or 3-fluoropyrrolidinylmethyl.

The “5- to 6-membered saturated ring” in the “substituted or unsubstituted 5- to 6-membered saturated ring” is preferably a 5- to 6-membered saturated ring which contains at least one heteroatom selected from the group consisting of N, S and O, and more preferably a 5- to 6-membered saturated ring containing 1 to 3 heteroatoms selected from N, and 0, and more preferably tetrahydrofuranyl, tetrahydro-2H-pyranyl, pyrrolidinyl, piperidinyl, morpholiyl, or piperazinyl.

The substituent in the “substituted 5- to 6-membered saturated ring” may be, for example, the substituents mentioned above, and is preferably halogen atom, hydroxyl, C1-C6 alkyl, C1-C3 alkoxy, alkylcarbonylalkyl, hydroxyalkyl, dialkylamino, dialkylaminoalkyl, alkoxyalkyl, or cyanoalkyl, and more preferably halogen atom, hydroxyl, methoxy, methyl, ethyl, isopropanyl, ethylcalbonylmethyl, hydroxyethyl, dimethylamino, dimethylaminomethyl, or metoxyethyl, cyanomethyl.

The substituent in the “C1-C6 alkyl which is substituted by 5- to 6-membered saturated ring which contains at least one heteroatom selected from the group consisting of N, S, and, O and which may be further substituted by halogen atom” is preferably morpholylmethyl, or 3-fluoropyrrolidinylmethyl.

The “8- to 10-membered partially unsaturated ring” in the “substituted or unsubstituted 8- to 10-membered partially unsaturated ring” is preferably 8- to 10-membered partially unsaturated ring which contains at least one heteroatom selected from the group consisting of N, S and O, and more preferably 8- to 10-membered partially unsaturated ring which contains one heteroatom selected from the group consisting of N, S and O, and more preferably isoindoline or 1,2,3,4-tetrahydroisoquinoline.

The substituent in the “substituted 8- to 10-membered partially unsaturated ring” is preferably C1-C6 alkyl, and more preferably methyl or ethyl, and more preferably methyl.

The “substituted 8- to 10-membered partially unsaturated ring” is preferably substituted or unsubstituted 8- to 10-membered partially unsaturated ring which contains at least one heteroatom selected from the group consisting of N, S and O, and more preferably 2-methylisoindoline or 2-methyl-1,2,3,4-tetrahydroisoquinoline.

The “halogen atom” included in the substituents of Z is preferably fluorine, chlorine, bromine, or iodide.

The “C1-C6 alkyl” included in the substituents of Z is preferably methyl, ethyl, propyl or isopropanyl, and more preferably methyl, ethyl or isopropanyl.

The “C1-C3 alkoxy” included in the substituents of Z is preferably methoxy or ethoxy, and more preferably methoxy.

The “alkylcarbonylalkyl” included in the substituents of Z is preferably methylcarbonylmethyl, ethylcarbonylmethyl or ethylcarbonylethyl, and more preferably ethylcarbonylmethyl.

The “hydoroxyalkyl” included in the substituents of Z is preferably hydroxymethyl, hydroxyethyl or hydroxylpropyl, and more preferably hydroxymethyl.

The “alkoxyalkyl” included in the substituents of Z is preferably methoxyethyl, methoxymethyl or ethoxyethyl, and more preferably methoxyethyl.

The “cyanoalkyl” included in the substituents of Z is preferably cyanomethyl or cyanothyl, and more preferably cyanomethyl.

The “alkylcarbonylaminoalkyl” included in the substituents of Z is preferably methylcarbonylaminomehtyl, ethylcarbonylaminomehtyl, or ethylcarbonylaminoehtyl, and more preferably methylcarbonylaminomehtyl.

The “alkylaminocarbonyl” included in the substituents of Z is preferably dimethylaminocarbonyl, methylaminocarbonyl or diethylaminocarbonyl, and more preferably dimethylaminocarbonyl.

The “alkylaminoalkyl” included in the substituents of Z is preferably dimethylaminoethyl, methylaminoethyl, or diethylaminoethyl, and more preferably dimethylaminoethyl.

The compound or a salt thereof of the present invention is preferably a compound represented by formula (1) or a salt thereof,
wherein the Ring A is represented by any one of the formulae (2a) to (2c) which may be substituted by R1 and R2:

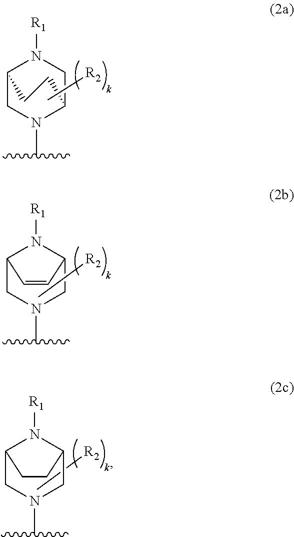

wherein R1 represents hydrogen atom, C1-C6 alkyl, or hydroxyl; R2 represents hydrogen atom, alkoxycarbonyl, cyano, or hydroxyalkyl; and
k is 0 to 1;
Ring B represents a substituted or unsubstituted, 5- to 6-membered saturated or unsaturated ring having at least one heteroatom selected from the group consisting of N, S, and O, a 6-membered aromatic hydrocarbon ring, C3-C6 cycloalkyl ring, C3-C6 cycloalkenyl or an 8- to 10-membered spiro ring, wherein the Ring B is fused with the pyrimidine ring to form a substituted or unsubstituted bicyclic ring;
n is 0 or 1;
X is O or S;
Y represents a substituted or unsubstituted, 6- to 10-membered unsaturated monocyclic or bicyclic ring which contains at least one heteroatom selected from the group consisting of N, S and O, or 6- to 10-membered aromatic hydrocarbon ring;
L represents oxygen atom, or a substituted or unsubstituted C2-C3 alkynyl;
Z represents cyanoalkyl, alkylcarbonylaminoalkyl, alkylaminocarbonyl, alkylaminoalkyl, a substituted or unsubstituted, C3-C6 cycloalkyl, a 5 to 6 membered saturated ring which contains at least one heteroatom selected from the group consisting of N, S and O, or an 8- to 10-membered partially unsaturated ring which contains at least one heteroatom selected from the group consisting of N, S and O; when L is C2-C3 alkynyl, Z is alkylaminocarbonyl or alkylaminoalkyl; and
m is 0 or 1.

The compound or a salt thereof of the present invention is preferably a compound represented by formula (1) or a salt thereof,
wherein:
Ring A is represented by the formula (3a) or (3b):

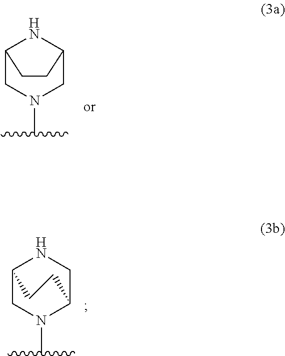 [SMILES: *N1CC2CCC(C1)N2 ; *N1C[C@@H]2CC[C@H]1CN2]

Ring B represents a substituted or unsubstituted, 5- to 6-membered saturated or unsaturated ring having at least one heteroatom selected from the group consisting of N, S, and O, a 6-membered aromatic hydrocarbon ring, C3-C6 cycloalkyl ring, C3-C6 cycloalkenyl or an 8- to 10-membered spiro ring, wherein the Ring B is fused with the pyrimidine ring to form a substituted or unsubstituted bicyclic ring;
n is 0 or 1;
X is O or S;
Y represents a substituted or unsubstituted, 6- to 10-membered unsaturated monocyclic or bicyclic ring which contains at least one heteroatom selected from the group consisting of N, S and O, or 6- to 10-membered aromatic hydrocarbon ring;
L represents oxygen atom, or a substituted or unsubstituted C2-C3 alkynyl;
Z represents cyanoalkyl, alkylcarbonylaminoalkyl, alkylaminocarbonyl, alkylaminoalkyl, a substituted or unsubstituted, C3-C6 cycloalkyl, a 5 to 6 membered saturated ring which contains at least one heteroatom selected from the group consisting of N, S and O, or an 8- to 10-membered partially unsaturated ring which contains at least one heteroatom selected from the group consisting of N, S and O; when L is C2-C3 alkynyl, Z is alkylaminocarbonyl or alkylaminoalkyl; and
m is 1.

The compound or a salt thereof of the present invention is preferably a compound represented by formula (1) or a salt thereof,
wherein:
Ring A represents a substituted or unsubstituted, saturated or unsaturated 8- to 10-membered N-containing bridged ring which contains at least one further heteroatom selected from the group consisting of N, S and O;
Ring B represents
   (i) a 5- to 6-membered saturated or unsaturated ring which contains at least one heteroatom selected from the group consisting of N, S, and O, or
   (ii) a 6- to 10-membered aromatic hydrocarbon ring,
wherein the Ring B is fused with the pyrimidine ring to form a substituted or unsubstituted bicyclic ring; and wherein the Ring B in the bicyclic ring may be substituted by halogen atom, C1-C6 alkyl, alkylcarbonyl or 4- to 6-membered saturated monocyclic ring which contains at least one hetero atom selected from N, S, and O;
n is 0 or 1;
X is O or S;
Y represents a substituted or unsubstituted, 6- to 10-membered unsaturated monocyclic or bicyclic ring which contains at least one heteroatom selected from the group consisting of N, S and O, or 6- to 10-membered aromatic hydrocarbon ring;
L represents oxygen atom, or a substituted or unsubstituted C2-C3 alkynyl;
Z represents cyanoalkyl, alkylcarbonylaminoalkyl, alkylaminocarbonyl, alkylaminoalkyl, a substituted or unsubstituted, C3-C6 cycloalkyl, a 5 to 6 membered saturated ring which contains at least one heteroatom selected from the group consisting of N, S and O, or an 8- to 10-membered partially unsaturated ring which contains at least one heteroatom selected from the group consisting of N, S and O; when L is C2-C3 alkynyl, Z is alkylaminocarbonyl or alkylaminoalkyl; and
m is 0 or 1.

The compound or a salt thereof of the present invention is preferably a compound represented by formula (1) or a salt thereof,
wherein:
Ring A represents a substituted or unsubstituted, saturated or unsaturated 8- to 10-membered N-containing bridged ring which contains at least one further heteroatom selected from the group consisting of N, S and O;
Ring B represents benzene, piperidine, pyrrolidine, tetrahydro-2H-pyran, 3,4-dihydro-2H-pyran, wherein the Ring B is fused with the pyrimidine ring to form a substituted or unsubstituted bicyclic ring;
wherein the Ring B in the bicyclic ring may be substituted by halogen atom, C1-C6 alkyl, alkylcarbonyl or 4- to 6-membered saturated monocyclic ring which contains at least one hetero atom selected from N, S, and O; and
when the Ring B is pyrrolidine, n is 1 and X is O or S, and
when the Ring B is not pyrrolidine, n is 0;
Y represents a substituted or unsubstituted, 6- to 10-membered unsaturated monocyclic or bicyclic ring which contains at least one heteroatom selected from the group consisting of N, S and O, or 6- to 10-membered aromatic hydrocarbon ring;
L represents oxygen atom, or a substituted or unsubstituted C2-C3 alkynyl;
Z represents cyanoalkyl, alkylcarbonylaminoalkyl, alkylaminocarbonyl, alkylaminoalkyl, a substituted or unsubstituted, C3-C6 cycloalkyl, a 5- to 6-membered saturated ring which contains at least one heteroatom selected from the group consisting of N, S and O, or an 8-to 10-membered partially unsaturated ring which contains at least one heteroatom selected from the group consisting of N, S and O;
when L is C2-C3 alkynyl, Z is alkylaminocarbonyl or alkylaminoalkyl; and
m is 1.

The compound or a salt thereof of the present invention is preferably a compound represented by formula (1) or a salt thereof,
wherein:
Ring A represents a substituted or unsubstituted, saturated or unsaturated 8- to 10-membered N-containing bridged ring which contains at least one further heteroatom selected from the group consisting of N, S and O;
Ring B represents
   (i) a 5- to 6-membered saturated or unsaturated ring which contains at least one heteroatom selected from the group consisting of N, S, and O,
wherein the Ring B is fused with the pyrimidine ring to form a substituted or unsubstituted bicyclic ring, and
wherein the Ring B in the bicyclic ring may be substituted by halogen atom, C1-C6 alkyl, alkylcarbonyl or 4- to 6-membered saturated monocyclic ring which contains at least one hetero atom selected from N, S, and O;
n is 0 or 1;
X is O or S;
Y represents a substituted or unsubstituted, 6- to 10-membered unsaturated monocyclic or bicyclic ring which contains at least one heteroatom selected from the group consisting of N, S and O, or 6- to 10-membered aromatic hydrocarbon ring;
L represents oxygen atom, or a substituted or unsubstituted C2-C3 alkynyl;
Z represents cyanoalkyl, alkylcarbonylaminoalkyl, alkylaminocarbonyl, alkylaminoalkyl, a substituted or unsubstituted, C3-C6 cycloalkyl, a 5- to 6-membered saturated ring which contains at least one heteroatom selected from the group consisting of N, S and O, or an 8-to 10-membered partially unsaturated ring which contains at least one heteroatom selected from the group consisting of N, S and O;
when L is C2-C3 alkynyl, Z is alkylaminocarbonyl or alkylaminoalkyl; and
m is 0 or 1.

The compound or a salt thereof of the present invention is preferably a compound represented by formula (1) or a salt thereof,
wherein:
Ring A represents a substituted or unsubstituted, saturated or unsaturated 8- to 10-membered N-containing bridged ring which contains at least one further heteroatom selected from the group consisting of N, S and O;
Ring B represents
piperidine, pyrrolidine,
wherein the Ring B is fused with the pyrimidine ring to form a substituted or unsubstituted bicyclic ring, and
wherein the Ring B in the bicyclic ring may be substituted by halogen atom, C1-C6 alkyl, alkylcarbonyl or 4- to 6-membered saturated monocyclic ring which contains at least one heteroatom selected from N, S, and O, and
when the Ring B is pyrrolidine, n is 1 and X is O or S, and
when the Ring B is not pyrrolidine, n is 0;
Y represents a substituted or unsubstituted, 6- to 10-membered unsaturated monocyclic or bicyclic ring which contains at least one heteroatom selected from the group consisting of N, S and O, or 6- to 10-membered aromatic hydrocarbon ring;
L represents oxygen atom, or a substituted or unsubstituted C2-C3 alkynyl;
Z represents cyanoalkyl, alkylcarbonylaminoalkyl, alkylaminocarbonyl, alkylaminoalkyl, a substituted or unsubstituted, C3-C6 cycloalkyl, a 5- to 6-membered saturated ring which contains at least one heteroatom selected from the group consisting of N, S and O, or an 8-to 10-membered partially unsaturated ring which contains at least one heteroatom selected from the group consisting of N, S and O;
when L is C2-C3 alkynyl, Z is alkylaminocarbonyl or alkylaminoalkyl; and
m is 1.

The compound or a salt thereof of the present invention is preferably a compound represented by formula (1) or a salt thereof,
wherein:
Ring A represents a substituted or unsubstituted, saturated or unsaturated 8- to 10-membered N-containing bridged ring which contains at least one further heteroatom selected from the group consisting of N, S and O;
Ring B represents a substituted or unsubstituted, 5- to 6-membered saturated or unsaturated ring having at least one heteroatom selected from the group consisting of N, S, and O, a 6-membered aromatic hydrocarbon ring, C3-C6 cycloalkyl ring, C3-C6 cycloalkenyl or an 8- to 10-membered spiro ring, wherein the Ring B is fused with the pyrimidine ring to form a substituted or unsubstituted bicyclic ring;
n is 0 or 1;
X is O or S;
Y represents 6- to 10-membered aromatic hydrocarbon ring, which may be substituted by halogen atom, hydroxyl, amino, C1-6 alkyl, C2-C3 alkenyl, C2-C3 alkynyl or thiophenyl;
L represents oxygen atom, or a substituted or unsubstituted C2-C3 alkynyl;
Z represents cyanoalkyl, alkylcarbonylaminoalkyl, alkylaminocarbonyl, alkylaminoalkyl, a substituted or unsubstituted, C3-C6 cycloalkyl, a 5- to 6-membered saturated ring which contains at least one heteroatom selected from the group consisting of N, S and O, or an 8-to 10-membered partially unsaturated ring which contains at least one heteroatom selected from the group consisting of N, S and O;
when L is C2-C3 alkynyl, Z is alkylaminocarbonyl or alkylaminoalkyl; and
m is 0 or 1.

The compound or a salt thereof of the present invention is more preferably a compound represented by formula (1) or a salt thereof,
wherein:
Ring A represents a substituted or unsubstituted, saturated or unsaturated 8- to 10-membered N-containing bridged ring which contains at least one further heteroatom selected from the group consisting of N, S and O;
Ring B represents a substituted or unsubstituted, 5- to 6-membered saturated or unsaturated ring having at least one heteroatom selected from the group consisting of N, S, and O, a 6-membered aromatic hydrocarbon ring, C3-C6 cycloalkyl ring, C3-C6 cycloalkenyl or an 8- to 10-membered spiro ring, wherein the Ring B is fused with the pyrimidine ring to form a substituted or unsubstituted bicyclic ring;
n is 0 or 1;
X is O or S;
Y represents benzene or naphthalene, which may be substituted by halogen atom, hydroxyl, amino, C1-C6 alkyl, C2-C3 alkenyl, C2-C3 alkynyl or thiophenyl;
L represents oxygen atom, or a substituted or unsubstituted C2-C3 alkynyl;
Z represents cyanoalkyl, alkylcarbonylaminoalkyl, alkylaminocarbonyl, alkylaminoalkyl, a substituted or unsubstituted, C3-C6 cycloalkyl, a 5- to 6-membered saturated ring which contains at least one heteroatom selected from the group consisting of N, S and O, or an 8-to 10-membered partially unsaturated ring which contains at least one heteroatom selected from the group consisting of N, S and O;
when L is C2-C3 alkynyl, Z is alkylaminocarbonyl or alkylaminoalkyl; and
m is 1.

The compound or a salt thereof of the present invention is preferably a compound represented by formula (1) or a salt thereof,
wherein:
Ring A represents a substituted or unsubstituted, saturated or unsaturated 8- to 10-membered N-containing bridged ring which contains at least one further heteroatom selected from the group consisting of N, S and O;
Ring B represents a substituted or unsubstituted, 5- to 6-membered saturated or unsaturated ring having at least one heteroatom selected from the group consisting of N, S, and O, a 6-membered aromatic hydrocarbon ring, C3-C6 cycloalkyl ring, C3-C6 cycloalkenyl or an 8- to 10-membered spiro ring, wherein the Ring B is fused with the pyrimidine ring to form a substituted or unsubstituted bicyclic ring;
n is 0 or 1;
X is O or S;
Y represents a substituted or unsubstituted, 6- to 10-membered unsaturated monocyclic or bicyclic ring which contains at least one heteroatom selected from the group consisting of N, S and O, or 6- to 10-membered aromatic hydrocarbon ring;
L represents oxygen atom;
Z represents cyanoalkyl, alkylcarbonylaminoalkyl, alkylaminocarbonyl, alkylaminoalkyl, a substituted or unsubstituted, C3-C6 cycloalkyl, a 5 to 6 membered saturated ring which contains at least one heteroatom selected from the group consisting of N, S and O, or an 8- to 10-membered partially unsaturated ring which contains at least one heteroatom selected from the group consisting of N, S and O; and
m is 0 or 1.

The compound or a salt thereof of the present invention is preferably a compound represented by formula (1) or a salt thereof,
wherein:
Ring A represents a substituted or unsubstituted, saturated or unsaturated 8- to 10-membered N-containing bridged ring which contains at least one further heteroatom selected from the group consisting of N, S and O;
Ring B represents a substituted or unsubstituted, 5- to 6-membered saturated or unsaturated ring having at least one heteroatom selected from the group consisting of N, S, and O, a 6-membered aromatic hydrocarbon ring, C3-C6 cycloalkyl ring, C3-C6 cycloalkenyl or an 8- to 10-membered spiro ring, wherein the Ring B is fused with the pyrimidine ring to form a substituted or unsubstituted bicyclic ring;
n is 0 or 1;
X is O or S;
Y represents a substituted or unsubstituted, 6- to 10-membered unsaturated monocyclic or bicyclic ring which contains at least one heteroatom selected from the group consisting of N, S and O, or 6- to 10-membered aromatic hydrocarbon ring;
L represents oxygen atom;
Z represents a substituted or unsubstituted C3-C6 cycloalkyl;
wherein the ring in Z may be substituted by halogen atom, hydroxyl, cyano, C1-C6 alkyl, C1-C3 alkoxy, alkylcarbonylalkyl, hydroxyalkyl, dialkylamino, dialkylaminoalkyl, alkoxyalkyl, cyanoalkyl, or C1-C6 alkyl which is substituted by 5- to 6-membered saturated ring which contains at least one heteroatom selected from the group consisting of N, S, and O, and which may be further substituted by halogen atom; and
m is 1.

The compound or a salt thereof of the present invention is preferably a compound represented by formula (1) or a salt thereof,
wherein:
Ring A represents a substituted or unsubstituted, saturated or unsaturated 8- to 10-membered N-containing bridged ring which contains at least one further heteroatom selected from the group consisting of N, S and O;
Ring B represents a substituted or unsubstituted, 5- to 6-membered saturated or unsaturated ring having at least one heteroatom selected from the group consisting of N, S, and O, a 6-membered aromatic hydrocarbon ring, C3-C6 cycloalkyl ring, C3-C6 cycloalkenyl or an 8- to 10-membered spiro ring, wherein the Ring B is fused with the pyrimidine ring to form a substituted or unsubstituted bicyclic ring;
n is 0 or 1;
X is O or S;
Y represents a substituted or unsubstituted, 6- to 10-membered unsaturated monocyclic or bicyclic ring which contains at least one heteroatom selected from the group consisting of N, S and O, or 6- to 10-membered aromatic hydrocarbon ring;
L represents oxygen atom;
Z represents a substituted or unsubstituted, a 5- to 6-membered saturated ring which contains at least one heteroatom selected from the group consisting of N, S and O, or an 8- to 10-membered partially unsaturated ring which contains at least one heteroatom selected from the group consisting of N, S and O;
wherein the ring in Z may be substituted by halogen atom, hydroxyl, cyano, C1-C6 alkyl, C1-C3 alkoxy, alkylcarbonylalkyl, hydroxyalkyl, dialkylamino, dialkylaminoalkyl, alkoxyalkyl, cyanoalkyl, or C1-C6 alkyl which is substituted by 5- to 6-membered saturated ring which contains at least one heteroatom selected from the group consisting of N, S, and O, and which may be further substituted by halogen atom; and
m is 0 or 1.

The compound or a salt thereof of the present invention is more preferably a compound represented by formula (1) or a salt thereof,
wherein:
Ring A is represented by the formula (3a) or (3b):

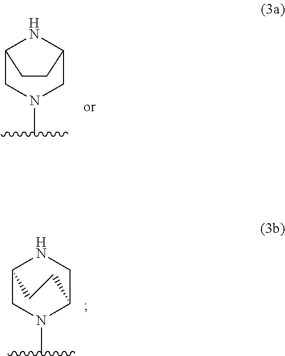 [SMILES: *N1CC2CCC(C1)N2 ; *N1C[C@@H]2CC[C@H]1CN2]

Ring B represents:
   (i) a 5- to 6-membered saturated or unsaturated ring which contains at least one heteroatom selected from the group consisting of N, S, and O,
   (ii) a 6- to 10-membered aromatic hydrocarbon ring,
wherein the Ring B is fused with the pyrimidine ring to form a substituted or unsubstituted bicyclic ring, and
wherein the Ring B in the bicyclic ring may be substituted by halogen atom, C1-C6 alkyl, alkylcarbonyl or 4- to 6-membered saturated monocyclic ring which contains at least one hetero atom selected from N, S, and O;
n is 0 or 1;
X is O or S;
Y represents a substituted or unsubstituted, 6- to 10-membered unsaturated monocyclic or bicyclic ring which contains at least one heteroatom selected from the group consisting of N, S and O, or 6- to 10-membered aromatic hydrocarbon ring;
L represents oxygen atom;
Z represents cyanoalkyl, alkylcarbonylaminoalkyl, alkylaminocarbonyl, alkylaminoalkyl, a substituted or unsubstituted, C3-C6 cycloalkyl, a 5 to 6 membered saturated ring which contains at least one heteroatom selected from the group consisting of N, S and O, or an 8- to 10-membered partially unsaturated ring which contains at least one heteroatom selected from the group consisting of N, S and O; and
m is 1.

The compound or a salt thereof of the present invention is more preferably a compound represented by formula (1) or a salt thereof,
wherein:
Ring A is represented by the formula (3a);

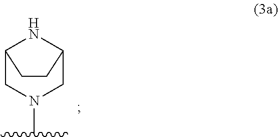 [SMILES: *N1CC2CCC(C1)N2]

Ring B represents benzene, piperidine, pyrrolidine, tetrahydro-2H-pyran, or 3,4-dihydro-2H-pyran,
wherein the Ring B is fused with the pyrimidine ring to form a substituted or unsubstituted bicyclic ring, and
wherein the Ring B in the bicyclic ring may be substituted by halogen atom, C1-C6 alkyl, alkylcarbonyl or 4- to 6-membered saturated monocyclic ring which contains at least one heteroatom selected from N, S, and O, and
when Ring B is pyrrolidine, n is 1 and X is O or S and when Ring B is not pyrrolidine, n is 0;
Y represents benzene or naphthalene, which may be substituted by halogen atom, hydroxyl, amino, C1-C6 alkyl, C2-C3 alkenyl or C2-C3 alkynyl;
L represents oxygen atom;
Z represents a substituted or unsubstituted C3-C6 cycloalkyl, wherein the ring in Z may be substituted by halogen atom, hydroxyl, cyano, C1-C6 alkyl, C1-C3 alkoxy, alkylcarbonylalkyl, hydroxyalkyl, dialkylamino, dialkylaminoalkyl, alkoxyalkyl, cyanoalkyl, or C1-C6 alkyl which is substituted by 5- to 6-membered saturated ring which contains at least one heteroatom selected from the group consisting of N, S, and O, and which may be further substituted by halogen atom; and
m is 1.

The compound or a salt thereof of the present invention is still more preferably a compound represented by formula (1) or a salt thereof,
wherein
Ring A is represented by the formula (3a)

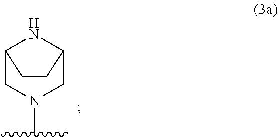 [SMILES: *N1CC2CCC(C1)N2]

Ring B represents benzene, piperidine, pyrrolidine, tetrahydro-2H-pyran, or 3,4-dihydro-2H-pyran, wherein the Ring B is fused with the pyrimidine ring to form a substituted or unsubstituted bicyclic ring, and
wherein the Ring B in the bicyclic ring may be substituted by halogen atom, C1-C6 alkyl, alkylcarbonyl or 4- to 6-membered saturated monocyclic ring which contains at least one heteroatom selected from N, S, and O, and
when Ring B is pyrrolidine, n is 1 and X is O or S and when Ring B is not pyrrolidine, n is 0;
Y represents benzene or naphthalene, which may be substituted by halogen atom, hydroxyl, amino, C1-C6 alkyl, C2-C3 alkenyl or C2-C3 alkynyl;
L represents oxygen atom;
Z represents a substituted or unsubstituted cyclopentane or cyclobutane,
wherein the ring in Z may be substituted by dimethylamino, dimethylaminomethyl, morpholinylmethyl, methylpyrrolidine or 3-fluoropyrrolidinemethyl; and
m is 1.

Examples of specific compounds of the present invention include, but are not limited to, the compounds produced in the Examples below.

Examples of preferable compounds of the present invention include the following:
(1) 4-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-5-bromonaphthalen-2-ol,
(2) 4-(4-(3,8-diazabicyclo[3.2.1]octan-3-yl)-2-((1-(((R)-3-fluoropyrrolidin-1-yl)methyl)cyclopropyl)methoxy)-5,8-dihydropyrido[3,4-d]pyrimidin-7(6H)-yl)-5-bromonaphthalen-2-ol,
(3) 4-(4-(3,8-diazabicyclo[3.2.1]octan-3-yl)-2-((1-(morpholinomethyl)cyclopropyl)methoxy)-5,8-dihydropyrido[3,4-d]pyrimidin-7(6H)-yl)-5-bromonaphthalen-2-ol,
(4) 4-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-5-methylnaphthalen-2-ol,
(5) 4-(4-(3,8-diazabicyclo[3.2.1]octan-3-yl)-2-((1-(((R)-3-fluoropyrrolidin-1-yl)methyl)cyclopropyl)methoxy)-5,8-dihydropyrido[3,4-d]pyrimidin-7(6H)-yl)-5-methylnaphthalen-2-ol,
(6) 4-(4-(3,8-diazabicyclo[3.2.1]octan-3-yl)-2-((1-(morpholinomethyl)cyclopropyl)methoxy)-5,8-dihydropyrido[3,4-d]pyrimidin-7(6H)-yl)-5-methylnaphthalen-2-ol,
(7) 4-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-5-iodonaphthalen-2-ol,
(8) 4-(4-(3,8-diazabicyclo[3.2.1]octan-3-yl)-2-((1-(((R)-3-fluoropyrrolidin-1-yl)methyl)cyclopropyl)methoxy)-5,8-dihydropyrido[3,4-d]pyrimidin-7(6H)-yl)-5-iodonaphthalen-2-ol,
(9) 4-(4-(3,8-diazabicyclo[3.2.1]octan-3-yl)-2-((1-(morpholinomethyl)cyclopropyl)methoxy)-5,8-dihydropyrido[3,4-d]pyrimidin-7(6H)-yl)-5-iodonaphthalen-2-ol,
(10) (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl) (8-ethynyl-3-hydroxynaphthalen-1-yl)methanone,
(11) (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl) (8-ethynyl-3-hydroxynaphthalen-1-yl)methanone,
(12) (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl) (3-hydroxy-8-iodonaphthalen-1-yl)methanone,
(13) (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl) (3-hydroxy-8-iodonaphthalen-1-yl)methanone,
(14) 4-(4-(3,8-diazabicyclo[3.2.1]octan-3-yl)-6-chloro-2-((1-((dimethylamino)methyl)cyclopropyl)methoxy)-8-fluoroquinazolin-7-yl)naphthalen-2-ol,
(15) 4-(4-(3,8-diazabicyclo[3.2.1]octan-3-yl)-2-((1-((dimethylamino)methyl)cyclopropyl)methoxy)-8-fluoroquinazolin-7-yl)naphthalen-2-ol,
(16) 4-(4-(3,8-diazabicyclo[3.2.1]octan-3-yl)-8-fluoro-2-(((S)-1-methylpyrrolidin-2-yl)methoxy)quinazolin-7-yl)naphthalen-2-ol,
(17) 4-(4-(3,8-diazabicyclo[3.2.1]octan-3-yl)-2-((cis-2-(dimethylamino)cyclobutyl)methoxy)-8-fluoroquinazolin-7-yl)naphthalen-2-ol,
(18) 4-(4-(3,8-diazabicyclo[3.2.1]octan-3-yl)-2-((1-((dimethylamino)methyl)cyclopropyl)methoxy)-6,8-difluoroquinazolin-7-yl)naphthalen-2-ol,
(19) 4-(4-(3,8-diazabicyclo[3.2.1]octan-3-yl)-2-((1-((dimethylamino)methyl)cyclopropyl)methoxy)-6-ethyl-8-fluoroquinazolin-7-yl)naphthalen-2-ol,
(20) 4-(4-((1S,4S)-2,5-diazabicyclo[2.2.2]octan-2-yl)-2-((1-((dimethylamino)methyl)cyclopropyl)methoxy)-5,8-dihydropyrido[3,4-d]pyrimidin-7(6H)-yl)-5-bromonaphthalen-2-ol,
(21) 4-(4-(3,8-diazabicyclo[3.2.1]octan-3-yl)-2-((1-((dimethylamino)methyl)-2,2-difluorocyclopropyl)methoxy)-5,8-dihydropyrido[3,4-d]pyrimidin-7(6H)-yl)-5-bromonaphthalen-2-ol,
(22) 1-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-8-bromoisoquinolin-3-amine,
(23) 4-(4-(3,8-diazabicyclo[3.2.1]octan-3-yl)-2-((1-((dimethylamino)methyl)cyclopropyl)methoxy)-7,8-dihydro-5H-pyrano[4,3-d]pyrimidin-7-yl)-5-ethynylnaphthalen-2-ol,
(24) 1-(1-(((4-(3,8-diazabicyclo[3.2.1]octan-3-yl)-7-(8-ethynylnaphthalen-1-yl)-5,6,7,8-tetrahydroquinazolin-2-yl)oxy)methyl)cyclopropyl)-N,N-dimethylmethanamine,
(25) 4-((1-(((4-(3,8-diazabicyclo[3.2.1]octan-3-yl)-7-(8-iodonaphthalen-1-yl)-5,6,7,8-tetrahydropyrido[3,4-d]pyrimidin-2-yl)oxy)methyl)cyclopropyl)methyl)morpholine,
(26) 4-(4-(3,8-diazabicyclo[3.2.1]octan-3-yl)-2-(((R)-1-methylpyrrolidin-2-yl)methoxy)-5,8-dihydropyrido[3,4-d]pyrimidin-7(6H)-yl)-5-bromonaphthalen-2-ol,
(27) 4-(4-(3,8-diazabicyclo[3.2.1]octan-3-yl)-2-(((2S,4R)-4-fluoro-1-methylpyrrolidin-2-yl)methoxy)-5,8-dihydropyrido[3,4-d]pyrimidin-7(6H)-yl)-5-bromonaphthalen-2-ol,
(28) 4-(4-((1S,4S)-2,5-diazabicyclo[2.2.2]octan-2-yl)-2-((1-(morpholinomethyl)cyclopropyl)methoxy)-5,8-dihydropyrido[3,4-d]pyrimidin-7(6H)-yl)-5-bromonaphthalen-2-ol,
(29) 1-(1-(((4-(3,8-diazabicyclo[3.2.1]octan-3-yl)-7-(8-bromonaphthalen-1-yl)-5,6,7,8-tetrahydropyrido[3,4-d]pyrimidin-2-yl)oxy)methyl)-2,2-difluorocyclopropyl)-N,N-dimethylmethanamine,
(30) 4-(4-(3,8-diazabicyclo[3.2.1]octan-3-yl)-2-((1-((dimethylamino)methyl)-2,2-dimethylcyclopropyl)methoxy)-5,8-dihydropyrido[3,4-d]pyrimidin-7(6H)-yl)-5-bromonaphthalen-2-ol,
(31) 4-(4-(3,8-diazabicyclo[3.2.1]octan-3-yl)-2-(((S)-1-methylpyrrolidin-2-yl)methoxy)-5,8-dihydropyrido[3,4-d]pyrimidin-7(6H)-yl)-5-bromonaphthalen-2-ol,
(32) 4-(4-(3,8-diazabicyclo[3.2.1]octan-3-yl)-2-(((2S,4R)-4-methoxy-1-methylpyrrolidin-2-yl)methoxy)-5,8-dihydropyrido[3,4-d]pyrimidin-7(6H)-yl)-5-bromonaphthalen-2-ol,
(33) 4-(4-(3,8-diazabicyclo[3.2.1]octan-3-yl)-2-((1-(morpholinomethyl)cyclopropyl)methoxy)-7,8-dihydro-5H-pyrano[4,3-d]pyrimidin-7-yl)-5-ethynylnaphthalen-2-ol,
(34) (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl) (3-hydroxy-8-vinylnaphthalen-1-yl)methanone,
(35) (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl) (3-hydroxy-8-vinylnaphthalen-1-yl)methanone,
(36) 1-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-8-ethynylisoquinolin-3-amine, and
(37) 4-(4-(3,8-diazabicyclo[3.2.1]octan-3-yl)-2-((1-((dimethylamino)methyl)cyclopropyl)methoxy)-7,8-dihydro-5H-pyrano[4,3-d]pyrimidin-7-yl)-5-bromonaphthalen-2-ol.

The following are details of the method for producing the compound of the present invention.

The compound represented by formula (1) of the present invention may be prepared from commercially available reagents using the synthetic methods and reaction schemes described herein, or using other reagents and conventional methods well known in the art, for example, through the following production methods or reaction steps described in the Examples.

However, the production methods are not limited to these methods and reaction scheme as long as a product of interest can be obtained. An intermediate product or a final product obtained in each step can be subjected to the subsequent step after, or without, isolation and purification by known separation and purification methods, such as concentration, vacuum concentration, crystallization, solvent extraction, reprecipitation, and chromatography.

To the reaction product obtained in each step and the starting material, a protecting group that can be easily converted to the functional group can be introduced if it is effective in each step, or so as to change the order of the steps. Examples of the protecting group used herein may be the protecting groups etc. used in the method disclosed in “Protective Groups in Organic Synthesis,” 5th edition, Greene and Wuts, John Wiley & Sons Inc., 2014. The protecting group may be appropriately selected according to the reaction conditions of each step. After introducing a protecting group and performing reaction, the protecting group is optionally removed to thus yield a desired compound.

Compounds of the formula (1) can be prepared in accordance with synthetic methods well known in the art.

According to a further aspect of the invention, provided is a process for preparing a compound of formula (1), or a tautomer, stereoisomer, pharmaceutically acceptable salt, or solvate thereof, which comprises following scheme:

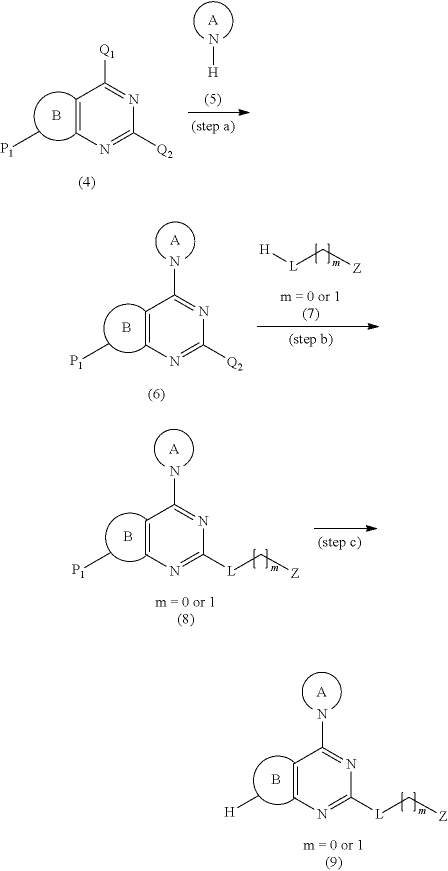

(wherein P₁ is protecting groups of heteroatom; Q₁ and Q₂ are leaving groups; and A, B, L, m, and Z are as defined above)

(Step a)

In this step, the compound of formula (4) is subjected to a coupling reaction with the compound of formula (5) to produce the compound of formula (6).

The compounds of formula (5) are either commercially available, or may be prepared using methods identical to or analogous to those described in the examples.

The process typically comprises, reacting a compound of formula (4) with a compound of formula (5) and suitable base in a suitable solvent at a suitable temperature. Example of the suitable base is N,N-diisopropylethylamine. Examples of the suitable solvents is N,N-dimethylacetamide.

The amount of a compound of formula (5) used herein is usually 1 to 100 moles, and preferably 1 to 10 moles, per mole of the compound represented by formula (4). The amount of the base used is usually 1 to 100 moles, and preferably 1 to 20 moles, per mole of the compound represented by formula (4).

The reaction temperature generally ranges from 0 to 100° C., preferably 0 to 60° C. The reaction time generally ranges from 5 minutes to 7 days, preferably 30 min to 4 days.

The thus-obtained compound of formula (6) can be subjected to the subsequent step after or without isolation or purification by known separation and purification means, such as concentration, vacuum concentration, crystallization, solvent extraction, reprecipitation, and chromatography.

(Step b)

In this step, the compound of formula (6) is subjected to a coupling reaction with the compound of formula (7) to produce the compound of formula (8).

The compounds of formula (7) are either commercially available, or may be prepared using methods identical to or analogous to those described in the examples.

The process typically comprises, reacting a compound of formula (6) with a compound of formula (7) and a suitable catalyst, a suitable base in a suitable solvent at a suitable temperature.

Examples of suitable catalysts are Ruphos Pd G3 or Ruphos Pd G4. Examples of suitable base are sodium carbonate, potassium carbonate, potassium phosphate and cesium carbonate. Examples of suitable solvents are tetrahydrofuran, 1,2-dimethoxyethane and 1,4-dioxane.

The amount of a compound of formula (7) used is usually 1 to 100 moles, and preferably 1 to 20 moles, per mole of the compound represented by formula (6). The amount of the catalyst used is usually 0.0001 to 1 moles, and preferably 0.001 to 0.5 moles, per mole of the compound represented by formula (6). The amount of the ligand used is usually 0.0001 to 4 moles, and preferably 0.001 to 2 moles, per mole of the compound represented by formula (6). The amount of the base used is usually 0.1 to 10 moles, and preferably 1 to 5 moles, per mole of the compound represented by formula (6).

The reaction temperature generally ranges from 0 to 200° C., preferably room temperature to 150° C. The reaction time generally ranges from 5 minutes to 7 days, preferably 30 min to 4 days.

The thus-obtained compound of formula (8) can be subjected to the subsequent step after or without isolation or purification by known separation and purification means, such as concentration, vacuum concentration, crystallization, solvent extraction, reprecipitation, and chromatography.

(Step c)

In this step, the compound of formula (8) is deprotected to produce the compound of formula (9).

Examples of the protecting group represented by P₁ in the compound of formula (8) include benzyloxycarbonyl (Cbz).

The process typically comprises, reacting a compound of formula (8) with a suitable catalyst in a suitable solvent at a suitable temperature and a suitable pressure under hydrogen atmosphere.

Examples of suitable catalysts are palladium on carbon, and palladium hydroxide on carbon. Examples of suitable solvents are methanol and ethanol.

The amount of the catalyst used is usually 1 to 300 wt %, and preferably 1 to 100 wt %, per mole of the compound represented by formula (8).

The reaction temperature generally ranges from 0 to 100° C., preferably room temperature to 60° C. The reaction pressure generally ranges from 1 to 20 atm, preferably 1 to 5 atm. The reaction time generally ranges from 5 minutes to 7 days, preferably 30 min to 4 days.

The thus-obtained compound of formula (9) can be subjected to the subsequent step after or without isolation or purification by known separation and purification means, such as concentration, vacuum concentration, crystallization, solvent extraction, reprecipitation, and chromatography.

(Step d) Buchwald Amination

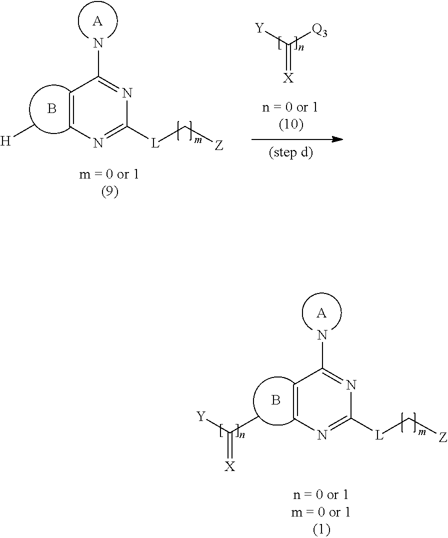

(wherein Q₃ is a halogen atom or a leaving group; A, B, L, m, n, X, Y, and Z are as defined above).

In this step, the compound of formula (9) is subjected to a coupling reaction with the compound of formula (10) to produce the compound of formula (1).

The compounds of formula (10) are either commercially available, or may be prepared using methods identical to or analogous to those described in the examples.

The process typically comprises, reacting a compound of formula (9) with a compound of formula (10) and a suitable catalyst, a suitable base in a suitable solvent at a suitable temperature.

Examples of suitable catalysts are PdCl₂dppf, RUPHOS Pd G4 and Pd₂dba₃ with suitable ligand (such as BINAP, Xantphos or Davephos). Examples of suitable base are NaOtBu, LHMDS, K₂CO₃ and Cs₂CO₃. Examples of suitable solvents are toluene, 1,4-dioxane and THF.

The amount of a compound of formula (10) used is usually 1 to 100 moles, and preferably 1 to 20 moles, per mole of the compound represented by formula (9). The amount of the catalyst used is usually 0.0001 to 1 moles, and preferably 0.001 to 0.6 moles, per mole of the compound represented by formula (9). The amount of the ligand used is usually 0.0001 to 4 moles, and preferably 0.001 to 2 moles, per mole of the compound represented by formula (9). The amount of the base used is usually 0.1 to 10 moles, and preferably 1 to 5 moles, per mole of the compound represented by formula (9).

The reaction temperature generally ranges from 0 to 200° C., preferably room temperature to 150° C. The reaction time generally ranges from 5 minutes to 7 days, preferably 30 min to 4 days.

The thus-obtained compound of formula (1) can be subjected to the subsequent step after or without isolation or purification by known separation and purification means, such as concentration, vacuum concentration, crystallization, solvent extraction, reprecipitation, and chromatography.

(Step e) Condensation Reaction

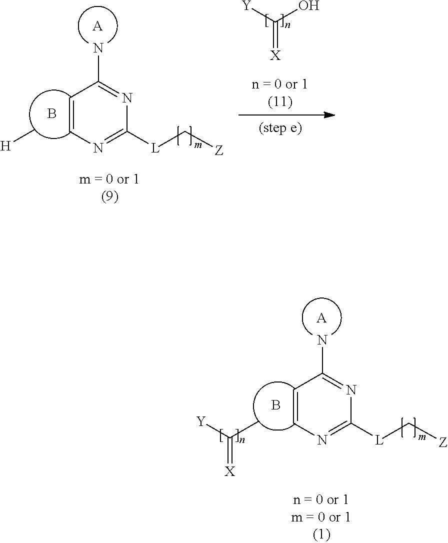

(wherein A, B, L, m, n, X, Y, Z are as defined above).

(Step e)

In this step, the compound of formula (9) is subjected to a condensation with the compound of formula (11) to produce the compound of formula (1).

The compounds of formula (11) are either commercially available, or may be prepared using methods identical to or analogous to those described in the examples.

The process typically comprises, reacting a compound of formula (9) with a compound of formula (11) and a suitable condensation reagent, a suitable base in a suitable solvent at a suitable temperature.

Examples of suitable condensation reagents are 1-(3-dimethylaminopropyl)-3-ethylcarbodiimide hydrochloride and 1-hydroxybenzotriazole or 1-[bis (dimethylamino)methylene]-1H-1,2,3-triazolo[4,5-b]pyridinium 3-oxide hexafluorophosphate. Examples of suitable base are triethylamine and N,N-diisopropylehtylamine. Examples of suitable solvents are N,N-dimethylformamide and tetrahydrofuran.

The amount of the compound of formula (11) used is usually 1 to 100 moles, and preferably 1 to 10 moles, per mole of the compound represented by formula (9). The amount of condensation reagents used is usually 1 to 100 moles, and preferably 1 to 10 moles, per mole of the compound represented by formula (9). The amount of the base used is usually 1 to 100 moles, and preferably 1 to 10 moles, per mole of the compound represented by formula (9).

The reaction temperature generally ranges from 0 to 200° C., preferably room temperature to 150° C. The reaction time generally ranges from 5 minutes to 7 days, preferably 30 min to 4 days.

The thus-obtained compound of formula (1) can be subjected to the subsequent step after or without isolation or purification by known separation and purification means, such as concentration, vacuum concentration, crystallization, solvent extraction, reprecipitation, and chromatography.

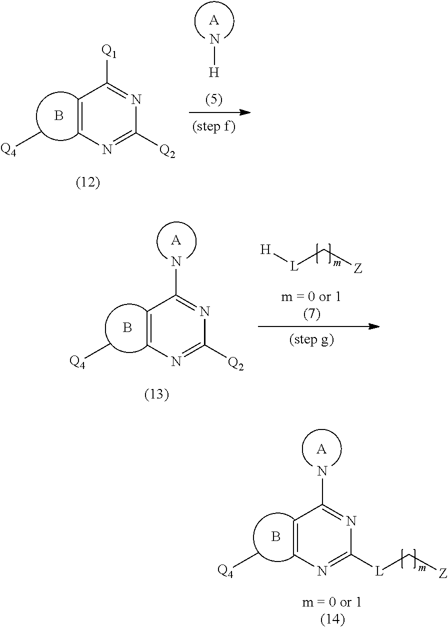

(wherein Q₁, Q₂, and Q₄ are halogen atom or leaving groups, A, B, L, m, and Z are as defined above)

(Step f)

In this step, the compound of formula (12) is subjected to a coupling reaction with the compound of formula (5) to produce the compound of formula (13).

The compounds of formula (5) are either commercially available, or may be prepared using methods identical to or analogous to those described in the examples.

The process typically comprises, reacting a compound of formula (12) with a compound of formula (5) and suitable base in a suitable solvent at a suitable temperature. Example of a suitable base is N,N-diisopropylethylamine. Example of suitable solvent is N,N-dimethylacetamide.

The amount of a compound of formula (5) used is usually 1 to 100 moles, and preferably 1 to 10 moles, per mole of the compound represented by formula (12). The amount of the base used is usually 1 to 100 moles, and preferably 1 to 20 moles, per mole of the compound represented by formula (12).

The reaction temperature generally ranges from 0 to 100° C., preferably 0 to 60° C. The reaction time generally ranges from 5 minutes to 7 days, preferably 30 min to 4 days.

The thus-obtained compound of formula (13) can be subjected to the subsequent step after or without isolation or purification by known separation and purification means, such as concentration, vacuum concentration, crystallization, solvent extraction, reprecipitation, and chromatography.

(Step g)

In this step, the compound of formula (13) is subjected to a coupling reaction with the compound of formula (7) to produce the compound of formula (14).

The compounds of formula (7) are either commercially available, or may be prepared using methods identical to or analogous to those described in the examples.

The process typically comprises, reacting a compound of formula (13) with a compound of formula (7) and a suitable catalyst, a suitable base in a suitable solvent at a suitable temperature.

Examples of suitable catalysts are Ruphos Pd G3 and Ruphos Pd G4. Examples of suitable base are sodium carbonate, potassium carbonate, potassium phosphate and cesium carbonate. Examples of suitable solvents are tetrahydrofuran, 1,2-dimethoxyethane and 1,4-dioxane.

The amount of a compound of formula (7) used is usually 1 to 100 moles, and preferably 1 to 20 moles, per mole of the compound represented by formula (13). The amount of the catalyst used is usually 0.0001 to 1 moles, and preferably 0.001 to 0.5 moles, per mole of the compound represented by formula (13). The amount of the ligand used is usually 0.0001 to 4 moles, and preferably 0.001 to 2 moles, per mole of the compound represented by formula (13). The amount of the base used is usually 0.1 to 10 moles, and preferably 1 to 5 moles, per mole of the compound represented by formula (13).

The reaction temperature generally ranges from 0 to 200° C., preferably room temperature to 150° C. The reaction time generally ranges from 5 minutes to 7 days, preferably 30 min to 4 days.

The thus-obtained compound of formula (14) can be subjected to the subsequent step after or without isolation or purification by known separation and purification means, such as concentration, vacuum concentration, crystallization, solvent extraction, reprecipitation, and chromatography.

Suzuki-Miyaura Coupling

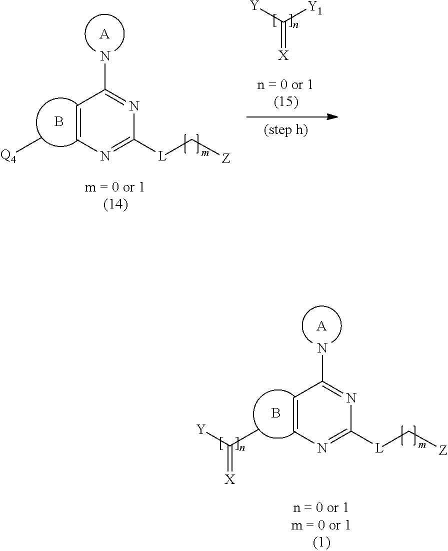

(wherein T₁ represents a metal or metaloid residue (such as boronic acid or pinacol boronate), and A, B, L, m, n, X, Y, and Z are as defined above).

(Step h)

In this step, the compound of formula (14) is subjected to a coupling reaction with the compound of formula (15) to produce the compound of formula (1).

The compounds of formula (15) are either commercially available, or may be prepared using methods identical to or analogous to those described in the examples.

The process typically comprises, reacting a compound of formula (14) with the compound of formula (15) and a suitable catalyst in a suitable solvent at a suitable temperature. Examples of suitable catalysts are [1,1′-bis (diphenylphosphino)ferrocene]palladium(II) dichloride, tetrakistriphenylphosphine palladium and tris(dibenzylideneacetone)dipalladium(0) with a suitable ligand (such as triphenylphosphine, tri-tert-butylphosphine, 2-dicyclohexylphosphino-2′,4′,6′-triisopropylbiphenyl).

Examples of suitable base are sodium carbonate, potassium carbonate and potassium phosphate. Examples of suitable solvents are tetrahydrofuran, 1,2-dimethoxyethane and 1,4-dioxane with water. The amount of an amine of formula (VII) used is usually 1 to 10 moles, and preferably 1 to 5 moles, per mole of the compound represented by formula (14).

The amount of the catalyst used is usually 0.0001 to 1 moles, and preferably 0.001 to 0.5 moles, per mole of the compound represented by formula (14). The amount of the ligand used is usually 0.0001 to 4 moles, and preferably 0.001 to 2 moles, per mole of the compound represented by formula (14). The amount of the base used is usually 0.1 to 10 moles, and preferably 1 to 5 moles, per mole of the compound represented by formula (14).

The amount of the base used is generally 1 to 100 moles, preferably 1 to 10 moles, per mole of the compound represented by formula (14). The reaction temperature generally ranges from 0 to 200° C., preferably room temperature to 150° C. The reaction time generally ranges from 5 minutes to 7 days, preferably 30 minutes to 4 days.

The thus-obtained compound of formula (1) can be subjected to the subsequent step after or without isolation or purification by known separation and purification means, such as concentration, vacuum concentration, crystallization, solvent extraction, reprecipitation, and chromatography.

When the compound of the present invention has isomers such as optical isomers, stereoisomers, rotational isomers, and tautomers, any of the isomers and mixtures thereof are included within the scope of the compound of the present invention unless otherwise specified. For example, when the compound of the present invention has optical isomers, racemic mixtures and the optical isomers separated from a racemic mixture are also included within the scope of the compound of the present invention unless otherwise specified.

The compound or a salt thereof of the present invention may be in the form of amorphous or crystals. Single crystals and polymorphic mixtures are included within the scope of the compound or a salt thereof of the present invention. Such crystals can be produced by crystallization according to a crystallization method known in the art. The compound or a salt thereof of the present invention may be a solvate (e.g., a hydrate) or a non-solvate. Any of such forms are included within the scope of the compound or a salt thereof of the present invention. Compounds labeled with an isotope (e.g., ²H, ³H, ¹³C, ¹⁴C, ³⁵S, ¹²⁵I) are also included within the scope of the compound or a salt thereof of the present invention.

The salts of the compound of the present invention refer to any pharmaceutically acceptable salts; examples include base addition salts and acid addition salts.

In yet another embodiment, the present invention provides a medicament containing the compound of the present invention or a salt thereof as an active ingredient. Furthermore, the present invention relates to use of the compound of the present invention or a salt thereof for the manufacture of a medicament. Further, the present invention provides the use as medicaments of the compound of the present invention or a salt thereof. Further, provided is the compound of the present invention or a salt thereof for use as a medicament.

In yet another embodiment, the present invention provides a pharmaceutical composition comprising the compound of the present invention or a salt thereof and a pharmaceutically acceptable carrier.

In a preferred embodiment, the medicament or pharmaceutical composition is a therapeutic agent for the KRAS-related diseases, in a more preferred embodiment, the medicament or pharmaceutical composition is an antitumor agent.

As used herein, KRAS-related diseases refer to a “KRAS G12D-associated disease or disorder”. The “KRAS G12D-associated disease or disorder” as used herein refers to diseases or disorders associated with or mediated by or having a KRAS G12D mutation. A non-limiting example of a KRAS G12D-associated disease or disorder is a KRAS G12D-associated cancer.

“KRAS G12D” refers to a mutant form of a mammalian KRAS protein that contains an amino acid substitution of an aspartic acid for a glycine at amino acid position 12. The assignment of amino acid codon and residue positions for human KRAS is based on the amino acid sequence identified by, for example, GenPept ID NP_004976.

As used herein, a “KRAS G12D inhibitor” refers to compounds of the present invention that are represented by formula (1) as described herein. These compounds are capable of negatively modulating or inhibiting all or a portion of the enzymatic activity of KRAS G12D. The KRAS G12D inhibitors of the present invention bind to KRAS G12D by forming a ionic interaction with the aspartic acid at position 12 of inactive KRAS (GDP), thus preventing conversion of inactive KRAS (GDP) to active KRAS (GTP) and inhibiting downstream signaling.

In yet another embodiment, the present invention comprises administering an effective amount of the compound of the present invention or a salt thereof to a subject to provide a KRAS G12D mutation activity suppression method. Further, the present invention comprises administering a therapeutically effective amount of the compound of the present invention or a salt thereof to a subject to provide a method of treating KRAS-related diseases. In a preferred embodiment, a method of treating KRAS-related diseases is a method of treating tumors. In the treatment method, the subjects include human or non-human animal in need of the method.

As used herein, the “effective amount” of the compound according to an embodiment of the present invention refers to an amount of the compound which is sufficient to achieve a biological response or therapeutic response of a subject, such as causing reduction or prevention of an activity of enzyme or protein; or improving a symptom, alleviating a medical state, delaying or retarding progression of disorder, or preventing a disease (therapeutically effective amount).

As used herein, the “subject” includes a mammal and a nonmammal. Examples of a mammal include, but not limited to, a human, a chimpanzee, an anthropoid, a monkey, a cow, a horse, a sheep, a goat, a pig, a rabbit, a dog, a cat, a rat, a mouse, a Cavia porcellus, a hedgehog, a kangaroo, a mole, a boar, a bear, a tiger and a lion. Examples of a nonmammal include, but not limited to, birds, fishes and reptiles. In one embodiment, the subject is a human, and may be a human who has been diagnosed to need a treatment for the symptom, the medical state or disease as disclosed herein.

In some embodiments, the subject has experienced and/or exhibited at least one symptom of the disease or disorder to be treated and/or prevented. In some embodiments, the subject has been identified or diagnosed as having a cancer having a KRAS G12D mutation. In some embodiments, the subject has a tumor that is positive for a KRAS G12D mutation.

In one embodiment, a medicament, a pharmaceutical composition or a pharmaceutical preparation comprising the compound or pharmaceutically acceptable salt thereof of the present invention may be provided. In another embodiment, an anti-tumor agent comprising the compound or pharmaceutically acceptable salt thereof of the present invention as an active ingredient may be provided.

The compound or a salt thereof of the present invention also encompasses prodrugs thereof. A prodrug refers to a compound that can be converted to the compound or a salt thereof of the present invention through a reaction with an enzyme, gastric acid, or the like under physiological conditions in vivo, i.e., a compound that can be converted to the compound or a salt thereof of the present invention by enzymatic oxidation, reduction, hydrolysis, or the like; or a compound that can be converted to the compound or a salt thereof of the present invention by hydrolysis or the like with gastric acid or the like.

Further, the prodrug may be compounds that can be converted to the compound or a salt thereof of the present invention under physiological conditions, such as those described in Iyakuhin no Kaihatsu, “Development of Pharmaceuticals,” Vol. 7, Molecular Design, published in 1990 by Hirokawa Shoten Co., pp. 163-198.

When the compound or a salt thereof of the present invention is used as a pharmaceutical preparation, a pharmaceutical carrier can be added, if required, thereby forming a suitable dosage form according to prevention and treatment purposes. Examples of the dosage form include oral preparations, injections, suppositories, ointments, inhalations, patches, and the like. Such dosage forms can be formed by methods conventionally known to a person skilled in the art.

As the pharmaceutical acceptable carrier, various conventional organic or inorganic carrier materials used as preparation materials may be blended as an excipient, binder, disintegrant, lubricant, or colorant in solid preparations; or as a solvent, solubilizing agent, suspending agent, isotonizing agent, buffer, or soothing agent in liquid preparations. Moreover, pharmaceutical preparation additives, such as antiseptics, antioxidants, colorants, sweeteners, and stabilizers, may also be used, if required.

In one embodiment, a medicament, a pharmaceutical composition or a pharmaceutical preparation for oral administration or an oral solid preparation comprising the compound or pharmaceutically acceptable salt thereof of the present invention may be provided. In other embodiment, an anti-tumor agent for oral administration comprising the compound or pharmaceutically acceptable salt thereof of the present invention as an active ingredient may be provided.

Oral solid preparations or a medicament, a pharmaceutical composition, an anti-tumor agent or a pharmaceutical preparation for oral administration are prepared as follows. After an excipient is added optionally with a binder, disintegrant, lubricant, colorant, taste-masking or flavoring agent, etc. to the compound or a salt thereof of the present invention, the resulting mixture is formulated into tablets, coated tablets, granules, powders, capsules, or the like by ordinary methods.

Oral solid preparations are prepared as follows. After an excipient is added optionally with a binder, disintegrant, lubricant, colorant, taste-masking or flavoring agent, etc. to the compound or a salt thereof of the present invention, the resulting mixture is formulated into tablets, coated tablets, granules, powders, capsules, or the like by ordinary methods.

When an injection agent is prepared, a pH regulator, a buffer, a stabilizer, an isotonizing agent, a local anesthetic, and the like may be added to the compound of the present invention; and the mixture may be formulated into a subcutaneous, intramuscular, or intravenous injection according to an ordinary method.

The amount of the compound of the present invention to be incorporated in each of such dosage unit forms depends on the condition of the patient to whom the compound is administered, the dosage form, etc. In general, for an oral agent, the amount of the compound is preferably about 0.05 to 1000 mg per dosage unit form. For an injection, the amount of the compound is preferably about 0.01 to 500 mg per dosage unit form, and for a suppository, the amount of the compound is preferably about 1 to 1000 mg per dosage unit form.

Further, the daily dose of the medicine in such a dosage form varies depending on the condition, body weight, age, sex, etc. of the patient, and cannot be unconditionally determined. For example, the daily dose for an adult (body weight: 50 kg) of the compound of the present invention may be generally about 0.05 to 5000 mg, and preferably 0.1 to 1000 mg.

The effective amount or administration regimen of the compound of the formula (1) of the present invention or a pharmaceutically acceptable salt thereof administered to the above subject can be suitably determined by a person skilled in the art depending on, for example, species, symptom, weight, age, or sex, of the subject. For example, when the subject is an adult human, it is usually administered at 0.05 to 5000 mg, and preferably 0.1 to 1000 mg per day in terms of the amount of the compound of the formula (1) of the present invention.

The compound or a salt thereof of the present invention has excellent KRAS inhibitory activity against KRAS G12D mutation-positive cancer cells, and also has excellent selectivity for KRAS G12D mutation than wild-type KRAS normal cells. Therefore, the compound or a salt thereof of the present invention is useful as an antitumor agent against KRAS G12D mutation-positive cancer cells, and has the advantage of fewer side effects.

Due to its excellent KRAS G12D inhibitory activity, the compound or a salt thereof of the present invention inhibits the KRAS function and is useful as a pharmaceutical preparation for preventing and treating KRAS-associated signaling-related diseases.

In one embodiment, use of a compound or pharmaceutically acceptable salt thereof of the present invention for manufacturing a pharmaceutical composition may be provided. In one embodiment, use of a compound or pharmaceutically acceptable salt thereof of the present invention for manufacturing an anti-tumor agent may be provided. In one embodiment, use of a compound or pharmaceutically acceptable salt thereof of the present invention for manufacturing an anti-tumor agent for oral administration may be provided. In one embodiment, a compound or pharmaceutically acceptable salt thereof of the present invention for use as medicament may be provided.

In one embodiment, a compound or pharmaceutically acceptable salt thereof of the present invention for use in the prevention and/or treatment of tumor may be provided. In one embodiment, a compound or pharmaceutically acceptable salt thereof of the present invention for use in the prevention and/or treatment of tumor by oral administration may be provided.

In one embodiment, there is provided a method for preventing and/or treating tumor, comprising administrating a therapeutically effective amount of the compound or pharmaceutically acceptable salt thereof of the present invention to a subject in need thereof. In one embodiment, an antitumor agent which is administered to a subject in need thereof in combination with a pharmaceutically effective amount of one or more other antitumor drugs may be provided.

In one embodiment, there is provided a method for preventing and/or treating tumor, comprising administrating a therapeutically effective amount of the compound or pharmaceutically acceptable salt thereof of the present invention to a subject in need thereof. In one embodiment, an antitumor agent which is administered to a subject in need thereof in combination with a pharmaceutically effective amount of one or more other antitumor drugs may be provided.

In terms of RAS-associated signaling in the KRAS-associated signaling-related diseases, KRAS is involved in various signaling transduction as RAS-associated signaling; KRAS mainly activates, but is not limited to, RAF, PI3K, RAL-GEF, and the like. Examples of the diseases include diseases whose incidence can be reduced, and whose symptoms can be remitted, relieved, and/or completely cured by deleting, suppressing, and/or inhibiting their functions.

Examples of such diseases include, but are not limited to, tumors, cancers, autoimmune diseases, macroglobulinemia, and the like. Cancer or tumor, in accordance with the present disclosure includes, but is not limited to, glandular tumors, carcinoid tumors, undifferentiated carcinomas, angiosarcoma, adenocarcinoma, sarcoma, neuroma, gastrointestinal cancers (e.g., colorectal cancers (“CRC”) including colon cancer and rectal cancer, biliary cancers including gall bladder cancer and bile duct cancer, anal cancer, esophageal cancer, gastric (stomach) cancer, gastrointestinal carcinoid tumor(s), gastrointestinal stromal tumor (s) (“GIST”), liver cancer, duodenal cancer and small intestine cancer), digestive organ cancer, lung cancers (e.g., non-small cell lung cancer (“NSCLC”), squamous-cell lung carcinoma, large-cell lung carcinoma, small cell lung carcinoma, mesothelioma and other lung cancers such as bronchial tumors and pleuropulmonary blastoma), urological cancers (e.g., kidney (renal) cancer, transitional cell cancer (“TCC”) of kidney, TCC of the renal pelvis and ureter (“PDQ”), bladder cancer, urethral cancer and prostate cancer), head and neck cancers (e.g., eye cancer, retinoblastoma, intraocular melanoma, hypopharyngeal cancer, pharyngeal cancer, laryngeal cancer, laryngeal papillomatosis, metastatic squamous neck cancer with occult primary, oral (mouth) cancer, lip cancer, throat cancer, oropharyngeal cancer, esthesioneuroblastoma, nasal cavity and paranasal sinus cancer, nasopharyngeal cancer, and salivary gland cancer), endocrine cancers (e.g., thyroid cancer, parathyroid cancer, multiple endocrine neoplasia syndromes, thymoma and thymic carcinoma, pancreatic cancers including pancreatic ductal adenocarcinoma (“PDAC”), pancreatic neuroendocrine tumors and islet cell tumors), breast cancers (extrahepatic ductal carcinoma in situ (“DCIS”), lobular carcinoma in situ (“LCIS”), triple negative breast cancer, and inflammatory breast cancer), male and female reproductive and/or genital cancers (e.g., cervical cancer, ovarian cancer, endometrial cancer, uterine sarcoma, uterine cancer, vaginal cancer, vulvar cancer, gestational trophoblastic tumor (“GTD”), extragonadal germ cell tumor, extracranial germ cell tumor, germ cell tumor, testicular cancer and penile cancer), brain and nervous system cancers (e.g., astrocytomas, brain stem glioma, brain tumor, craniopharyngioma, central nervous system (“CNS”) cancer, chordomas, ependymoma, embryonal tumors, neuroblastoma, paraganglioma and atypical teratoid), skin cancers (e.g., basal cell carcinoma (“BCC”), squamous cell skin carcinoma (“SCC”), Merkel cell carcinoma and melanoma), tissue and bone cancers (e.g., soft-tissue sarcoma, rhabdomyosarcoma, fibrous histiocytoma of bone, Ewing sarcoma, malignant fibrous histiocytoma of bone (“MFH”), osteosarcoma and chondrosarcoma), cardiovascular cancers (e.g., heart cancer and cardiac tumors), appendix cancers, childhood and adolescent cancers (e.g., adrenocortical carcinoma childhood, midline tract carcinoma, hepatocellular carcinoma (“HCC”), hepatoblastoma and Wilms' tumor) and viral-induced cancers (e.g., HHV-8 related cancers (Kaposi sarcoma) and HIV/AIDS related cancers). In some embodiments, the cancer is lung cancer, pancreatic cancer, rectal cancer, colon cancer, or colorectal cancer. In one embodiment, squamous carcinoma is a cancer of uterine cervix, tarsus, conjunctiva, vagina, lung, oral cavity, skin, bladder, tongue, larynx or esophagus. In one embodiment, adenocarcinoma is a cancer of prostate, small intestine, endometrium, uterine cervix, large intestine, lung, pancreas, esophagus, rectum, uterus, stomach, breast or ovary. In one embodiment, tumor is rectal cancer, colon cancer, colorectal cancer, pancreatic cancer, lung cancer, breast cancer, leukemia or uterine cancer. In one embodiment, a subject suffering from any of the diseases selected from the above does not have to have KRAS G12D mutant protein. In a preferred embodiment, a subject suffering from any of the diseases selected from the above has KRAS G12D mutant protein.

Cancer, in accordance with the present disclosure also includes, but is not limited to, hematological and plasma cell malignancies and hematopoietic tumors (e.g., cancers that affect blood, bone marrow and/or lymph nodes) such as multiple myeloma, leukemias and lymphomas, myelodysplastic syndromes and myeloproliferative disorders. Leukemias include, without limitation, acute lymphoblastic leukemia (“ALL”), acute myelogenous (myeloid) leukemia (“AML”), chronic lymphocytic leukemia (“CLL”), chronic myelogenous leukemia (“CML”), acute monocytic leukemia (“AMoL”), hairy cell leukemia, and/or other leukemias. Lymphomas include, without limitation, Hodgkin's lymphoma and non-Hodgkin's lymphoma (“NHL”). In some embodiments, NHL is B-cell lymphomas and/or T-cell lymphomas. In some embodiments, NHL includes, without limitation, diffuse large B-cell lymphoma (“DLBCL”), small lymphocytic lymphoma (“SLL”), chronic lymphocytic leukemia (“CLL”), mantle cell lymphoma (“MCL”), Burkitt's lymphoma, cutaneous T-cell lymphoma including mycosis fungoides and Sézary syndrome, AIDS-related lymphoma, follicular lymphoma, lymphoplasmacytic lymphoma (Waldenstrom's macroglobulinemia (“WM”)), primary central nervous system (CNS) lymphoma and/or other lymphomas.

In one embodiment, an antitumor agent comprising a compound or a pharmaceutically acceptable salt thereof of the present invention, and one or more other antitumor agents as an active ingredient may be provided. In one embodiment, an antitumor agent comprising a compound or a pharmaceutically acceptable salt thereof of the present invention as an active ingredient, which is administered in combination with one or more other antitumor agents may be provided.

In one embodiment, use of the compound of the present invention or a salt thereof and one or more other antitumor agents for the manufacture of an antitumor agent may be provided. In one embodiment, use of the compound of the present invention or a salt thereof for the manufacture of an antitumor agent, which is administered in combination with one or more other antitumor agents may be provided.

In one embodiment, the combination of a compound of the present invention or a salt thereof and one or more other antitumor agents for use in the treatment of tumors may be provided. In one embodiment, the compound or pharmaceutically acceptable salt thereof of the present invention for use in the treatment of tumor, which is administered in combination with one or more other antitumor agents may be provided.

In one embodiment, a method for treating tumor, comprising administrating a therapeutically effective amount of the compound or pharmaceutically acceptable salt thereof of the present invention, and one or more other antitumor agents to a subject in need thereof may be provided.

In one embodiment, a method for treating tumor, comprising administrating a therapeutically effective amount of the compound or pharmaceutically acceptable salt thereof of the present invention, which is administered in combination with one or more other antitumor agents to a subject in need thereof may be provided.

The compound or pharmaceutically acceptable salt thereof of the present invention can be used to treat cancer in combination with one or more other antitumor agents. In other words, a single compound or pharmaceutically acceptable salt thereof of the present invention or more than one compound or pharmaceutically acceptable salt thereof of the present invention may be used in combination with a single other antitumor agent or more than one other antitumor agents.

As used herein, an “other antitumor agent” can be any pharmaceutically active agent (or pharmaceutically acceptable salt thereof) that is active in the body and that is different from the compound or pharmaceutically acceptable salt thereof of the present invention. The other antitumor agents include prodrugs, free-acid, free-base and pharmaceutically acceptable salts of the additional active agents. Generally any suitable other antitumor agent, including chemotherapeutic agents or therapeutic antibodies, may be used in any combination with a compound or pharmaceutically acceptable salt thereof of the present invention in a single dosage formulation (e.g., a fixed dose drug combination) or in one or more separate dosage formulations which allow for concurrent or sequential administration of the pharmaceutically active agents (co-administration of the separate active agents) to subjects.

In certain embodiments, a compound or pharmaceutically acceptable salt thereof of the present invention and an other antitumor agent are administered a few minutes apart, or a few hours apart, or a few days apart. In addition, the compound or pharmaceutically acceptable salt thereof of the present invention can be administered in combination with radiation therapy, hormone therapy, targeted therapy, surgery or immunotherapy. In one embodiment, the one or more other antitumor agents are included in a pharmaceutical composition as described above.

In one embodiment, the other antitumor agent(s) is an additional anti-cancer agent (also known as an antineoplastic agent). As used herein, an “anti-cancer agent” is any pharmaceutically active agent (or pharmaceutically active salt thereof) that is active in the body against cancer. Examples of anti-cancer agents include chemotherapeutic agents (e.g., cytotoxic agents), immunotherapeutic agents, hormonal and anti-hormonal agents, targeted therapy agents, and anti-angiogenesis agents. Many anti-cancer agents can be classified within one or more of these groups. While certain anti-cancer agents have been categorized within a specific group(s) or subgroup(s) herein, many of these agents can also be listed within one or more other group(s) or subgroup(s), as would be presently understood in the art. It is to be understood that the classification herein of a particular agent into a particular group is not intended to be limiting. Many anti-cancer agents are presently known in the art and can be used in combination with the compound or pharmaceutically acceptable salt thereof of the present invention.

Further, an agent can be an agonist, antagonist, allosteric modulator, toxin or, more generally, may act to inhibit or stimulate its target (e.g., receptor or enzyme activation or inhibition). For example, suitable for use are one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”.

In an embodiment, the additional anti-cancer agent is a chemotherapeutic agent, an immunotherapeutic agent, a hormonal agent, an anti-hormonal agent, a targeted therapy agent, or an anti-angiogenesis agent (or angiogenesis inhibitor). In an embodiment, the additional anti-cancer agent is selected from the group consisting of a chemotherapeutic agent, a mitotic inhibitor, a plant alkaloid, an alkylating agent, an anti-metabolite, a platinum analog, an enzyme, a topoisomerase inhibitor, a retinoid, an aziridine, an antibiotic, a hormonal agent, an anti-hormonal agent, an anti-estrogen, an anti-androgen, an anti-adrenal, an androgen, a targeted therapy agent, an immunotherapeutic agent, a biological response modifier, a cytokine inhibitor, a tumor vaccine, a monoclonal antibody, an immune checkpoint inhibitor, an anti-PD-1 agent, an anti-PD-L1 agent, a colony-stimulating factor, an immunomodulator, an immunomodulatory imide (IMiD), an anti-CTLA4 agent, an anti-LAGl agent, an anti-OX40 agent, a GITR agonist, a CAR-T cell, a BiTE, a signal transduction inhibitor, a growth factor inhibitor, a tyrosine kinase inhibitor, an EGFR inhibitor, a histone deacetylase (HDAC) inhibitor, a proteasome inhibitor, a cell-cycle inhibitor, an anti-angiogenesis agent, a matrix-metalloproteinase (MMP) inhibitor, a hepatocyte growth factor inhibitor, a TOR inhibitor, a KDR inhibitor, a VEGF inhibitor, a HIF-1α inhibitor a HIF-2α inhibitor, a fibroblast growth factor (FGF) inhibitor, a RAF inhibitor, a MEK inhibitor, an ERK inhibitor, a PI3K inhibitor, an AKT inhibitor, an MCL-1 inhibitor, a BCL-2 inhibitor, an SHP2 inhibitor, a HER-2 inhibitor, a BRAF-inhibitor, a gene expression modulator, an autophagy inhibitor, an apoptosis inducer, an antiproliferative agent, and a glycolysis inhibitor.

In one embodiment, the additional anti-cancer agent(s) is a chemotherapeutic agent. Non-limiting examples of chemotherapeutic agents include mitotic inhibitors and plant alkaloids, alkylating agents, anti-metabolites, platinum analogs, enzymes, topoisomerase inhibitors, retinoids, aziridines, and antibiotics.

Non-limiting examples of mitotic inhibitors and plant alkaloids include taxanes such as cabazitaxel, docetaxel, larotaxel, ortataxel, paclitaxel, and tesetaxel; demecolcine; epothilone; eribulin; etoposide (VP-16); etoposide phosphate; navelbine; noscapine; teniposide; thaliblastine; vinblastine; vincristine; vindesine; vinflunine; and vinorelbine.

Non-limiting examples of alkylating agents include nitrogen mustards such as chlorambucil, chlornaphazine, cholophosphamide, cytophosphane, estramustine, ifosfamide, mannomustine, mechlorethamine, mechlorethamine oxide hydrochloride, melphalan, novembichin, phenesterine, prednimustine, tris(2-chloroethyl)amine, trofosfamide, and uracil mustard; alkyl sulfonates such as busulfan, improsulfan, and piposulfan; nitrosoureas such as carmustine, chlorozotocin, fotemustine, lomustine, nimustine, ranimustine, streptozotocin, and TA-07; ethylenimines and methylamelamines such as altretamine, thiotepa, triethylenemelamine, triethylenethiophosphaoramide, trietylenephosphoramide, and trimethylolomelamine; ambamustine; bendamustine; dacarbazine; etoglucid; irofulven; mafosfamide; mitobronitol; mitolactol; pipobroman; procarbazine; temozolomide; treosulfan; and triaziquone.

Non-limiting examples of anti-metabolites include folic acid analogues such as aminopterin, denopterin, edatrexate, methotrexate, pteropterin, raltitrexed, and trimetrexate; purine analogs such as 6-mercaptopurine, 6-thioguanine, fludarabine, forodesine, thiamiprine, and thioguanine; pyrimidine analogs such as 5-fluorouracil (5-FU), 6-azauridine, ancitabine, azacytidine, capecitabine, carmofur, cytarabine, decitabine, dideoxyuridine, doxifiuridine, doxifluridine, enocitabine, floxuridine, galocitabine, gemcitabine, and sapacitabine; 3-aminopyridine-2-carboxaldehyde thiosemicarbazone; broxuridine; cladribine; cyclophosphamide; cytarabine; emitefur; hydroxyurea; mercaptopurine; nelarabine; pemetrexed; pentostatin; tegafur; and troxacitabine.

Non-limiting examples of platinum analogs include carboplatin, cisplatin, dicycloplatin, heptaplatin, lobaplatin, nedaplatin, oxaliplatin, satraplatin, and triplatin tetranitrate.

Non-limiting examples of enzymes include asparaginase and pegaspargase.

Non-limiting examples of topoisomerase inhibitors include acridine carboxamide, amonafide, amsacrine, belotecan, elliptinium acetate, exatecan, indolocarbazole, irinotecan, lurtotecan, mitoxantrone, razoxane, rubitecan, SN-38, sobuzoxane, and topotecan.

Non-limiting examples of retinoids include alitretinoin, bexarotene, fenretinide, isotretinoin, liarozole, RII retinamide, and tretinoin.

Non-limiting examples of aziridines include benzodopa, carboquone, meturedopa, and uredopa.

Non-limiting examples of antibiotics include intercalating antibiotics; anthracenediones; anthracycline antibiotics such as aclarubicin, amrubicin, daunomycin, daunorubicin, doxorubicin, epirubicin, idarubicin, menogaril, nogalamycin, pirarubicin, and valrubicin; 6-diazo-5-oxo-L-norleucine; aclacinomysins; actinomycin; authramycin; azaserine; bleomycins; cactinomycin; calicheamicin; carabicin; carminomycin; carzinophilin; chromomycins; dactinomycin; detorubicin; esorubicin; esperamicins; geldanamycin; marcellomycin; mitomycins; mitomycin C; mycophenolic acid; olivomycins; novantrone; peplomycin; porfiromycin; potfiromycin; puromycin; quelamycin; rebeccamycin; rodorubicin; streptonigrin; streptozocin; tanespimycin; tubercidin; ubenimex; zinostatin; zinostatin stimalamer; and zorubicin.

In one embodiment, the additional anti-cancer agent(s) is a hormonal and/or anti-hormonal agent (i.e., hormone therapy). Non-limiting examples of hormonal and anti-hormonal agents include anti-androgens such as abiraterone, apalutamide, bicalutamide, darolutamide, enzalutamide, flutamide, goserelin, leuprolide, and nilutamide; anti-estrogens such as 4-hydroxy tamoxifen, aromatase inhibiting 4(5)-imidazoles, EM-800, fosfestrol, fulvestrant, keoxifene, LY 117018, onapristone, raloxifene, tamoxifen, toremifene, and trioxifene; anti-adrenals such as aminoglutethimide, dexaminoglutethimide, mitotane, and trilostane; androgens such as calusterone, dromostanolone propionate, epitiostanol, mepitiostane, and testolactone; abarelix; anastrozole; cetrorelix; deslorelin; exemestane; fadrozole; finasteride; formestane; histrelin (RL 0903); human chorionic gonadotropin; lanreotide; LDI 200 (Milkhaus); letrozole; leuprorelin; mifepristone; nafarelin; nafoxidine; osaterone; prednisone; thyrotropin alfa; and triptorelin.

In one embodiment, the additional anti-cancer agent (s) is an immunotherapeutic agent (i.e., immunotherapy). Non-limiting examples of immunotherapeutic agents include biological response modifiers, cytokine inhibitors, tumor vaccines, monoclonal antibodies, immune checkpoint inhibitors, colony-stimulating factors, and immunomodulators.

Non-limiting examples of biological response modifiers, including cytokine inhibitors (cytokines) such as interferons and interleukins, include interferon alfa/interferon alpha such as interferon alfa-2, interferon alfa-2a, interferon alfa-2b, interferon alfa-n1, interferon alfa-n3, interferon alfacon-1, peginterferon alfa-2a, peginterferon alfa-2b, and leukocyte alpha interferon; interferon beta such as interferon beta-1a, and interferon beta-1b; interferon gamma such as natural interferon gamma-1a, and interferon gamma-1b; aldesleukin; interleukin-1 beta; interleukin-2; oprelvekin; sonermin; tasonermin; and virulizin.

Non-limiting examples of tumor vaccines include APC 8015, AVICINE, bladder cancer vaccine, cancer vaccine (Biomira), gastrin 17 immunogen, Maruyama vaccine, melanoma lysate vaccine, melanoma oncolysate vaccine (New York Medical College), melanoma vaccine (New York University), melanoma vaccine (Sloan Kettering Institute), TICE® BCG (Bacillus Calmette-Guerin), and viral melanoma cell lysates vaccine (Royal Newcastle Hospital).

Non-limiting examples of monoclonal antibodies include abagovomab, adecatumumab, aflibercept, alemtuzumab, blinatumomab, brentuximab vedotin, CA 125 MAb (Biomira), cancer MAb (Japan Pharmaceutical Development), daclizumab, daratumumab, denosumab, edrecolomab, gemtuzumab zogamicin, HER-2 and Fc MAb (Medarex), ibritumomab tiuxetan, idiotypic 105AD7 MAb (CRC Technology), idiotypic CEA MAb (Trilex), ipilimumab, lintuzumab, LYM-1-iodine 131 MAb (Techni clone), mitumomab, moxetumomab, ofatumumab, polymorphic epithelial mucin-yttrium 90 MAb (Antisoma), ranibizumab, rituximab, and trastuzumab.

Non-limiting examples of immune checkpoint inhibitors include anti-PD-1 agents or antibodies such as cemiplimab, nivolumab, and pembrolizumab; anti-PD-L1 agents or antibodies such as atezolizumab, avelumab, and durvalumab; anti-CTLA-4 agents or antibodies such as ipilumumab; anti-LAG1 agents; and anti-OX40 agents.

Non-limiting examples of colony-stimulating factors include darbepoetin alfa, epoetin alfa, epoetin beta, filgrastim, granulocyte macrophage colony stimulating factor, lenograstim, leridistim, mirimostim, molgramostim, nartograstim, pegfilgrastim, and sargramostim.

Non-limiting examples of additional immunotherapeutic agents include BiTEs, CAR-T cells, GITR agonists, imiquimod, immunomodulatory imides (IMiDs), mismatched double stranded RNA (Ampligen), resiquimod, SRL 172, and thymalfasin.

In one embodiment, the additional anti-cancer agent(s) is a targeted therapy agent (i.e., targeted therapy). Targeted therapy agents include, for example, monoclonal antibodies and small molecule drugs. Non-limiting examples of targeted therapy agents include signal transduction inhibitors, growth factor inhibitors, tyrosine kinase inhibitors, EGFR inhibitors, histone deacetylase (HDAC) inhibitors, proteasome inhibitors, cell-cycle inhibitors, angiogenesis inhibitors, matrix-metalloproteinase (MMP) inhibitors, hepatocyte growth factor inhibitors, TOR inhibitors, KDR inhibitors, VEGF inhibitors, fibroblast growth factors (FGF) inhibitors, MEK inhibitors, ERK inhibitors, PI3K inhibitors, AKT inhibitors, MCL-1 inhibitors, BCL-2 inhibitors, SHP2 inhibitors, HER-2 inhibitors, BRAF-inhibitors, gene expression modulators, autophagy inhibitors, apoptosis inducers, antiproliferative agents, and glycolysis inhibitors.

Non-limiting examples of signal transduction inhibitors include tyrosine kinase inhibitors, multiple-kinase inhibitors, anlotinib, avapritinib, axitinib, dasatinib, dovitinib, imatinib, lenvatinib, lonidamine, nilotinib, nintedanib, pazopanib, pegvisomant, ponatinib, vandetanib, and EGFR inhibitory agents.

Non-limiting examples of EGFR inhibitory agents include small molecule antagonists of EGFR such as afatinib, brigatinib, erlotinib, gefitinib, lapatinib, and osimertinib; and antibody-based EGFR inhibitors, including any anti-EGFR antibody or antibody fragment that can partially or completely block EGFR activation by its natural ligand. Antibody-based EGFR inhibitory agents may include, for example, those described in Modjtahedi, H., et al., 1993, Br. J. Cancer 67:247-253; Teramoto, T., et al., 1996, Cancer 77:639-645; Goldstein et al, 1995, Clin. Cancer Res. 1: 1311-1318; Huang, S. M., et al., 1999, Cancer Res. 15:59(8): 1935-40; and Yang, X., et al., 1999, Cancer Res. 59: 1236-1243; monoclonal antibody Mab E7.6.3 (Yang, 1999 supra); Mab C225 (ATCC Accession No. HB-8508), or an antibody or antibody fragment having the binding specificity thereof; specific antisense nucleotide or siRNA; afatinib, cetuximab; matuzumab; necitumumab; nimotuzumab; panitumumab; and zalutumumab.

Non-limiting examples of histone deacetylase (HDAC) inhibitors include belinostat, panobinostat, romidepsin, and vorinostat.

Non-limiting examples of proteasome inhibitors include bortezomib, carfilzomib, ixazomib, marizomib (salinosporamide a), and oprozomib.

Non-limiting examples of cell-cycle inhibitors, including CDK inhibitors, include abemaciclib, alvocidib, palbociclib, and ribociclib.

In one embodiment, the additional anti-cancer agent(s) is an anti-angiogenic agent (or angiogenesis inhibitor) including, but not limited to, matrix-metalloproteinase (MMP) inhibitors; VEGF inhibitors; EGFR inhibitors; TOR inhibitors such as everolimus and temsirolimus; PDGFR kinase inhibitory agents such as crenolanib; HIF-1α inhibitors such as PX 478; HIF-2α inhibitors such as belzutifan and the HIF-2α inhibitors described in WO 2015/035223; fibroblast growth factor (FGF) or FGFR inhibitory agents such as B-FGF and RG 13577; hepatocyte growth factor inhibitors; KDR inhibitors; anti-Ang1 and anti-Ang2 agents; anti-Tie2 kinase inhibitory agents; Tek antagonists (US 2003/0162712; U.S. Pat. No. 6,413,932); anti-TWEAK agents (U.S. Pat. No. 6,727,225); ADAM distintegrin domain to antagonize the binding of integrin to its ligands (US 2002/0042368); anti-eph receptor and/or anti-ephrin antibodies or antigen binding regions (U.S. Pat. Nos. 5,981,245; 5,728,813; 5,969,110; 6,596,852; 6,232,447; and 6,057,124); and anti-PDGF-BB antagonists as well as antibodies or antigen binding regions specifically binding to PDGF-BB ligands.

Non-limiting examples of matrix-metalloproteinase (MMP) inhibitors include MMP-2 (matrix-metalloproteinase 2) inhibitors, MMP-9 (matrix-metalloproteinase 9) inhibitors, prinomastat, RO 32-3555, and RS 13-0830. Examples of useful matrix metalloproteinase inhibitors are described, for example, in WO 96/33172, WO 96/27583, EP 1004578, WO 98/07697, WO 98/03516, WO 98/34918, WO 98/34915, WO 98/33768, WO 98/30566, EP 0606046, EP 0931788, WO 90/05719, WO 99/52910, WO 99/52889, WO 99/29667, WO 1999/007675, EP 1786785, EP 1181017, US 2009/0012085, U.S. Pat. Nos. 5,863,949, 5,861,510, and EP 0780386. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e., MMP-1, MMP-3, MMP-4, MMP-5, MMP-6, MMP-7, MMP-8, MMP-10, MMP-11, MMP-12, and MMP-13).

Non-limiting examples of VEGF and VEGFR inhibitory agents include bevacizumab, cediranib, CEP 7055, CP 547632, KRN 633, orantinib, pazopanib, pegaptanib, pegaptanib octasodium, semaxanib, sorafenib, sunitinib, VEGF antagonist (Borean, Denmark), and VEGF-TRAP™

The other antitumor agent(s) may also be another anti-angiogenic agent including, but not limited to, 2-methoxyestradiol, AE 941, alemtuzumab, alpha-D148 Mab (Amgen, US), alphastatin, anecortave acetate, angiocidin, angiogenesis inhibitors, (SUGEN, US), angiostatin, anti-Vn Mab (Crucell, Netherlands), atiprimod, axitinib, AZD 9935, BAY RES 2690 (Bayer, Germany, BC 1 (Genoa Institute of Cancer Research, Italy), beloranib, benefin (Lane Labs, US), cabozantinib, CDP 791 (Celltech Group, UK), chondroitinase AC, cilengitide, combretastatin A4 prodrug, CP 564959 (OSI, US), CV247, CYC 381 (Harvard University, US), E 7820, EHT 0101, endostatin, enzastaurin hydrochloride, ER-68203-00 (IVAX, US), fibrinogen-E fragment, Flk-1 (ImClone Systems, US), forms of FLT 1 (VEGFR 1), FR-111142, GCS-100, GW 2286 (GlaxoSmithKline, UK), IL-8, ilomastat, IM-862, irsogladine, KM-2550 (Kyowa Hakko, Japan), lenalidomide, lenvatinib, MAb alpha5beta3 integrin, second generation (Applied Molecular Evolution, USA and Medlmmune, US), MAb VEGF (Xenova, UK), marimastat, maspin (Sosei, Japan), metastatin, motuporamine C, M-PGA, ombrabulin, OXI4503, PI 88, platelet factor 4, PPI 2458, ramucirumab, rBPI 21 and BPI-derived antiangiogenic (XOMA, US), regorafenib, SC-236, SD-7784 (Pfizer, US), SDX 103 (University of California at San Diego, US), SG 292 (Telios, US), SU-0879 (Pfizer, US), TAN-1120, TBC-1635, tesevatinib, tetrathiomolybdate, thalidomide, thrombospondin 1 inhibitor, Tie-2 ligands (Regeneron, US), tissue factor pathway inhibitors (EntreMed, US), tumor necrosis factor-alpha inhibitors, tumstatin, TZ 93, urokinase plasminogen activator inhibitors, vadimezan, vandetanib, vasostatin, vatalanib, VE-cadherin-2 antagonists, xanthorrhizol, XL 784 (Exelixis, US), ziv-aflibercept, and ZD 6126.

In embodiments, the other antitumor agent(s) is an additional active agent that disrupts or inhibits RAS-RAF-ERK or PI3K-AKT-TOR signaling pathways or is a PD-1 and/or PD-L1 antagonist. In embodiments, the other antitumor agent(s) is a RAF inhibitor, EGFR inhibitor, MEK inhibitor, ERK inhibitor, PI3K inhibitor, AKT inhibitor, TOR inhibitor, MCL-1 inhibitor, BCL-2 inhibitor, SHP2 inhibitor, proteasome inhibitor, or immune therapy, including monoclonal antibodies, immunomodulatory imides (IMiDs), anti-PD-1, anti-PDL-1, anti-CTLA4, anti-LAGl, and anti-OX40 agents, GITR agonists, CAR-T cells, and BiTEs.

Non-limiting examples of RAF inhibitors include dabrafenib, encorafenib, regorafenib, sorafenib, and vemurafenib.

Non-limiting examples of MEK inhibitors include binimetinib, CI-1040, cobimetinib, PD318088, PD325901, PD334581, PD98059, refametinib, selumetinib, and trametinib.

Non-limiting examples of ERK inhibitors include LY3214996, LTT462, MK-8353, SCH772984, ravoxertinib, ulixertinib, and an ERKi as described in WO 2017/068412.

Non-limiting examples of PI3K inhibitors include 17-hydroxywortmannin analogs (e.g., WO 06/044453); AEZS-136; alpelisib; AS-252424; buparlisib; CAL263; copanlisib; CUDC-907; dactolisib (WO 06/122806); demethoxyviridin; duvelisib; GNE-477; GSK1059615; IC87114; idelalisib; INK1117; LY294002; Palomid 529; paxalisib; perifosine; PI-103; PI-103 hydrochloride; pictilisib (e.g., WO 09/036,082; WO 09/055,730); PIK 90; PWT33597; SF1126; sonolisib; TGI 00-115; TGX-221; XL147; XL-765; wortmannin; and ZSTK474.

Non-limiting examples of AKT inhibitors include Akt-1-1 (inhibits Akt1) (Barnett et al. (2005) Biochem. J., 385 (Pt. 2), 399-408); Akt-1-1,2 (Barnett et al. (2005) Biochem. J. 385 (Pt. 2), 399-408); API-59CJ-Ome (e.g., Jin et al. (2004) Br. J. Cancer 91, 1808-12); 1-H-imidazo[4,5-c]pyridinyl compounds (e.g., WO05011700); indole-3-carbinol and derivatives thereof (e.g., U.S. Pat. No. 6,656,963; Sarkar and Li (2004) J Nutr. 134(12 Suppl), 3493S-3498S); perifosine, Dasmahapatra et al. (2004) Clin. Cancer Res. 10(15), 5242-52, 2004); phosphatidylinositol ether lipid analogues (e.g., Gills and Dennis (2004) Expert. Opin. Investig. Drugs 13, 787-97); triciribine (Yang et al. (2004) Cancer Res. 64, 4394-9); imidazooxazone compounds including trans-3-amino-1-methyl-3-[4-(3-phenyl-5H-imidazo[1,2-c]pyrido[3,4-e][1,3]oxazin-2-yl)phenyl]-cyclobutanol hydrochloride (WO 2012/137870); afuresertib; capivasertib; MK2206; and patasertib.

Non-limiting examples of TOR inhibitors include deforolimus; ATP-competitive TORC1/TORC2 inhibitors, including PI-103, PP242, PP30, and Torin 1; TOR inhibitors in FKBP12 enhancer, rapamycins and derivatives thereof, including temsirolimus, everolimus, WO 9409010; rapalogs, e.g. as disclosed in WO 98/02441 and WO 01/14387, e.g. AP23573, AP23464, or AP23841; 40-(2-hydroxyethyl)rapamycin, 40-[3-hydroxy(hydroxymethyl)methylpropanoate]-rapamycin; 40-epi-(tetrazolyl)-rapamycin (also called ABT578); 32-deoxorapamycin; 16-pentynyloxy-32 (S)-dihydrorapanycin, and other derivatives disclosed in WO 05/005434; derivatives disclosed in U.S. Pat. No. 5,258,389, WO 94/090101, WO 92/05179, U.S. Pat. Nos. 5,118,677, 5,118,678, 5,100,883, 5,151,413, 5,120,842, WO 93/111130, WO 94/02136, WO 94/02485, WO 95/14023, WO 94/02136, WO 95/16691, WO 96/41807, WO 96/41807 and U.S. Pat. No. 5,256,790; and phosphorus-containing rapamycin derivatives (e.g., WO 05/016252).

Non-limiting examples of MCL-1 inhibitors include AMG-176, MIK665, and S63845.

Non-limiting examples of SHP2 inhibitors include SHP2 inhibitors described in WO 2019/167000 and WO 2020/022323.

Additional non-limiting examples of additional anti-cancer agents that are suitable for combination use include 2-ethylhydrazide, 2,2′,2″-trichlorotriethylamine, ABVD, aceglatone, acemannan, aldophosphamide glycoside, alpharadin, amifostine, aminolevulinic acid, anagrelide, ANCER, ancestim, anti-CD22 immunotoxins, antitumorigenic herbs, apaziquone, arglabin, arsenic trioxide, azathioprine, BAM 002 (Novelos), bcl-2 (Genta), bestrabucil, biricodar, bisantrene, bromocriptine, brostallicin, bryostatin, buthionine sulfoximine, calyculin, cell-cycle nonspecific antineoplastic agents, celmoleukin, clodronate, clotrimazole, cytarabine ocfosfate, DA 3030 (Dong-A), defofamine, denileukin diftitox, dexrazoxane, diaziquone, dichloroacetic acid, dilazep, discodermolide, docosanol, doxercalciferol, edelfosine, eflornithine, EL532 (Elan), elfomithine, elsamitrucin, eniluracil, etanidazole, exisulind, ferruginol, folic acid replenisher such as frolinic acid, gacytosine, gallium nitrate, gimeracil/oteracil/tegafur combination (S-1), glycopine, histamine dihydrochloride, HIT diclofenac, HLA-B7 gene therapy (Vical), human fetal alpha fetoprotein, ibandronate, ibandronic acid, ICE chemotherapy regimen, imexon, iobenguane, IT-101 (CRLX101), laniquidar, LC 9018 (Yakult), leflunomide, lentinan, levamisole+fluorouracil, lovastatin, lucanthone, masoprocol, melarsoprol, metoclopramide, miltefosine, miproxifene, mitoguazone, mitozolomide, mopidamol, motexafin gadolinium, MX6 (Galderma), naloxone+pentazocine, nitracrine, nolatrexed, NSC 631570 octreotide (Ukrain), olaparib, P-30 protein, PAC-1, palifermin, pamidronate, pamidronic acid, pentosan polysulfate sodium, phenamet, picibanil, pixantrone, platinum, podophyllinic acid, porfimer sodium, PSK (Polysaccharide-K), rabbit antithymocyte polyclonal antibody, rasburiembodiment, retinoic acid, rhenium Re 186 etidronate, romurtide, samarium (153 Sm) lexidronam, sizofiran, sodium phenylacetate, sparfosic acid, spirogermanium, strontium-89 chloride, suramin, swainsonine, talaporfin, tariquidar, tazarotene, tegafur-uracil, temoporfin, tenuazonic acid, tetrachlorodecaoxide, thrombopoietin, tin ethyl etiopurpurin, tirapazamine, TLC ELL-12, tositumomab-iodine 131, trifluridine and tipiracil combination, troponin I (Harvard University, US), urethan, valspodar, verteporfin, zoledronic acid, and zosuquidar.

The present invention further provides a method for using the compound or pharmaceutically acceptable salt thereof of the present invention or pharmaceutical compositions provided herein, in combination with radiation therapy to treat cancer. Techniques for administering radiation therapy are known in the art, and these techniques can be used in the combination therapy described herein. The administration of the compound or pharmaceutically acceptable salt thereof of the present invention in this combination therapy can be determined as described herein.

Radiation therapy can be administered through one of several methods, or a combination of methods, including, without limitation, external-beam therapy, internal radiation therapy, implant radiation, stereotactic radiosurgery, systemic radiation therapy, radiotherapy and permanent or temporary interstitial brachy therapy. The term “brachytherapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide (s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive microspheres.

The present invention also provides methods for combination therapies in which the other antitumor agent is known to modulate other pathways, or other components of the same pathway, or even overlapping sets of target enzymes which are used in combination with a compound or pharmaceutically acceptable salt thereof of the present invention. In one embodiment, such therapy includes, but is not limited to, the combination of one or more compound or pharmaceutically acceptable salt thereof of the present invention with chemotherapeutic agents, immunotherapeutic agents, hormonal therapy agents, therapeutic antibodies, targeted therapy agents, and radiation treatment, to provide a synergistic or additive therapeutic effect.

In one embodiment, there is provided a method for modulating an activity of Ras protein including human KRAS G12D mutant protein, which comprises contacting the Ras protein with an effective amount of the compound of the present invention.

Examples of the activity to be modulated includes GTPase activity, nucleotide exchange, effector protein binding, effector protein activation, guanine exchange factor (GEF) binding, GEF-facilitated nucleotide exchange, phosphate release, nucleotide release, nucleotide binding, Ras, e.g., KRAS, localization in a cell, post-translational processing of Ras, e.g., KRAS, and posttranslation modification of Ras, e.g., KRAS, and preferably include KRAS localization in a cell, post-translational processing of KRAS, and posttranslation modification of KRAS. The “modulating” may be increasing or decreasing the activity of the Ras, e.g., KRAS protein.

In some embodiments, Ras, e.g., KRAS, protein exists in a living cell, such as a living cell which forms a part of a living object.

In the present specification, the “treatment” includes treatment carried out for the purpose of curing or ameliorating the disease, or for the purpose of suppressing the progression or recurrence of the disease or alleviating the symptoms.

The present invention also provides for the compound of the invention or pharmaceutically acceptable salt thereof, for use in therapy, or use of the compound of the invention or pharmaceutically acceptable salt thereof, in therapy. The present invention also provides for a pharmaceutical composition comprising the compound of the invention or pharmaceutically acceptable salt thereof, for use in the treatment of tumor, or use of the pharmaceutical composition comprising the compound of the invention or pharmaceutically acceptable salt thereof, for treating tumor. The present invention also provides for a pharmaceutical composition comprising the compound of the invention or pharmaceutically acceptable salt thereof, and an other antitumor agent, for use in the treatment of cancer, or use of the pharmaceutical composition comprising the compound of the invention or pharmaceutically acceptable salt thereof, and the other antitumor agent, for treating tumor.

### EXAMPLES

The invention will now be illustrated, but not limited, by reference to the specific embodiments described in the following examples. Compounds are named, for example, using an automated naming package such as AutoNom (MDL), using IUPAC rules or are as named by the chemical supplier. The reagents used in the Examples are commercially available products unless indicated otherwise.

Prepacked columns manufactured by Shoko Scientific Co., Ltd., or Biotage were used in silica gel column chromatography and basic silica gel column chromatography. An AL400 spectrometer (400 MHz; JEOL Ltd. (JEOL)), Mercury 400 (400 MHz; Varian), a Bruker Avance NEO spectrometer at 400 MHz or a Bruker Avance III Spectrometer at 500 MHz was used for NMR spectra.

For a deuterated solvent containing tetramethylsilane, tetramethylsilane was used as the internal reference. For other cases, measurement was performed using an NMR solvent as the internal reference. All 5 values are indicated in ppm. Microwave reaction was performed using an Initiator (trademark) manufactured by Biotage. Reverse phase preparative HPLC column chromatography was performed at the following conditions.
Column: CAPCELL PAK C18 AQ manufactured by SHISEIDO, 30×50 mm, 5 μm
UV detection: 254 nm
Column flow rate: 40 mL/min
Mobile phase: water/acetonitrile (0.1% formic acid)
Injection volume: 1.0 mL
Basic gradient method: water/acetonitrile 0%-50% (8 minutes)
Column: XSelect CSH C18 OBD manufactured by Waters. 130A. 5 μm. 19×100 mm
UV detection: 254 nm
Column flow rate: 18 mL/min
Mobile phase: water/acetonitrile (0.1% formic acid)
Injection volume: 1.0 mL
Basic gradient method: water/acetonitrile 15%-40% (8 minutes)

In the examples, the following abbreviations are used.
Ac acetyl
aq. aqueous
Boc tert-butyloxycarbonyl
BINAP (2,2′-bis(diphenylphosphino)-1,1′-binaphthyl)
Cbz carboxybenzyl 2-(Dicyclohexylphosphino)-2′-(dimethylamino)biphenyl
dba dibenzylideneacetone
DCM dichloromethane
DIPEA N,N-diisopropylethylamine
DMA N,N-dimethylacetamide
DMF N,N-dimethylformamide
DMSO dimethyl sulfoxide
dppf 1,1′-Bis(diphenylphosphino)ferrocene
EtOAc ethyl acetate
EtOH ethanol
h hour
HATU O-(7-azabenzotriazol-1-yl)-N,N,N′,N′-tetramethyluronium hexafluorophosphate
HPLC high performance liquid chromatography
LHMDS Lithium hexamethyldisilazide
MeCN acetonitrile
MeOH methanol
min. minutes
MS mass spectrometry
NMR nuclear magnetic resonance spectroscopy
RT room temperature
RUPHOS Dicyclohexyl(2′,6′-diisopropoxy-[1,1′-biphenyl]-2-yl)phosphine
Sat. saturated
TBAF tetrabutylammonium fluoride
TEA triethylamine
TFA trifluoroacetic acid
THF tetrahydrofuran
TLC thin Layer chromatography
Xantphos 4,5-Bis (diphenylphosphino)-9,9-dimethylxanthene
Z-chloride benzyl chloroformate

### Preparation 1: tert-butyl 3-(2-((1-((dimethylamino)methyl)cyclopropyl)methoxy)-5,6,7,8-tetrahydropyrido[3,4-d]pyrimidin-4-yl)-3,8-diazabicyclo[3.2.1]octane-8-carboxylate in Scheme 1

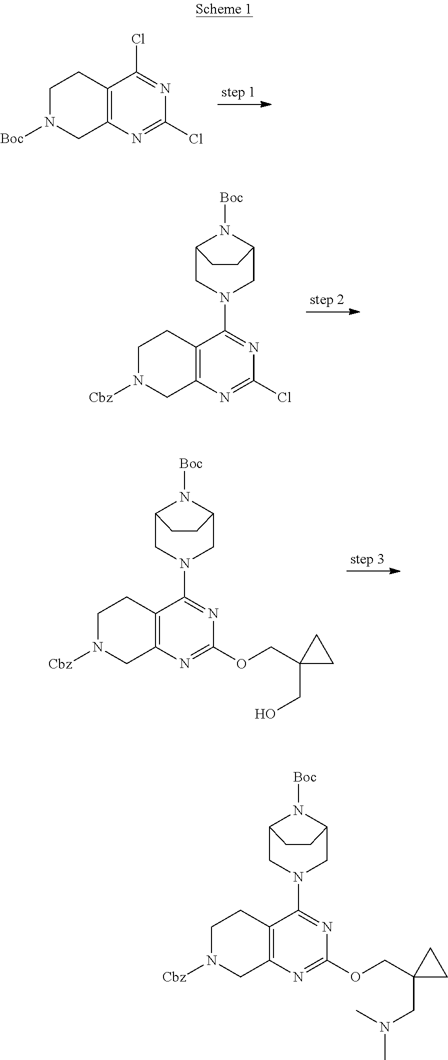 [SMILES: CC(C)(C)OC(=O)N1CCc2c(Cl)nc(Cl)nc2C1 ; CC(C)(C)OC(=O)N1C2CCC1CN(c1nc(Cl)nc3c1CCN(C(=O)OCc1ccccc1)C3)C2 ; CC(C)(C)OC(=O)N1C2CCC1CN(c1nc(OCC3(CO)CC3)nc3c1CCN(C(=O)OCc1ccccc1)C3)C2 ; CN(C)CC1(COc2nc3c(c(N4CC5CCC(C4)N5C(=O)OC(C)(C)C)n2)CCN(C(=O)OCc2ccccc2)C3)CC1]

### Step 1: Benzyl 4-(8-(tert-butoxycarbonyl)-3,8-diazabicyclo[3.2.1]octan-3-yl)-2-chloro-5,8-dihydropyrido[3,4-d]pyrimidine-7(6H)-carboxylate

To a solution of tert-butyl 2,4-dichloro-5,8-dihydropyrido[3,4-d]pyrimidine-7(6H)-carboxylate (20.0 g, 65.7 mmol) in CHCl3 (200 mL) was added TFA (200 mL) at room temperature and stirred for 1 h. The mixture was concentrated to give the corresponding amine, which was used without further purification. To a solution of the amine in CH2Cl2 (400 mL) was added iPr2NEt (40 mL), benzyl chloroformate (15.9 mL), and DMAP (803 mg) at 0 C.

After stirring at room temperature for 1 h, CH2Cl2 was evaporated and the mixture was diluted with EtOAc. The organic layer was washed with H2O and brine, dried over Na2SO4 and evaporated to give the corresponding product, which was used without further purification.

To a solution of the product in DMA (660 mL) was added tert-butyl 3,8-diazabicyclo[3.2.1]octane-8-carboxylate (34.9 g) and iPr2Net (28.6 mL) at room temperature. After stirring at room temperature for 30 min, the reaction mixture was diluted with EtOAc and H2O. The organic layer was separated, and the aqueous layer was extracted with EtOAc.

The combined organic layer was washed with H2O and brine, dried over Na2SO4, and evaporated. The resulting residue was purified by silica gel column chromatography to give benzyl 4-(-8-(tert-butoxycarbonyl)-3,8-diazabicyclo[3.2.1]octan-3-yl)-2-chloro-5,8-dihydropyrido[3,4-d]pyrimidine-7(6H)-carboxylate (76.0 g).

1H NMR (400 MHz, CHLOROFORM-d) δ=7.46-7.30 (m, 5H), 5.20 (s, 2H), 4.61 (s, 2H), 4.46-4.19 (m, 2H), 3.89 (d, J=12.0 Hz, 2H), 3.79-3.43 (m, 2H), 3.28 (d, J=11.0 Hz, 2H), 2.80-2.54 (m, 2H), 2.03-1.89 (m, 2H), 1.86-1.72 (m, 2H), 1.51 (s, 9H) ESI-MS m/z 514, 516(MH+)

### Step 2: Benzyl 4-(8-(tert-butoxycarbonyl)-3,8-diazabicyclo[3.2.1]octan-3-yl)-2-((1-(hydroxymethyl)cyclopropyl)methoxy)-5,8-dihydropyrido[3,4-d]pyrimidine-7(6H)-carboxylate

To a solution of benzyl 4-(8-(tert-butoxycarbonyl)-3,8-diazabicyclo[3.2.1]octan-3-yl)-2-chloro-5,8-dihydropyrido[3,4-d]pyrimidine-7(6H)-carboxylate (30.1 g, 58.5 mmol), cyclopropane-1,1-diyldimethanol (12.0 g), and Cs2CO3 (47.7 g) in 1,4-dioxane (600 mL) was added Ruphos Pd G3 (4.89 g) at room temperature. After stirring at 100 C for 3 h, the reaction mixture was cooled to rt and diluted with EtOAc and H2O.

The mixture was filtrated through Celite and separated. The aqueous layer was extracted with EtOAc, and the combined organic layer was washed with brine, dried over Na2SO4 and evaporated. The resulting residue was purified by silica gel column chromatography to give benzyl 4-(8-(tert-butoxycarbonyl)-3,8-diazabicyclo[3.2.1]octan-3-yl)-2-((1-(hydroxymethyl)cyclopropyl)methoxy)-5,8-dihydropyrido[3,4-d]pyrimidine-7(6H)-carboxylate (20.4 g).

1H NMR (400 MHz, CHLOROFORM-d) δ=7.51-7.31 (m, 5H), 5.20 (s, 2H), 4.65-4.48 (m, 2H), 4.42-4.18 (m, 4H), 3.91-3.42 (m, 6H), 3.37-2.98 (m, 3H), 2.77-2.54 (m, J=4.6 Hz, 2H), 2.02-1.76 (m, 4H), 1.50 (s, 9H), 0.69-0.62 (m, 2H), 0.62-0.56 (m, 2H) ESI-MS m/z 580 (MH+)

### Step 3: tert-butyl 3-(2-((1-((dimethylamino)methyl)cyclopropyl)methoxy)-5,6,7,8-tetrahydropyrido[3,4-d]pyrimidin-4-yl)-3,8-diazabicyclo[3.2.1]octane-8-carboxylate

To a solution of benzyl 4-(8-(tert-butoxycarbonyl)-3,8-diazabicyclo[3.2.1]octan-3-yl)-2-((1-(hydroxymethyl)cyclopropyl)methoxy)-5,8-dihydropyrido[3,4-d]pyrimidine-7(6H)-carboxylate (2.5 g) in EtOAc (25 mL) was added Et3N (1.8 mL) and MsCl (0.50 m) at 0 C. After stirring at rt for 30 min, the reaction mixture was filtrated through Celite and washed with EtOAc.

The filtrate was washed with sat.NaHCO₃ and brine, dried over Na2SO4 and evaporated to give the Ms adduct, which was used without further purification. To a solution of the Ms adduct and K2CO3 (2.98 g) in DMA (25 mL) was added 2 M Me2NH in THF (21.6 mL). After stirring at 45 C for 3 h, the reaction mixture was diluted with EtOAc. The combined organic layer was washed with H2O and brine, dried over Na2SO4 and evaporated.

The resulting residue was purified by silica gel column chromatography to give the dimethyl product, which was used without further purification. To a solution of the dimethyl product in EtOH (50 mL) was added Pd(OH)2 on carbon (1.25 g). After replacing under H2 atmosphere and stirring at rt for 4 h, the reaction mixture was filtrated through Celite and washed with EtOH and the filtrate was evaporated. The resulting residue was purified by silica gel column chromatography to give tert-butyl 3-(2-((1-((dimethylamino)methyl)cyclopropyl)methoxy)-5,6,7,8-tetrahydropyrido[3,4-d]pyrimidin-4-yl)-3,8-diazabicyclo[3.2.1]octane-8-carboxylate (1.33 g).

1H NMR (400 MHz, CHLOROFORM-d) δ=4.42-4.20 (m, 2H), 4.15 (s, 2H), 3.94 (s, 2H), 3.79 (d, J=12.3 Hz, 2H), 3.31-3.08 (m, 2H), 3.05-2.95 (m, 2H), 2.61-2.52 (m, 2H), 2.34 (s, 2H), 2.26 (s, 6H), 2.01-1.80 (m, 4H), 1.51 (s, 9H), 0.69-0.57 (m, 2H), 0.49-0.37 (m, 2H) ESI-MS m/z 473 (MH+)

The below synthetic intermediates were prepared using similar chemistry in Scheme 1 and procedure used to prepare tert-butyl 3-(2-((1-((dimethylamino)methyl)cyclopropyl)methoxy)-5,6,7,8-tetrahydropyrido[3,4-d]pyrimidin-4-yl)-3,8-diazabicyclo[3.2.1]octane-8-carboxylate

### Preparation 2: tert-butyl 3-(2-((1-(((R)-3-fluoropyrrolidin-1-yl)methyl)cyclopropyl)methoxy)-5,6,7,8-tetrahydropyrido[3,4-d]pyrimidin-4-yl)-3,8-diazabicyclo[3.2.1]octane-8-carboxylate

The title compound was obtained in accordance with Preparation 1, with the exception that (R)-3-fluoropyrrolidine was used instead of 2 M Me2NH in THF.1H NMR (400 MHz, CHLOROFORM-d) δ=5.31-5.01 (m, 1H), 4.42-4.08 (m, 4H), 3.94 (s, 2H), 3.80 (d, J=12.3 Hz, 2H), 3.20 (s, 2H), 3.07-2.96 (m, 2H), 2.94-2.69 (m, 3H), 2.67-2.40 (m, 5H), 2.23-1.79 (m, 6H), 1.51 (s, 9H), 0.67-0.58 (m, 2H), 0.49-0.41 (m, 2H) ESI-MS m/z 517 (MH+)

### Preparation 3: tert-butyl 3-(2-((1-(morpholinomethyl)cyclopropyl)methoxy)-5,6,7,8-tetrahydropyrido[3,4-d]pyrimidin-4-yl)-3,8-diazabicyclo[3.2.1]octane-8-carboxylate

The title compound was obtained in accordance with Preparation 1, with the exception that morpholine was used instead of 2 M Me2NH in THF.

1H NMR (400 MHz, CHLOROFORM-d) δ=4.43-4.13 (m, 4H), 3.87-3.73 (m, 2H), 3.73-3.62 (m, 4H), 3.32-3.10 (m, 2H), 3.07-2.94 (m, 2H), 2.80-2.36 (m, 8H), 2.02-1.72 (m, 6H), 1.50 (s, 9H), 0.68-0.60 (m, 2H), 0.44-0.39 (m, 2H) MASS ESI-MS m/z 515 (MH+)

### Preparation 4: 1,8-dibromo-3-(methoxymethoxy)naphthalene in Scheme 2

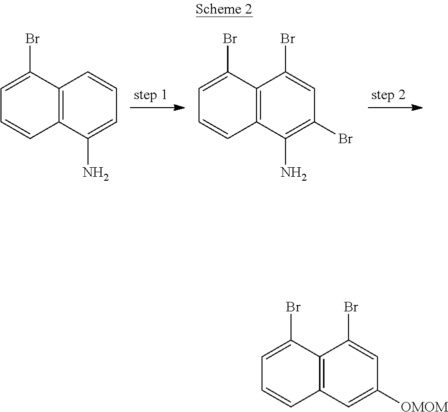

### Step 1: 2,4,5-tribromonaphthalen-1-amine

To a solution of 5-bromonaphthalen-1-amine (63 g, 280 mmol) in DMA (1260 mL) was added NBS (106 g) at 0° C. The mixture was allowed to warm to room temperature and stirred for 3 h. The reaction mixture was diluted with Na2SO3 (75 g) in H2O (380 mL) and NaHCO₃(24 g) in H2O (1100 mL) and stirred for 1 h. The precipitate was collected by filtration and washed with water to give 2,4,5-tribromonaphthalen-1-amine (97 g) as a purple solid.

1H NMR (400 MHz, CDCl3) δ 7.99 (s, 1H), 7.97-7.95 (1H, m), 7.86-7.84 (1H, m), 7.31-7.29 (1H, m), 4.65 (2H, brs). LCMS (ESI): 379(M+H)

### Step 2: 1,8-dibromo-3-(methoxymethoxy)naphthalene

To a suspension of 2,4,5-tribromonaphthalen-1-amine (60 g, 160 mmol) in AcOH (780 mL) and propionic acid (300 mL) was added NaNO2 (11 g) portionwise at 0° C. and the reaction mixture was stirred for 20 min. The reaction mixture was diluted with H2O (1800 mL) at 0° C. and stirred for 1 h. The slurry was filtrated and the solid was washed with H2O to give 4,5-dibromo-2-hydroxynaphthalene-1-diazonium, which was used without further purification. To a suspension of 4,5-dibromo-2-hydroxynaphthalene-1-diazonium in EtOH (1600 mL) was added NaBH4 (15 g) portionwise at 0° C. and the reaction mixture was stirred for 30 min.

The mixture was allowed to warm to room temperature and stirred overnight. The reaction mixture was cooled to 0 C and diluted with water (1500 mL) and 5M HCl aq. (79 mL). The mixture was evaporated to remove EtOH and extracted with CHCl3. The combined organic layer was washed with brine and dried over Na2SO4 and evaporated in vacuo to give 4,5-dibromonaphthalen-2-ol, which was used without further purification.

To a solution of 4,5-dibromonaphthalen-2-ol in CH2Cl2 (900 mL) was added iPr2NEt (170 mL) and MOMCl (36 m) at 0 C. After stirring at room temperature for 1 h, the reaction mixture was diluted with EtOAc and sat.NaHCO₃. The organic layer was separated, and the aqueous layer was extracted with EtOAc.

The combined organic layer was washed with brine, dried over Na2SO4, and evaporated. The resulting residue was purified by silica gel column chromatography to give 1,8-dibromo-3-(methoxymethoxy)naphthalene (17.4 g).

1H NMR (400 MHz, CHLOROFORM-d) δ=7.82 (dd, J=1.3, 7.4 Hz, 1H), 7.75-7.71 (m, 2H), 7.41 (d, J=2.6 Hz, 1H), 7.26-7.20 (m, J=8.0, 8.0 Hz, 1H), 5.29 (s, 2H), 3.53 (s, 3H).

The below synthetic intermediates were prepared using similar chemistry in Scheme 2 and procedure used to prepare 1,8-dibromo-3-(methoxymethoxy)naphthalene.

### Preparation 5: 1-bromo-3-(methoxymethoxy)-8-methylnaphthalene

The title compound was obtained in accordance with Preparation 4, with the exception that 5-methylnaphthalen-1-amine was used instead of 5-bromonaphthalen-1-amine.

1H NMR (500 MHz, CHLOROFORM-d) δ=7.61-7.59 (m, 2H), 7.34 (d, J=2.6 Hz, 1H), 7.28 (t, J=7.4 Hz, 1H), 7.21 (td, J=1.2, 6.9 Hz, 1H), 5.26 (s, 2H), 3.51 (s, 3H), 3.08 (s, 3H)

### Preparation 6: Benzyl 4-(8-(tert-butoxycarbonyl)-3,8-diazabicyclo[3.2.1]octan-3-yl)-2-chloro-5,7-dihydro-6H-pyrrolo[3,4-d]pyrimidine-6-carboxylate

To a solution of tert-butyl 2,4-dichloro-5,7-dihydro-6H-pyrrolo[3,4-d]pyrimidine-6-carboxylate (20.0 g, 68.9 mmol) in CHCl₃ (100 mL) was added TFA (100 mL) at room temperature and stirred for 1 h under nitrogen atmosphere. The mixture was concentrated to give the corresponding amine, which was used without further purification. To a solution of the amine in CH₂Cl₂ (200 mL) was added iPr₂NEt (42 mL), benzyl chloroformate (16.7 mL), and DMAP (842 mg) at 0° C. under nitrogen atmosphere.

After stirring at room temperature for 1 h, CH₂Cl₂ was evaporated and the mixture was diluted with EtOAc. The organic layer was washed with H₂O and brine, dried over MgSO₄ and evaporated to give the corresponding product, which was used without further purification. To a solution of the product in DMA (400 mL) was added tert-butyl 3,8-diazabicyclo[3.2.1]octane-8-carboxylate (14.6 g) and iPr₂NEt (12.0 mL) at room temperature under nitrogen atmosphere.

After stirring at room temperature for 1 h, the reaction mixture was diluted with EtOAc and H₂O. After phase separation, the organic layer was separated, and the organic layer was washed with brine, dried over MgSO₄, and evaporated. The resulting residue was purified by silica gel column chromatography to give benzyl 4-(8-(tert-butoxycarbonyl)-3,8-diazabicyclo[3.2.1]octan-3-yl)-2-chloro-5,7-dihydro-6H-pyrrolo[3,4-d]pyrimidine-6-carboxylate (22.2 g).

ESI-MS m/z 500, 502 (MH+)

### Preparation 7: Benzyl 4-(8-(tert-butoxycarbonyl)-3,8-diazabicyclo[3.2.1]octan-3-yl)-2-((1-(hydroxymethyl) cyclopropyl)methoxy)-5,7-dihydro-6H-pyrrolo[3,4-d]pyrimidine-6-carboxylate

To a solution of benzyl 4-(8-(tert-butoxycarbonyl)-3,8-diazabicyclo[3.2.1]octan-3-yl)-2-chloro-5,7-dihydro-6H-pyrrolo[3,4-d]pyrimidine-6-carboxylate (22.2 g, 44.5 mmol), cyclopropane-1,1-diyldimethanol (13.6 g), and Cs₂CO₃ (43.5 g) in 1,4-dioxane (445 mL) was added Ruphos Pd G3 (3.72 g) at room temperature.

After stirring at 100° C. for 3 h, the reaction mixture was cooled to room temperature and diluted with EtOAc and H₂O. After phase separation, the organic layer was dried over MgSO₄ and evaporated. The resulting residue was purified by silica gel column chromatography to give benzyl 4-(8-(tert-butoxycarbonyl)-3,8-diazabicyclo[3.2.1]octan-3-yl)-2-((1-(hydroxymethyl)cyclopropyl)methoxy)-5,7-dihydro-6H-pyrrolo[3,4-d]pyrimidine-6-carboxylate (14.4 g).

ESI-MS m/z 566 (MH+)

### Preparation 8: tert-butyl 3-(2-((1-(morpholinomethyl) cyclopropyl) methoxy)-6,7-dihydro-5H-pyrrolo[3,4-d]pyrimidin-4-yl)-3,8-diazabicyclo[3.2.1]octane-8-carboxylate

### Step 1: Benzyl 4-(8-(tert-butoxycarbonyl)-3,8-diazabicyclo[3.2.1]octan-3-yl)-2-((1-(morpholinomethyl) cyclopropyl)methoxy)-5,7-dihydro-6H-pyrrolo[3,4-d]pyrimidine-6-carboxylate

To a solution of benzyl 4-(8-(tert-butoxycarbonyl)-3,8-diazabicyclo[3.2.1]octan-3-yl)-2-((1-(hydroxymethyl) cyclopropyl)methoxy)-5,7-dihydro-6H-pyrrolo[3,4-d]pyrimidine-6-carboxylate (1.00 g, 1.77 mmol), DIPEA (0.92 mL, 5.30 mmol) in DMF (10 mL) was added methanesulfonyl chloride (0.275 mL, 3.54 mmol) at 0° C., the mixture was stirred at the same temperature for 30 min.

To the mixture was added morpholine (3.1 mL, 35.4 mmol) and potassium carbonate (2.00 g, 14.1 mmol) at rt and stirred at 50 degree for another 2 d. The mixture was cooled to rt, diluted with EtOAc and water, extracted with EtOAc. The organic phase was washed with brine, dried over Na₂SO₄, filtered, concentrated in vacuo. The residue was purified by column chromatography on silica gel to give a title compound (1.15 g, 1.81 mmol, quant.). ESI-MS: [M+H]⁺=635.

### Step 2: tert-butyl 3-(2-((1-(morpholinomethyl)cyclopropyl)methoxy)-6,7-dihydro-5H-pyrrolo[3,4-d]pyrimidin-4-yl)-3,8-diazabicyclo[3.2.1]octane-8-carboxylate

To a solution of benzyl 4-(8-(tert-butoxycarbonyl)-3,8-diazabicyclo[3.2.1]octan-3-yl)-2-((1-(morpholinomethyl)cyclopropyl)methoxy)-5,7-dihydro-6H-pyrrolo[3,4-d]pyrimidine-6-carboxylate (1.15 g, 1.81 mmol) in ethanol (10 mL) was added palladium hydroxide on carbon (690 mg), and stirred at rt overnight.

The mixture was filtered through a pad of Celite, and washed with ethanol. The filtrate was concentrated and the residue was purified by column chromatography on NH-silica gel to give a title compound (705 mg, 1.41 mmol, 78%). ESI-MS: [M+H]⁺=501.

### Preparation 9: tert-butyl 3-(2-((1-(((R)-3-fluoropyrrolidin-1-yl)methyl)cyclopropyl)methoxy)-6,7-dihydro-5H-pyrrolo[3,4-d]pyrimidin-4-yl)-3,8-diazabicyclo[3.2.1]octane-8-carboxylate

### Step 1: Benzyl 4-(8-(tert-butoxycarbonyl)-3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidine-6-carboxylate

Prepared from benzyl 4-(8-(tert-butoxycarbonyl)-3,8-diazabicyclo[3.2.1]octan-3-yl)-2-((1-(hydroxymethyl)cyclopropyl)methoxy)-5,7-dihydro-6H-pyrrolo[3,4-d]pyrimidine-6-carboxylate (1.00 g, 1.77 mmol) and (R)-3-fluoropyrrolidine hydrochloride (4.44 g, 35.4 mmol) using same procedure as step 1 of preparation 8, to give a title compound (1.09 g, 1.71 mmol, 97%). ESI-MS: [M+H]⁺=637.

Step 2: tert-butyl-3-(2-((1-(((R)-3-fluoropyrrolidin-1-yl) methyl) cyclopropyl) methoxy)-6,7-dihydro-5H-pyrrolo[3,4-d]pyrimidin-4-yl)-3,8-diazabicyclo[3.2.1]octane-8-carboxylate

Prepared from benzyl 4-(8-(tert-butoxycarbonyl)-3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidine-6-carboxylate (1.09 g, 1.71 mmol) using same method as step 2 of preparation 8 to give a title compound (562 mg, 1.12 mmol, 63%). ESI-MS: [M+H]⁺=503.

### Preparation 10: 3-hydroxy-8-iodo-naphthalene-1-carboxylic acid

### Step 1: 3-amino-8-iodo-naphthalene-1-carboxylic acid

To a mixture of 8-iodo-3-nitro-naphthalene-1-carboxylic acid¹) (1 g, 2.9 mmol) in Ethyl acetate (40 mL)-Ethanol(15 mL) was added 5% Rh/C (0.5 g) and the flask charged with H₂. The mixture was stirred at room temperature. After 24 h, the reaction mixture was filtered through a pad of celite and the filtrate concentrated to dryness to afford crude 3-amino-8-iodo-naphthalene-1-carboxylic acid (0.94 g) as a brown solid which was used in the next step without purification.

1H NMR (400 MHz, DMSO-d₆) δ=5.71 (brs, 2H), 6.87 (d, J=2.4 Hz, 1H), 7.00 (dd, J=8.1, 7.3 Hz, 1H), 7.12 (d, J=2.4 Hz, 1H), 7.60 (d, J=8.2 Hz, 1H), 7.8 (d, J=7.3 Hz, 1H), 13.14 (brs, 1H).

LCMS (ESI): 314 (M+H)

1) Yakugaku Zasshi, 98 (3), 358-65; 1978.

### Step 2: 3-hydroxy-8-iodo-naphthalene-1-carboxylic acid

To an ice cold mixture of crude 3-amino-8-iodo-naphthalene-1-carboxylic acid (0.94 g) in 1M aqueous sulfuric acid (38 mL) was slowly added dropwise with a solution of sodium nitrite (0.228 g, 3.3 mmol) in water (1 mL). The mixture was stirred for 1 hour and warmed to room temperature. The reaction mixture was added dropwise to a refluxing solution of 40% aqueous sulfuric acid (108 mL).

The reaction mixture was heated under reflux for 1 hour and then quickly hot-filtered through a plug of glass wool to remove insoluble, charred material. The filtrate was cooled to room temperature, and a precipitate was formed. The precipitate was collected by filtration and washed with water to give 3-hydroxy-8-iodo-naphthalene-1-carboxylic acid (0.54 g).

¹H NMR (400 MHz, DMSO-d₆) δ=7.13 (dd, J=8.1, 7.3 Hz, 1H), 7.22 (d, J=2.8 Hz, 1H), 7.24 (d, J=2.8 Hz, 1H), 7.82 (dd, J=8.3, 0.8 Hz, 1H), 8.01 (dd, J=7.3, 1.2 Hz, 1H), 10.22 (brs, 1H).

LCMS (ESI): 315 (M+H)

### Preparation 11: 4-(4,4,5,5-tetramethyl-1,3,2-dioxaborolan-2-yl) naphthalen-2-ol

To a solution of 4-bromonaphthalen-2-ol (4.0 g, 17.9 mmol) in 1,2-dimethoxyethane (56 mL) were added 4,4,5,5-tetramethyl-2-(4,4,5,5-tetramethyl-1,3,2-dioxaborolan-2-yl)-1,3,2-dioxaborolane (5.60 g, 22 mmol), [1,1′-Bis (diphenylphosphino)ferrocene]dichloropalladium(II), complex with dichloromethane (0.392 g, 0.480 mmol) and potassium acetate (5.68 g, 57.9 mmol) at room temperature.

The mixture was heated at 120° C. for 45 min. Water was added and the mixture was extracted with EtOAc. The organic layer was washed with water and brine, dried over anhydrous Na₂SO₄, filtered, and concentrated in vacuo. The residue was purified by column chromatography on silica gel (gradient elution, 00-50% EtOAc/hexane) to give the title compound (3.29 g).

ESI-MS: [M+H]⁺=271.

### Preparation 12: 2-[3-(methoxymethoxy)-1-naphthyl]-4,4,5,5-tetramethyl-1,3,2-dioxaborolane

### (Step-1) Synthesis of 1-bromo-3-(methoxymethoxy)naphthalene

To a solution of 4-bromonaphthalen-2-ol (6.00 g, 26.9 mmol) in dichloromethane (135 mL) were added N,N-diisopropylethylamine (9.37 mL, 53.8 mmol) at room temperature. To the mixture were added chloro(methoxy)methane (2.43 mL, 32.3 mmol) at 0° C. by dropwise. The mixture was stirred at room temperature for 15h. saturated NaHCO₃ in water was added and the mixture was extracted with CHCl₃.

The organic layer was washed with water and brine, dried over anhydrous Na₂SO₄, filtered, and concentrated in vacuo. The residue was purified by column chromatography on silica gel (gradient elution, 00-50% EtOAc/hexane) to give the title compound (7.00 g, 26.2 mmol, 97%).

### (Step-2) Synthesis of 2-[3-(methoxymethoxy)-1-naphthyl]-4,4,5,5-tetramethyl-1,3,2-dioxaborolane

Prepared from 1-bromo-3-(methoxymethoxy)naphthalene (7.00 g, 26.2 mmol) using same procedure as preparation 11, to give the title compound (7.63 g, 24.3 mmol, 93%).

### Preparation 13: tert-butyl (1S,4S)-5-(2-((1-((dimethylamino)methyl)cyclopropyl)methoxy)-5,6,7,8-tetrahydropyrido[3,4-d]pyrimidin-4-yl)-2,5-diazabicyclo[2.2.2]octane-2-carboxylate

The title compound was obtained in accordance with Preparation 1, with the exception that tert-butyl (1S,4S)-2,5-diazabicyclo[2.2.2]octane-2-carboxylate was used instead of tert-butyl 3,8-diazabicyclo[3.2.1]octane-8-carboxylate.

ESI-MS m/z 473 (MH+)

### Preparation 14: tert-butyl 3-(2-((1-((dimethylamino)methyl)-2,2-difluorocyclopropyl)methoxy)-5,6,7,8-tetrahydropyrido[3,4-d]pyrimidin-4-yl)-3,8-diazabicyclo[3.2.1]octane-8-carboxylate

The title compound was obtained in accordance with Preparation 1, with the exception that (2,2-difluorocyclopropane-1,1-diyl)dimethanol was used instead of cyclopropane-1,1-diyldimethanol

ESI-MS m/z 509 (MH+)

### Preparation 15: tert-butyl (1S,4S)-5-(2-((1-(morpholinomethyl)cyclopropyl)methoxy)-5,6,7,8-tetrahydropyrido[3,4-d]pyrimidin-4-yl)-2,5-diazabicyclo[2.2.2]octane-2-carboxylate

The title compound was obtained in accordance with Preparation 1, with the exception that tert-butyl (1S,4S)-2,5-diazabicyclo[2.2.2]octane-2-carboxylate and morpholine was used instead of tert-butyl 3,8-diazabicyclo[3.2.1]octane-8-carboxylate and 2 M Me2NH in THF ESI-MS m/z 515 (MH+)

### Preparation 16: tert-butyl 3-(2-((1-((dimethylamino)methyl)-2,2-dimethylcyclopropyl)methoxy)-5,6,7,8-tetrahydropyrido[3,4-d]pyrimidin-4-yl)-3,8-diazabicyclo[3.2.1]octane-8-carboxylate

The title compound was obtained in accordance with Preparation 1, with the exception that (2,2-dimethylcyclopropane-1,1-diyl)dimethanol was used instead of cyclopropane-1,1-diyldimethanol ESI-MS m/z 501 (MH+)

### Preparation 17: tert-butyl (1,8-dibromoisoquinolin-3-yl)carbamate In Scheme 3

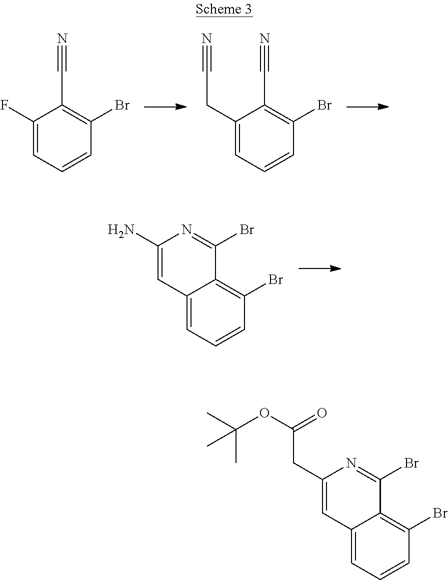 [SMILES: N#Cc1c(F)cccc1Br ; N#CCc1cccc(Br)c1C#N ; Nc1cc2cccc(Br)c2c(Br)n1 ; CC(C)(C)OC(=O)Cc1cc2cccc(Br)c2c(Br)n1]

### Step 1: 2-bromo-6-(cyanomethyl)benzonitrile

To a mixture of Sodium hydride (50 wt % 2.0 g) in DMSO (15 mL) was added methyl 2-cyanoacetate (4.4 mL) at 0 degree. The mixture was stirred for 30 min at room temperature. The reaction mixture was added the solution of 2-bromo-6-fluoro-benzonitrile (5.0 g) in DMSO (25 mL) dropwise over 30 min and stirred at 90 degree C. for 2h. The mixture was added water (90 mL). After stirring at 100 degree C. overnight, the reaction mixture was cooled to 0 degree C. and quenched with 0.1N HCl aq (50 mL). After stirring at same temperature for 2h, the precipitate was collected by filtration and washed with water to give 2-bromo-6-(cyanomethyl)benzonitrile (5.41 g) as a white green powder.

1H NMR (400 MHz, CHLOROFORM-d) δ=7.76-7.70 (m, 1H), 7.68-7.64 (m, 1H), 7.59-7.53 (m, 1H), 4.07-4.04 (m, 2H), 1.28 (s, 2H), 0.93-0.83 (m, 1H). LCMS (ESI): 220, 222 (M+H)

### Step 2: 1,8-dibromoisoquinolin-3-amine

To a mixture of 2-bromo-6-(cyanomethyl)benzonitrile (1.0 g) in dichloroacetic acid (5 mL) was added hydrobromic acid (30% in AcOH, 4.5 mL) at room temperature. After stirring for 15 min, the reaction mixture was quenched with sat. K₂CO₃ aq. The precipitate was collected by filtration and washed with water to give 1,8-dibromoisoquinolin-3-amine (0.94 g) as a yellow solid.

1H NMR (400 MHz, DMSO-d6) δ=7.65-7.57 (m, 2H), 7.30-7.24 (m, 1H), 6.70-6.67 (m, 1H), 6.50-6.44 (m, 2H). LCMS (ESI): 303 (M+H)

Step 3: tert-butyl (1,8-dibromoisoquinolin-3-yl)carbamate

The mixture of 1,8-dibromoisoquinolin-3-amine (0.94 g) and di-tert butyldicarbonate (7.2 mL) was stirred at 110 degree C. overnight. The reaction mixture was cooled to 0 degree C. and quenched with dimethylamine (2.0M in THF, 1.5 mL). The mixture was diluted with CHCl₃ and water, extracted with CHCl₃. The organic phase was washed with brine, dried over Na2SO4, filtered, concentrated in vacuo. The residue was purified by column chromatography on silica gel to give tert-butyl (1,8-dibromoisoquinolin-3-yl)carbamate (0.77 g).

1H NMR (400 MHz, CHLOROFORM-d) δ=8.19-8.15 (m, 1H), 7.88-7.85 (m, 1H), 7.77-7.73 (m, 1H), 7.64-7.53 (m, 1H), 7.40-7.31 (m, 1H), 1.56 (s, 9H)

### Preparation 18: tert-butyl 3-(2-(((R)-1-methylpyrrolidin-2-yl)methoxy)-5,6,7,8-tetrahydropyrido[3,4-d]pyrimidin-4-yl)-3,8-diazabicyclo[3.2.1]octane-8-carboxylate

The title compound was obtained in accordance with Preparation 1, with the exception that (R)-(1-methylpyrrolidin-2-yl)methanol was used instead of cyclopropane-1,1-diyldimethanol ESI-MS m/z 459 (MH+)

### Preparation 19: tert-butyl 3-(2-(((2S,4R)-4-fluoro-1-methylpyrrolidin-2-yl)methoxy)-5,6,7,8-tetrahydropyrido[3,4-d]pyrimidin-4-yl)-3,8-diazabicyclo[3.2.1]octane-8-carboxylate

The title compound was obtained in accordance with Preparation 1, with the exception that ((2S,4R)-4-fluoro-1-methylpyrrolidin-2-yl)methanol was used instead of cyclopropane-1,1-diyldimethanol ESI-MS m/z 477 (MH+)

### Preparation 20: tert-butyl 3-(2-(((S)-1-methylpyrrolidin-2-yl)methoxy)-5,6,7,8-tetrahydropyrido[3,4-d]pyrimidin-4-yl)-3,8-diazabicyclo[3.2.1]octane-8-carboxylate

The title compound was obtained in accordance with Preparation 1, with the exception that (S)-(1-methylpyrrolidin-2-yl)methanol was used instead of cyclopropane-1,1-diyldimethanol

ESI-MS m/z 459 (MH+)

### Preparation 21: tert-butyl (1R,5S)-3-(2-(((2S,4R)-4-methoxy-1-methylpyrrolidin-2-yl)methoxy)-5,6,7,8-tetrahydropyrido[3,4-d]pyrimidin-4-yl)-3,8-diazabicyclo[3.2.1]octane-8-carboxylate

The title compound was obtained in accordance with Preparation 1, with the exception that ((2S,4R)-4-methoxy-1-methylpyrrolidin-2-yl)methanol was used instead of cyclopropane-1,1-diyldimethanol

ESI-MS m/z 489 (MH+)

### Preparation 22: tert-butyl (1-bromo-8-iodoisoquinolin-3-yl)carbamate

The title compound was obtained in accordance with Preparation 17, with the exception that 2-fluoro-6-iodobenzonitrile was used instead of 2-bromo-6-fluorobenzonitrile.

### EXAMPLES

### Example 1: 4-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-5-bromonaphthalen-2-ol

To a mixture of tert-butyl 3-(2-((1-((dimethylamino)methyl)cyclopropyl)methoxy)-5,6,7,8-tetrahydropyrido[3,4-d]pyrimidin-4-yl)-3,8-diazabicyclo[3.2.1]octane-8-carboxylate (15 mg, 0.032 mmol), 1,8-dibromo-3-(methoxymethoxy)naphthalene (30 mg, 0.087 mmol) and Cs2CO3 (0.095 mmol), Xantphos (4 mg, 0.007 mmol) and Pd2dba3 (3 mg, 0.003 mmol) was added at room temperature.

The mixture was then degassed and back filled with nitrogen. The mixture was stirred at 110° C. After 21 hours, the reaction mixture was filtered through celite pad and the filtrate was concentrated. The residue was purified by column chromatography on silica gel (gradient elution, 0-30% EtOAc/MeOH) to give the mixture containing a desired product and diastereomer.

The mixture was taken up in MeOH (0.3 mL, 7 mmol) and 4M HCl dioxane solution (1 mL, 4 mmol) was added at room temperature. The mixture was stirred at room temperature for 1 hour. The reaction mixture was concentrated and the residue was purified by preparative HPLC to give the title compound (2.1 mg).

### Example 2: 4-(4-(3,8-diazabicyclo[3.2.1]octan-3-yl)-2-((1-(((R)-3-fluoropyrrolidin-1-yl)methyl)cyclopropyl)methoxy)-5,8-dihydropyrido[3,4-d]pyrimidin-7(6H)-yl)-5-bromonaphthalen-2-ol

The title compound was obtained in accordance with Example 1, with the exception that Preparation 2 was used instead of Preparation 1.

### Example 3: 4-(4-(3,8-diazabicyclo[3.2.1]octan-3-yl)-2-((1-(morpholinomethyl)cyclopropyl)methoxy)-5,8-dihydropyrido[3,4-d]pyrimidin-7 (6H)-yl) -5-bromonaphthalen-2-ol

The title compound was obtained in accordance with Example 1, with the exception that Preparation 3 was used instead of Preparation 1.

### Example 4: 4-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-5-methylnaphthalen-2-ol

The title compound was obtained in accordance with Example 1, with the exception that Preparation 5 was used instead of Preparation 4.

### Example 5: 4-(4-(3,8-diazabicyclo[3.2.1]octan-3-yl)-2-((1-(((R)-3-fluoropyrrolidin-1-yl)methyl)cyclopropyl)methoxy)-5,8-dihydropyrido[3,4-d]pyrimidin-7(6H)-yl)-5-methylnaphthalen-2-ol

The title compound was obtained in accordance with Example 1, with the exception that Preparation 2 was used instead of Preparation 1 and Preparation 5 was used instead of Preparation 4.

### Example 6: 4-(4-(3,8-diazabicyclo[3.2.1]octan-3-yl)-2-((1-(morpholinomethyl)cyclopropyl)methoxy)-5,8-dihydropyrido[3,4-d]pyrimidin-7(6H)-yl)-5-methylnaphthalen-2-ol

The title compound was obtained in accordance with Example 1, with the exception that Preparation 3 was used instead of Preparation 1 and Preparation 5 was used instead of Preparation 4.

### Example 7: 4-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-5-iodonaphthalen-2-ol

To a mixture of tert-butyl 3-(2-((1-((dimethylamino)methyl)cyclopropyl)methoxy)-5,6,7,8-tetrahydropyrido[3,4-d]pyrimidin-4-yl)-3,8-diazabicyclo[3.2.1]octane-8-carboxylate (100 mg, 0.212 mmol), 1,8-dibromo-3-(methoxymethoxy) naphthalene (183 mg, 0.529 mmol) and Cs2CO3 (345 mg, 1.06 mmol) in toluene (1.00 mL), Xantphos (147 mg, 0.254 mmol) and Pd2dba3 (116 mg, 0.127 mmol) was added at room temperature. The mixture was then degassed and back filled with nitrogen. The mixture was stirred at 110° C.

After 21 hours, the reaction mixture was filtered through celite pad and the filtrate was concentrated. The residue was purified by column chromatography on silica gel (gradient elution, 0-50% EtOAc/hexane) to give the mixture containing a desired product. The mixture was taken up in dioxane (4.67 mL). To the mixture, NaI (87.4 mg, 0.583 mmol), CuI (11.1 mg, 0.0583 mmol) and N,N′-dimethylethylenediamine (0.0126 mL, 0.117 mmol) were added at room temperature. The mixture was stirred at 110° C.

After 16 hours, NaI (175 mg, 1.17 mmol), CuI (22.2 mg, 0.117 mmol) and N,N′-dimethylethylenediamine (0.0251, 0.233 mmol) were added to the reaction mixture at room temperature. After 3 hours, the reaction mixture was filtered through celite pad and the filtrate was concentrated. The residue was purified by column chromatography on silica gel (gradient elution, 0-50% EtOAc/hexane) to give the mixture containing a desired product. The mixture was taken up in methanol (0.300 mL) and 4M HCl dioxane solution (0.600 mL, 2.4 mmol) was added at room temperature.

The mixture was stirred at room temperature for 1 hour. The reaction mixture was concentrated and the residue was purified by preparative HPLC to give the title compound (11.0 mg).

### Example 8: 4-(4-(3,8-diazabicyclo[3.2.1]octan-3-yl)-2-((1-(((R)-3-fluoropyrrolidin-1-yl)methyl)cyclopropyl)methoxy)-5,8-dihydropyrido[3,4-d]pyrimidin-7(6H)-yl)-5-iodonaphthalen-2-ol

The title compound was obtained in accordance with Example 7, with the exception that preparation 2 was used instead of preparation 1.

### Example 9: 4-(4-(3,8-diazabicyclo[3.2.1]octan-3-yl)-2-((1-(morpholinomethyl)cyclopropyl)methoxy)-5,8-dihydropyrido[3,4-d]pyrimidin-7(6H)-yl)-5-iodonaphthalen-2-ol

The title compound was obtained in accordance with Example 7, with the exception that preparation 3 was used instead of preparation 1.

### Example 10: (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl) (8-ethynyl-3-hydroxynaphthalen-1-yl)methanone

### Step 1

tert-butyl 3-(6-(3-hydroxy-8-iodo-1-naphthoyl)-2-((1-(morpholinomethyl)cyclopropyl)methoxy)-6,7-dihydro-5H-pyrrolo[3,4-d]pyrimidin-4-yl)-3,8-diazabicyclo[3.2.1]octane-8-carboxylate

The mixture of tert-butyl 3-(2-((1-(morpholinomethyl)cyclopropyl)methoxy)-6,7-dihydro-5H-pyrrolo[3,4-d]pyrimidin-4-yl)-3,8-diazabicyclo[3.2.1]octane-8-carboxylate (120 mg, 0.240 mmol), 3-hydroxy-8-iodo-1-naphthoic acid (82.8 mg, 0.264 mmol), 1-(3-dimethylaminopropyl)-3-ethylcarbodiimide hydrochloride (50.6 mg, 0.264 mmol), 3H-[1,2,3]triazolo[4,5-b]pyridin-3-ol (35.9 mg, 0.264 mmol) and DIPEA (167 μL, 0.959 mmol) in DMF (1.2 mL) were stirred at rt for 2 h. The mixture was diluted with EtOAc and water, extracted with EtOAc.

The organic phase was washed with brine, dried over Na₂SO₄, filtered, and concentrated in vacuo. The residue was purified by column chromatography on silica gel to give a title compound (120 mg, 0.150 mmol, 63%). ESI-MS: [M+H]⁺=797.

### Step 2

tert-butyl 3-(6-(3-hydroxy-8-((triisopropylsilyl)ethynyl)-1-naphthoyl)-2-((1-(morpholinomethyl)cyclopropyl)methoxy)-6,7-dihydro-5H-pyrrolo[3,4-d]pyrimidin-4-yl)-3,8-diazabicyclo[3.2.1]octane-8-carboxylate

To a solution of tert-butyl 3-(6-(3-hydroxy-8-iodo-1-naphthoyl)-2-((1-(morpholinomethyl)cyclopropyl)methoxy)-6,7-dihydro-5H-pyrrolo[3,4-d]pyrimidin-4-yl)-3,8-diazabicyclo[3.2.1]octane-8-carboxylate (82.2 mg, 0.103 mmol), CuI (2.0 mg, 0.0103 mmol) and [1,1′-bis (diphenylphosphino)ferrocene]palladium(II) dichloride dichloromethane adduct (8.4 mg, 0.0103 mmol) in THF (2.1 mL) was added triethylamine (108 μL, 0.774 mmol) and ethynyltriisopropylsilane (116 μL, 0.516 mmol).

The mixture was stirred at 70° C. for 9 h, cooled to rt, and concentrated in vacuo. The residue was purified by column chromatography on silica gel to give a title compound (84.9 mg, 0.0997 mmol, 97%). ESI-MS: [M+H]⁺=852.

### Step 3

(4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl) (3-hydroxy-8-((triisopropylsilyl)ethynyl)naphthalen-1-yl)methanone

A solution of tert-butyl 3-(6-(3-hydroxy-8-((triisopropylsilyl)ethynyl)-1-naphthoyl)-2-((1-(morpholinomethyl)cyclopropyl)methoxy)-6,7-dihydro-5H-pyrrolo[3,4-d]pyrimidin-4-yl)-3,8-diazabicyclo[3.2.1]octane-8-carboxylate (84.9 mg, 0.0997 mmol) in HFIP (800 μL) was irradiated at 150° C. by microwave for 25 min, and concentrated in vacuo.

The residue was purified by column chromatography on silica gel to give a title compound. ESI-MS: [M+H]⁺=752.

### Step 4: (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl) (8-ethynyl-3-hydroxynaphthalen-1-yl)methanone

To a solution of (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl) (3-hydroxy-8-((triisopropylsilyl)ethynyl)naphthalen-1-yl)methanone in THF (4.1 mL) was added TBAF (1.0 M in THF, 0.15 mL, 0.155 mmol), and the mixture was stirred at rt for 15 min, solvent was removed.

The residue was purified by RP-HPLC to give a title compound (19.5 mg, 0.0328 mmol, 33% for 2 steps).

### Example 11; (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl) (8-ethynyl-3-hydroxynaphthalen-1-yl)methanone

### Step 1

tert-butyl 3-(2-((1-(((R)-3-fluoropyrrolidin-1-yl)methyl)cyclopropyl)methoxy)-6-(3-hydroxy-8-iodo-1-naphthoyl)-6,7-dihydro-5H-pyrrolo[3,4-d]pyrimidin-4-yl)-3,8-diazabicyclo[3.2.1]octane-8-carboxylate

Prepared from tert-butyl 3-(2-((1-(((R)-3-fluoropyrrolidin-1-yl)methyl)cyclopropyl)methoxy)-6-(3-hydroxy-8-iodo-1-naphthoyl)-6,7-dihydro-5H-pyrrolo[3,4-d]pyrimidin-4-yl)-3,8-diazabicyclo[3.2.1]octane-8-carboxylate (120 mg, 0.239 mmol) using same procedure as step 1 of example 10, to give a title compound (99.8 mg, 0.125 mmol, 52%). ESI-MS: [M+H]⁺=799.

### Step 2

tert-butyl 3-(2-((1-(((R)-3-fluoropyrrolidin-1-yl)methyl)cyclopropyl)methoxy)-6-(3-hydroxy-8-((triisopropylsilyl)ethynyl)-1-naphthoyl)-6,7-dihydro-5H-pyrrolo[3,4-d]pyrimidin-4-yl)-3,8-diazabicyclo[3.2.1]octane-8-carboxylate

Prepared from tert-butyl 3-(2-((1-(((R)-3-fluoropyrrolidin-1-yl)methyl)cyclopropyl)methoxy)-6-(3-hydroxy-8-iodo-1-naphthoyl)-6,7-dihydro-5H-pyrrolo[3,4-d]pyrimidin-4-yl)-3,8-diazabicyclo[3.2.1]octane-8-carboxylate (60.1 mg, 0.0752 mmol) using same procedure as step 2 of example 10, to give a title compound (57.2 mg, 0.0670 mmol, 89%). ESI-MS: [M+H]⁺=854.

### Step 3

(4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl) (3-hydroxy-8-((triisopropylsilyl)ethynyl)naphthalen-1-yl)methanone

Prepared from tert-butyl 3-(2-((1-(((R)-3-fluoropyrrolidin-1-yl)methyl)cyclopropyl)methoxy)-6-(3-hydroxy-8-((triisopropylsilyl)ethynyl)-1-naphthoyl)-6,7H-pyrrolo[3,4-d]pyrimidin-4-yl)-3,8-diazabicyclo[3.2.1]octane-8-carboxylate (57.2 mg, 0.0670 mmol) using same procedure as step 3 of example 10, to give a title compound. ESI-MS: [M+H]⁺=754.

### Step 4

(4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl) (8-ethynyl-3-hydroxynaphthalen-1-yl)methanone

Prepared from (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl) (3-hydroxy-8-((triisopropylsilyl)ethynyl)naphthalen-1-yl)methanone using same procedure as step 4 of example 10, to give a title compound (4.83 mg, 0.00809 mmol, 11% for 2 steps).

### Example 12; (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl) (3-hydroxy-8-iodonaphthalen-1-yl)methanone

### Step 1

tert-butyl 3-(2-((1-(((R)-3-fluoropyrrolidin-1-yl)methyl)cyclopropyl)methoxy)-6-(3-hydroxy-8-iodo-1-naphthoyl)-6,7-dihydro-5H-pyrrolo[3,4-d]pyrimidin-4-yl)-3,8-diazabicyclo[3.2.1]octane-8-carboxylate

Prepared from tert-butyl 3-(2-((1-(((R)-3-fluoropyrrolidin-1-yl)methyl)cyclopropyl)methoxy)-6-(3-hydroxy-8-iodo-1-naphthoyl)-6,7-dihydro-5H-pyrrolo[3,4-d]pyrimidin-4-yl)-3,8-diazabicyclo[3.2.1]octane-8-carboxylate (20 mg, 0.0400 mmol) using same procedure as step 1 of example 10, to give a title compound as a crude product without purification. ESI-MS: [M+H]⁺=799.

### Step 2

(4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl) (3-hydroxy-8-iodonaphthalen-1-yl)methanone

A solution of tert-butyl 3-(2-((1-(((R)-3-fluoropyrrolidin-1-yl)methyl)cyclopropyl)methoxy)-6-(3-hydroxy-8-iodo-1-naphthoyl)-6,7-dihydro-5H-pyrrolo[3,4-d]pyrimidin-4-yl)-3,8-diazabicyclo[3.2.1]octane-8-carboxylate in TFA (100 μL) was stirred at rt for 15 min. After TFA was removed, the residue was purified by RP-HPLC to give a title compound (7.33 mg, 0.0105 mmol, 26% for 2 steps).

### Example 13; (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl) (3-hydroxy-8-iodonaphthalen-1-yl)methanone

### Step 1: tert-butyl 3-(6-(3-hydroxy-8-iodo-1-naphthoyl)-2-((1-(morpholinomethyl)cyclopropyl)methoxy)-6,7-dihydro-5H-pyrrolo[3,4-d]pyrimidin-4-yl)-3,8-diazabicyclo[3.2.1]octane-8-carboxylate

Prepared from tert-butyl 3-(2-((1-(morpholinomethyl)cyclopropyl)methoxy)-6,7-dihydro-5H-pyrrolo[3,4-d]pyrimidin-4-yl)-3,8-diazabicyclo[3.2.1]octane-8-carboxylate (20 mg, 0.0400 mmol) using same procedure as step 1 of example 10, to give a title compound as a crude product without purification. ESI-MS: [M+H]⁺=797.

### Step 2: (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl) (3-hydroxy-8-iodonaphthalen-1-yl) methanone

Prepared from tert-butyl 3-(6-(3-hydroxy-8-iodo-1-naphthoyl)-2-((1-(morpholinomethyl)cyclopropyl)methoxy)-6,7-dihydro-5H-pyrrolo[3,4-d]pyrimidin-4-yl)-3,8-diazabicyclo[3.2.1]octane-8-carboxylate using same procedure as step 2 of example 12, to give a title compound (9.40 mg, 0.0135 mmol, 34% for 2 steps).

### Example 14: 4-(4-(3,8-diazabicyclo[3.2.1]octan-3-yl)-6-chloro-2-((1-((dimethylamino)methyl)cyclopropyl)methoxy)-8-fluoroquinazolin-7-yl)naphthalen-2-ol

(step-1) Synthesis of tert-butyl 3-(7-bromo-2,6-dichloro-8-fluoroquinazolin-4-yl)-3,8-diazabicyclo[3.2.1]octane-8-carboxylate

To a solution of 7-bromo-2,4,6-trichloro-8-fluoro-quinazoline (2.0 g, 6.05 mmol) in 1,4-dioxane (40 mL) were added tert-butyl 3,8-diazabicyclo[3.2.1]octane-8-carboxylate (1.02 g, 4.84 mmol) and N,N-diisopropylethylamine (5.27 mL, 30.3 mmol) at room temperature. The mixture was stirred at same temperature for 30 min. Water (200 mL) was added and the precipitated solid was collected by filtration, washed with water and EtOAc, and then dried to obtain the title compound as a yellow solid. (3.30 g). ESI-MS: [M+H]⁺=507.

### (Step-2) Synthesis of tert-butyl 3-[7-bromo-6-chloro-2-[[1-[(dimethylamino)methyl]cyclopropyl]methoxy]-8-fluoro-quinazolin-4-yl]-3,8-diazabicyclo[3.2.1]octane-8-carboxylate

To a solution of tert-butyl 3-(7-bromo-2,6-dichloro-8-fluoro-quinazolin-4-yl)-3,8-diazabicyclo[3.2.1]octane-8-carboxylate (100 mg, 0.198 mmol) in 1,4-dioxane (2.0 mL) were added [1-[(dimethylamino)methyl]cyclopropyl]methanol (51 mg, 0.395 mmol) and cesium carbonate (193 mg, 0.593 mmol) at room temperature. The mixture was stirred at 140° C. for 3 h. Water was added and the mixture was extracted with EtOAc.

The organic layer was washed with water and brine, dried over anhydrous Na₂SO₄, filtered, and concentrated in vacuo. The residue was purified by column chromatography on silica gel (gradient elution, 00-20% MeOH/EtOAc) to give the title compound (60 mg). ESI-MS: [M+H]⁺=600.

### (Step-3) Synthesis of tert-butyl 3-[6-chloro-2-[[1-[(dimethylamino)methyl]cyclopropyl]methoxy]-8-fluoro-7-(3-hydroxy-1-naphthyl)quinazolin-4-yl]-3,8-diazabicyclo[3.2.1]octane-8-carboxylate

To a solution of tert-butyl 3-[7-bromo-6-chloro-2-[[1-[(dimethylamino)methyl]cyclopropyl]methoxy]-8-fluoro-quinazolin-4-yl]-3,8-diazabicyclo[3.2.1]octane-8-carboxylate (60 mg, 0.0995 mmol) in 1,4-dioxane (1.0 mL) were added 4-(4,4,5,5-tetramethyl-1,3,2-dioxaborolan-2-yl)naphthalen-2-ol (40 mg, 0.149 mmol), 2M sodium carbonate solution in water (0.5 mL, 0.995 mmol) and tetrakis(triphenylphosphine)palladium(0) (5.7 mg, 0.00498 mmol) at room temperature.

The mixture was stirred at 100° C. for 2 h. Then the reaction mixture was cooled to room temperature, and filtered through celite. and washed with MeOH/CHCl3. The filtrate was concentrated in vacuo to give the title compound. ESI-MS:

[M+H]⁺=662.

### (Step-4) Synthesis of Example 14

To a solution of tert-butyl 3-[6-chloro-2-[[1-[(dimethylamino)methyl]cyclopropyl]methoxy]-8-fluoro-7-(3-hydroxy-1-naphthyl)quinazolin-4-yl]-3,8-diazabicyclo[3.2.1]octane-8-carboxylate in chloroform (1.0 mL) were added trifluoroacetic acid (1 mL) at room temperature.

The mixture was stirred at same temperature for 30 min. After concentration, the residue was purified by r-HPLC. The obtained fractions were passed through Vari-Pure, and concentrated in vacuo to give the title compound (20.9 mg).

### Example 15: 4-(4-(3,8-diazabicyclo[3.2.1]octan-3-yl)-2-((1-((dimethylamino) methyl) cyclopropyl) methoxy)-8-fluoroquinazolin-7-yl) naphthalen-2-ol

### (Step-1) Synthesis of tert-butyl 3-(7-bromo-2-chloro-8-fluoro-quinazolin-4-yl)-8-azabicyclo[3.2.1]octane-8-carboxylate

Prepared from 7-bromo-2,4-dichloro-8-fluoro-quinazoline (850 mg, 2.87 mmol) using same procedure as step 1 of example 14, to give the title compound (550 mg, 1.16 mmol, 41%) ESI-MS: [M+H]⁺=473.

### (Step-2) Synthesis of tert-butyl 3-[7-bromo-2-[[1-[(dimethylamino)methyl]cyclopropyl]methoxy]-8-fluoro-quinazolin-4-yl]-3,8-diazabicyclo[3.2.1]octane-8-carboxylate

Prepared from tert-butyl 3-(7-bromo-2-chloro-8-fluoro-quinazolin-4-yl)-3,8-diazabicyclo[3.2.1]octane-8-carboxylate (75 mg, 0.159 mmol) using same procedure as step 2 of example 14, to give the title compound as a crude sample. ESI-MS: [M+H]⁺=566.

### (Step-3) Synthesis of tert-butyl 3-[2-[[1-[(dimethylamino)methyl]cyclopropyl]methoxy]-8-fluoro-7-(3-hydroxy-1-naphthyl)quinazolin-4-yl]-3,8-diazabicyclo[3.2.1]octane-8-carboxylate

Prepared from tert-butyl 3-[7-bromo-2-[[1-[(dimethylamino)methyl]cyclopropyl]methoxy]-8-fluoro-quinazolin-4-yl]-3,8-diazabicyclo[3.2.1]octane-8-carboxylate using same procedure as step 3 of example 14, to give the title compound (38 mg, 0.061 mmol, 2 steps 38%).

ESI-MS: [M+H]⁺=628.

### (Step-4) Synthesis of Example 15

Prepared from tert-butyl 3-[2-[[1-[(dimethylamino)methyl]cyclopropyl]methoxy]-8-fluoro-7-(3-hydroxy-1-naphthyl)quinazolin-4-yl]-3,8-diazabicyclo[3.2.1]octane-8-carboxylate (38 mg, 0.061 mmol) using same procedure as step 4 of example 14, to give the title compound (22 mg, 0.041 mmol, 68%).

### Example 16: 4-(4-(3,8-diazabicyclo[3.2.1]octan-3-yl)-8-fluoro-2-(((S)-1-methylpyrrolidin-2-yl)methoxy) quinazolin-7-yl) naphthalen-2-ol

### (Step-1) Synthesis of tert-butyl 3-[7-bromo-8-fluoro-2-[[(2S)-1-methylpyrrolidin-2-yl]methoxy]quinazolin-4-yl]-3,8-diazabicyclo[3.2.1]octane-8-carboxylate

Prepared from tert-butyl 3-(7-bromo-2-chloro-8-fluoro-quinazolin-4-yl)-3,8-diazabicyclo[3.2.1]octane-8-carboxylate (75 mg, 0.159 mmol) and [(2S)-1-methylpyrrolidin-2-yl]methanol (37 mg, 0.32 mmol) using same procedure as step 2 of example 14, to give the title compound as a crude sample. ESI-MS: [M+H]⁺=550.

### (Step-2) Synthesis of tert-butyl 3-[8-fluoro-7-(3-hydroxy-1-naphthyl)-2-[[(2S)-1-methylpyrrolidin-2-yl]methoxy]quinazolin-4-yl]-3,8-diazabicyclo[3.2.1]octane-8-carboxylate

Prepared from tert-butyl 3-[7-bromo-8-fluoro-2-[[(2S)-1-methylpyrrolidin-2-yl]methoxy]quinazolin-4-yl]-3,8-diazabicyclo[3.2.1]octane-8-carboxylate using same procedure as step 3 of example 14, to give the title compound (31 mg, 0.050 mmol, 2 steps 32%). ESI-MS: [M+H]⁺=614.

### (Step-3) Synthesis of Example 16

Prepared from tert-butyl 3-[8-fluoro-7-(3-hydroxy-1-naphthyl)-2-[[(2S)-1-methylpyrrolidin-2-yl]methoxy]quinazolin-4-yl]-3,8-diazabicyclo[3.2.1]octane-8-carboxylate (31 mg, 0.050 mmol) using same procedure as step 4 of example 14, to give the title compound (22 mg, 0.044 mmol, 86%).

### Example 17: 4-(4-(3,8-diazabicyclo[3.2.1]octan-3-yl)-2-((cis-2-(dimethylamino)cyclobutyl)methoxy)-8-fluoroquinazolin-7-yl)naphthalen-2-ol

### (Step-1) Synthesis of tert-butyl 3-[7-bromo-2-[[cis-2-(dimethylamino)cyclobutyl]methoxy]-8-fluoro-quinazolin-4-yl]-3,8-diazabicyclo[3.2.1]octane-8-carboxylate

Prepared from tert-butyl 3-(7-bromo-2-chloro-8-fluoro-quinazolin-4-yl)-3,8-diazabicyclo[3.2.1]octane-8-carboxylate (200 mg, 0.424 mmol) and [cis-2-(dimethylamino)cyclobutyl]methanol (164 mg, 1.27 mmol) using same procedure as step 2 of example 14, to give the title compound (80 mg, 0.142 mmol, 33%). ESI-MS: [M+H]⁺=566.

### (Step-2) Synthesis of tert-butyl 3-[2-[[cis-2-(dimethylamino)cyclobutyl]methoxy]-8-fluoro-7-(3-hydroxy-1-naphthyl)quinazolin-4-yl]-3,8-diazabicyclo[3.2.1]octane-8-carboxylate

Prepared from tert-butyl 3-[7-bromo-2-[[cis-2-(dimethylamino)cyclobutyl]methoxy]-8-fluoro-quinazolin-4-yl]-3,8-diazabicyclo[3.2.1]octane-8-carboxylate (80 mg, 0.142 mmol) and using same procedure as step 3 of example 14, to give the title compound (32 mg, 0.0509 mmol, 36%).

ESI-MS: [M+H]⁺=628.

### (Step-3) Synthesis of example 17

Prepared from tert-butyl 3-[2-[[cis-2-(dimethylamino)cyclobutyl]methoxy]-8-fluoro-7-(3-hydroxy-1-naphthyl)quinazolin-4-yl]-3,8-diazabicyclo[3.2.1]octane-8-carboxylate (15 mg, 0.0239 mmol) using same procedure as step 4 of example 14, to give the title compound (5.29 mg, 0.0100 mmol, 42%).

### Example 18: 4-(4-(3,8-diazabicyclo[3.2.1]octan-3-yl)-2-((1-((dimethylamino)methyl)cyclopropyl)methoxy)-6,8-difluoroquinazolin-7-yl)naphthalen-2-ol

### (Step-1) Synthesis of tert-butyl 3-(7-bromo-2-chloro-6,8-difluoro-quinazolin-4-yl)-3,8-diazabicyclo[3.2.1]octane-8-carboxylate

Prepared from 7-bromo-2,4-dichloro-6,8-difluoro-quinazoline (500 mg, 1.59 mmol) using same procedure as step 1 of example 14, to give the title compound (710 mg, 1.45 mmol, 91%) ESI-MS: [M+H]⁺=491.

### (Step-2) Synthesis of tert-butyl 3-[7-bromo-2-[[1-[(dimethylamino)methyl]cyclopropyl]methoxy]-6,8-difluoro-quinazolin-4-yl]-3,8-diazabicyclo[3.2.1]octane-8-carboxylate

Prepared from tert-butyl 3-(7-bromo-2-chloro-6,8-difluoro-quinazolin-4-yl)-3,8-diazabicyclo[3.2.1]octane-8-carboxylate (100 mg, 0.205 mmol) using same procedure as step 2 of example 14, to give the title compound (20 mg, 0.0343 mmol, 18%). ESI-MS: [M+H]⁺=584.

### (Step-3) Synthesis of tert-butyl 3-[2-[[1-[(dimethylamino)methyl]cyclopropyl]methoxy]-6,8-difluoro-7-(3-hydroxy-1-naphthyl)quinazolin-4-yl]-3,8-diazabicyclo[3.2.1]octane-8-carboxylate

Prepared from tert-butyl 3-[7-bromo-2-[[1-[(dimethylamino)methyl]cyclopropyl]methoxy]-6,8-difluoro-quinazolin-4-yl]-3,8-diazabicyclo[3.2.1]octane-8-carboxylate (20 mg, 0.0343 mmol) using same procedure as step 3 of example 14, to give the title compound as crude sample. ESI-MS: [M+H]⁺=646.

### (Step-4) Synthesis of Example 18

Prepared from tert-butyl 3-[2-[[1-[(dimethylamino)methyl]cyclopropyl]methoxy]-6,8-difluoro-7-(3-hydroxy-1-naphthyl)quinazolin-4-yl]-3,8-diazabicyclo[3.2.1]octane-8-carboxylate using same procedure as step 4 of example 14, to give the title compound (17.5 mg, 0.0321 mmol, 93%).

### Example 19: 4-(4-(3,8-diazabicyclo[3.2.1]octan-3-yl)-2-((1-((dimethylamino)methyl) cyclopropyl)methoxy)-6-ethyl-8-fluoroquinazolin-7-yl)naphthalen-2-ol

### (Step-1) Synthesis of 7-bromo-2,4-dichloro-8-fluoro-6-iodo-quinazoline 7-bromo-2,4-dichloro-8-fluoro-6-iodo-quinazoline was synthesized by the method described in the pamphlet of

International Publication No. WO 2018/143315

### (Step-2) Synthesis of tert-butyl 3-(7-bromo-2-chloro-8-fluoro-6-iodo-quinazolin-4-yl)-3,8-diazabicyclo[3.2.1]octane-8-carboxylate

Prepared from 7-bromo-2,4-dichloro-8-fluoro-6-iodo-quinazoline (5.30 g, 12.6 mmol) using same procedure as step 1 of example 14, to give the title compound (3.67 g, 6.14 mmol, 49%). ESI-MS: [M+H]⁺=599.

### (Step-3) Synthesis of tert-butyl 3-(7-bromo-2-chloro-8-fluoro-6-vinyl-quinazolin-4-yl)-3,8-diazabicyclo[3.2.1]octane-8-carboxylate

To a solution of tert-butyl 3-(7-bromo-2-chloro-8-fluoro-6-iodo-quinazolin-4-yl)-3,8-diazabicyclo[3.2.1]octane-8-carboxylate (3.67 g, 6.14 mmol) in THF (62 mL) were added potassium; trifluoro(vinyl)boranuide (905 mg, 6.76 mmol), 1M sodium carbonate solution in water (31 mL, 30.7 mmol) and [1,1-Bis (diphenylphosphino)ferrocene]dichloropalladium(II), complex with dichloromethane) (225 mg, 0.307 mmol) at room temperature.

The mixture was stirred at 60° C. for 5 h. Then the reaction mixture was cooled to room temperature, and extracted with EtOAc. The organic layer was washed with water and brine, dried over anhydrous Na₂SO₄, filtered, and concentrated in vacuo. The residue was purified by column chromatography on silica gel (gradient elution, 00-50% EtOAc/hexane) to give the title compound (2.54 g, 5.10 mmol, 83%). ESI-MS: [M+H]⁺=499.

### (Step-4) Synthesis of tert-butyl 3-[2-chloro-8-fluoro-7-[3-(methoxymethoxy)-1-naphthyl]-6-vinyl-quinazolin-4-yl]-3,8-diazabicyclo[3.2.1]octane-8-carboxylate

To a solution of tert-butyl 3-(7-bromo-2-chloro-8-fluoro-6-vinyl-quinazolin-4-yl)-3,8-diazabicyclo[3.2.1]octane-8-carboxylate (250 mg, 0.502 mmol) in THF (5 mL) were added 2-[3-(methoxymethoxy)-1-naphthyl]-4,4,5,5-tetramethyl-1,3,2-dioxaborolane (142 mg, 0.450 mmol), 2M sodium carbonate solution in water (2.5 mL, 0.502 mmol) and [1,1′-Bis (diphenylphosphino)ferrocene]dichloropalladium(II), complex with dichloromethane) (8.1 mg, 0.0116 mmol) at room temperature.

The mixture was stirred at 100° C. for 1 h. Then the reaction mixture was cooled to room temperature, and extracted with EtOAc. The organic layer was washed with water and brine, dried over anhydrous Na₂SO₄, filtered, and concentrated in vacuo. The residue was purified by column chromatography on silica gel (gradient elution, 10-80% EtOAc/hexane) to give the title compound (185 mg, 0.305 mmol, 61%). ESI-MS: [M+H]⁺=605.

### (Step-5) Synthesis of tert-butyl 3-[2-[[1-[(dimethylamino)methyl]cyclopropyl]methoxy]-8-fluoro-7-[3-(methoxymethoxy)-1-naphthyl]-6-vinyl-quinazolin-4-yl]-3,8-diazabicyclo[3.2.1]octane-8-carboxylate

Prepared from tert-butyl 3-[2-chloro-8-fluoro-7-[3-(methoxymethoxy)-1-naphthyl]-6-vinyl-quinazolin-4-yl]-3,8-diazabicyclo[3.2.1]octane-8-carboxylate (135 mg, 0.223 mmol) using same procedure as step 2 of example 14, to give the title compound (40 mg, 0.0573 mmol, 26%). ESI-MS: [M+H]⁺=698.

### (Step-6) Synthesis of tert-butyl 3-[2-[[1-[(dimethylamino)methyl]cyclopropyl]methoxy]-6-ethyl-8-fluoro-7-[3-(methoxymethoxy)-1-naphthyl]quinazolin-4-yl]-3,8-diazabicyclo[3.2.1]octane-8-carboxylate

To a solution of tert-butyl 3-[2-[[1-[(dimethylamino)methyl]cyclopropyl]methoxy]-8-fluoro-7-[3-(methoxymethoxy)-1-naphthyl]-6-vinyl-quinazolin-4-yl]-3,8-diazabicyclo[3.2.1]octane-8-carboxylate (40 mg, 0.0573 mmol) in methanol (2 mL) were added palladium on carbon (5 mg, 0.450 mmol) at room temperature.

The mixture was stirred at same temperature for 3 h under hydrogen atmosphere. Palladium was removed via filteration. The filtrate was concentrated in vacuo to give the title compound as a crude sample (40 mg, 0.0572 mmol, 99%). ESI-MS: [M+H]⁺=700.

### (Step-7) Synthesis of example 19

Prepared from tert-butyl 3-[2-[[1-[(dimethylamino)methyl]cyclopropyl]methoxy]-6-ethyl-8-fluoro-7-[3-(methoxymethoxy)-1-naphthyl]quinazolin-4-yl]-3,8-diazabicyclo[3.2.1]octane-8-carboxylate (19 mg, 0.0271 mmol) using same procedure as step 4 of example 14, to give the title compound (4.11 mg, 0.00740 mmol, 27%).

### Example 20: 4-(4-((1S,4S)-2,5-diazabicyclo[2.2.2]octan-2-yl)-2-((1-((dimethylamino)methyl)cyclopropyl)methoxy)-5,8-dihydropyrido[3,4-d]pyrimidin-7(6H)-yl)-5-bromonaphthalen-2-ol

The title compound was obtained in accordance with Example 1, with the exception that Preparation 13 was used instead of Preparation 1.

### Example 21: 4-(4-(3,8-diazabicyclo[3.2.1]octan-3-yl)-2-((1-((dimethylamino)methyl)-2,2-difluorocyclopropyl)methoxy)-5,8-dihydropyrido[3,4-d]pyrimidin-7(6H)-yl)-5-bromonaphthalen-2-ol

The title compound was obtained in accordance with Example 1, with the exception that Preparation 14 was used instead of Preparation 1.

### Example 22: 1-(4-(3,8-diazabicyclo[3.2.1]octan-3-yl)-2-((1-((dimethylamino) methyl) cyclopropyl) methoxy)-5,8-dihydropyrido[3,4-d]pyrimidin-7 (6H)-yl) -8-bromoisoquinolin-3-amine

To a mixture of Preparation 1 (60 mg) and Preparation 17 (77 mg) in DMF (0.6 mL) was added DIEPA (0.088 mL) and stirred at 50 degree C. for 3h. The mixture was diluted with EtOAc and water, extracted with EtOAc. The organic phase was washed with brine, dried over Na₂SO₄, filtered, concentrated in vacuo. The residue was used without further purification. To a solution of the residue in CHCl₃ (0.3 mL) was added TFA (0.3 mL) and stirred at room temperature for 30 min. The mixture was diluted with CHCl₃ and sat. NaHCO₃ aq., extracted with CHCl₃. The organic phase was washed with brine, dried over Na₂SO₄, filtered, concentrated in vacuo. The residue was purified by RP-HPLC to give the title compound (7.9 mg) as a solid.

### Example 23: 4-(4-(3,8-diazabicyclo[3.2.1]octan-3-yl)-2-((1-((dimethylamino) methyl) cyclopropyl) methoxy)-7,8-dihydro-5H-pyrano[4,3-d]pyrimidin-7-yl)-5-ethynylnaphthalen-2-ol in Scheme 4

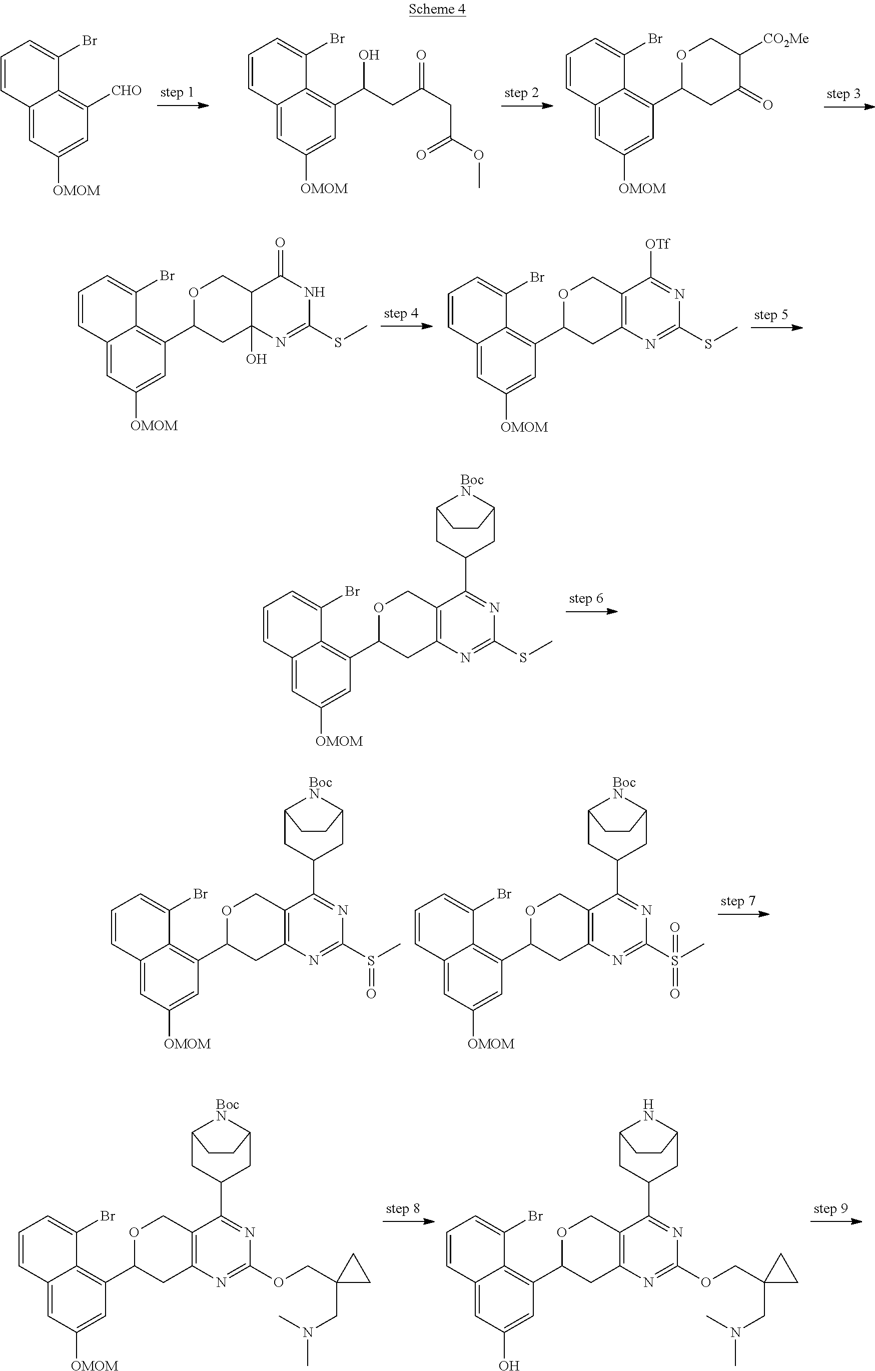

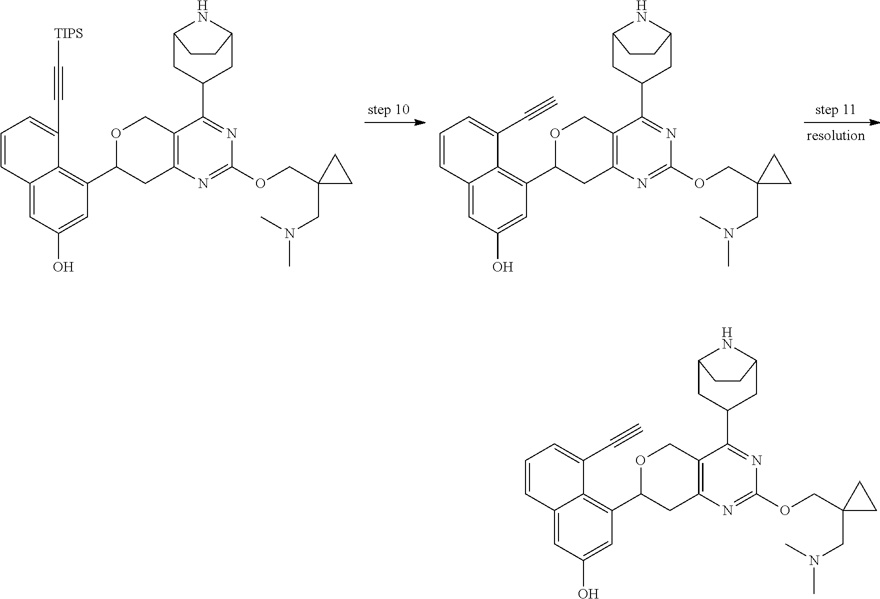

### Step 1: methyl 5-(8-bromo-3-(methoxymethoxy)naphthalen-1-yl)-5-hydroxy-3-oxopentanoate

To a solution of methyl acetoacetate (1.5 mL, 14 mmol) in THF (40 mL) was added sodium hydride (0.56 g, 14 mmol, 60% dispersion in paraffin liquid) at 0° C., the mixture was stirred for 30 min at 0° C. To the mixture was dropwise added n-butyl lithium (8.9 mL, 14 mmol, 1.6 M in hexane) at −15° C., and the mixture was stirred for an hour at the same temperature. To the mixture was added 8-bromo-3-(methoxymethoxy)-1-naphthaldehyde (2.6 g, 8.8 mmol) at −15° C., and the mixture was stirred for 30 min at the same temperature. To the mixture was added saturated aqueous NH4Cl solution, and diluted with EtOAc. The organic layer was separated and washed with H2O and concentrated. The residue was purified by column chromatography on silica gel (gradient elution, 20-60% EtOAc/Hexane) to give the title compound (3.4 g).

ESI-MS m/z 430 (MH30+)

### Step 2: methyl 6-(8-bromo-3-(methoxymethoxy)naphthalen-1-yl)-4-oxotetrahydro-2H-pyran-3-carboxylate

To a solution of methyl 5-(8-bromo-3-(methoxymethoxy)naphthalen-1-yl)-5-hydroxy-3-oxopentanoate (3.0 g, 7.2 mmol) in dichloromethane (36 mL) was added N,N-dimethylformamide dimethyl acetal (1.1 mL, 8.0 mmol) at room temperature, the mixture was stirred for 2 hours. To the mixture was added boron trifluoride-ethyl ether complex (0.91 mL, 7.2 mmol) at 0° C., and then the mixture was diluted with EtOAc. After removal of dichloromethane under vacuo, saturated aqueous NaHCO₃solution was added to the mixture, and the organic layer was separated and washed with H2O and concentrated. The residue was used for next reaction without purification.

To a solution of the above product in THF (36 mL) was added L-Selectride solution (7.2 mL, 7.2 mmol, 1M in THF) at −78° C., the mixture was stirred for at the same temperature. Additional L-Selectride solution (0.5 mL) was added, and then saturated aqueous NH4Cl solution was added. The mixture was extracted with EtOAc, and the organic layer was separated and washed with H2O and concentrated. The residue was purified by column chromatography on silica gel (gradient elution, 0-30% EtOAc/Hexane) to give the title compound (2.3 g)

ESI-MS m/z 423 (MH+)

### Step 3: 7-(8-bromo-3-(methoxymethoxy)naphthalen-1-yl)-8a-hydroxy-2-(methylthio)-3,4a,5,7,8,8a-hexahydro-4H-pyrano[4,3-d]pyrimidin-4-one

To a solution of methyl 6-(8-bromo-3-(methoxymethoxy)naphthalen-1-yl)-4-oxotetrahydro-2H-pyran-3-carboxylate (2.7 g, 5.3 mmol) and S-methylisothiourea sulfate (3.0 g, 11 mmol) in MeOH (60 mL) and THF (20 mL) was added sodium methoxide (1.4 g, 27 mmol) at room temperature, the mixture was stirred for 2 hours at the same temperature. Solvent was removed using rotavap, and dichloromethane was added to the residue. The mixture was washed with H2O and concentrated to give the title compound (2.7 g).

ESI-MS m/z 481 (MH+)

### Step 4: 7-(8-bromo-3-(methoxymethoxy)naphthalen-1-yl)-2-(methylthio)-7,8-dihydro-5H-pyrano[4,3-d]pyrimidin-4-yl trifluoromethanesulfonate

To a solution of 7-(8-bromo-3-(methoxymethoxy)naphthalen-1-yl)-8a-hydroxy-2-(methylthio)-3,4a,5,7,8,8a-hexahydro-4H-pyrano[4,3-d]pyrimidin-4-one (1.0 g, 2.1 mmol) in 2,6-lutidine (20 mL) was added trifluoromethanesulfonic anhydride (2.1 mL, 12 mmol) at −10° C., and the mixture was stirred for 10 min at room temperature. After cooling to −10° C., additional trifluoromethanesulfonic anhydride (2.1 mL, 12 mmol) was added, and the mixture was allowed to warm to room temperature. After the reaction was completed, to the mixture was added EtOAc and aqueous HCl solution (1M), and extracted with EtOAc. The organic layer was separated and washed with aqueous HCl solution (1M) and H2O, and concentrated. The residue was purified by column chromatography on silica gel (gradient elution, 0-30% EtOAc/Hexane) to give the title compound (0.80 g).

ESI-MS m/z 595 (MH+)

### Step 5: tert-butyl 3-(7-(8-bromo-3-(methoxymethoxy)naphthalen-1-yl)-2-(methylthio)-7,8-dihydro-5H-pyrano[4,3-d]pyrimidin-4-yl)-3,8-diazabicyclo[3.2.1]octane-8-carboxylate

To a solution of 7-(8-bromo-3-(methoxymethoxy)naphthalen-1-yl)-2-(methylthio)-7,8-dihydro-5H-pyrano[4,3-d]pyrimidin-4-yl trifluoromethanesulfonate (1.2 g, 2.0 mmol) in DMA (12 mL) was added tert-butyl 3,8-diazabicyclo[3.2.1]octane-8-carboxylate (0.56 g, 0.51 mmol) and N,N-diisopropylethylamine (0.71 mL, 4.1 mmol) at room temperature. After stirring at 100° C. for 10 min, the reaction mixture was diluted with EtOAc and saturated aqueous NH4Cl solution. The organic layer was separated and washed with H2O and concentrated. The residue was purified by column chromatography on silica gel (gradient elution, 0-50% EtOAc/Hexane) to give the title compound (1.2 g).

ESI-MS m/z 657 (MH+)

### Step 6

tert-butyl 3-(7-(8-bromo-3-(methoxymethoxy)naphthalen-1-yl)-2-(methylsulfinyl)-7,8-dihydro-5H-pyrano[4,3-d]pyrimidin-4-yl)-3,8-diazabicyclo[3.2.1]octane-8-carboxylate and tert-butyl 3-(7-(8-bromo-3-(methoxymethoxy)naphthalen-1-yl)-2-(methylsulfonyl)-7,8-dihydro-5H-pyrano[4,3-d]pyrimidin-4-yl)-3,8-diazabicyclo[3.2.1]octane-8-carboxylate

To a solution of tert-butyl 3-(7-(8-bromo-3-(methoxymethoxy)naphthalen-1-yl)-2-(methylthio)-7,8-dihydro-5H-pyrano[4,3-d]pyrimidin-4-yl)-3,8-diazabicyclo[3.2.1]octane-8-carboxylate (0.58 g, 0.88 mmol) in EtOAc (20 mL) was added m-chloroperoxybenzoic acid (0.20 g, 0.88 mmol, with abt. 25% water) at 0° C., the mixture was stirred for an hour at the same temperature. To the mixture was added aqueous NaHCO₃ solution, and the organic layer was separated and washed with H2O and concentrated. The residue was purified by column chromatography on silica gel (gradient elution, 60-100% EtOAc/Hexane, 0-20% MeOH/EtOAc) to give the title sulfoxide (0.53 g) and sulfone (38 mg).

ESI-MS m/z 673 (MH+): sulfoxide

ESI-MS m/z 689 (MH+): sulfone

### Step 7

tert-butyl 3-(7-(8-bromo-3-(methoxymethoxy)naphthalen-1-yl)-2-((1-((dimethylamino)methyl)cyclopropyl)methoxy)-7,8-dihydro-5H-pyrano[4,3-d]pyrimidin-4-yl)-3,8-diazabicyclo[3.2.1]octane-8-carboxylate

To a solution of tert-butyl 3-(7-(8-bromo-3-(methoxymethoxy)naphthalen-1-yl)-2-(methylsulfinyl)-7,8-dihydro-5H-pyrano[4,3-d]pyrimidin-4-yl)-3,8-diazabicyclo[3.2.1]octane-8-carboxylate (0.26 g, 0.38 mmol) and (1-[(dimethylamino)methyl]cyclopropyl)methanol (0.15 mL, 1.2 mmol) in THF (3 mL) was added sodium tert-butoxide (0.25 mL, 0.50 mmol, 2M solution in THF) at 0° C., the mixture was stirred for an hour at the same temperature. To the mixture was added EtOAc and water, and the organic layer was separated and washed with H2O and concentrated. The residue was purified by column chromatography on NH silica gel (gradient elution, 10-50% EtOAc/Hexane) to give the title compound (0.24 g).

ESI-MS m/z 738 (MH+)

### Step 8: 4-(4-(3,8-diazabicyclo[3.2.1]octan-3-yl)-2-((1-((dimethylamino)methyl)cyclopropyl)methoxy)-7,8-dihydro-5H-pyrano[4,3-d]pyrimidin-7-yl)-5-bromonaphthalen-2-ol (see Example 37)

To a solution of tert-butyl 3-(7-(8-bromo-3-(methoxymethoxy)naphthalen-1-yl)-2-((1-((dimethylamino)methyl)cyclopropyl)methoxy)-7,8-dihydro-5H-pyrano[4,3-d]pyrimidin-4-yl)-3,8-diazabicyclo[3.2.1]octane-8-carboxylate (0.24 g, 0.33 mmol) in MeOH (0.4 mL) was added HCl solution (4 mL, 16 mmol, 4 M in 1,4-dioxane) at room temperature. After stirring for 30 min, the mixture was concentrated in vacuo. The residue was purified by column chromatography on NH silica gel (gradient elution, 0-40% MeOH/EtOAc) to give the title compound (0.18 g).

### Step 9: 4-(4-((3,8-diazabicyclo[3.2.1]octan-3-yl)-2-((1-((dimethylamino)methyl)cyclopropyl)methoxy)-7,8-dihydro-5H-pyrano[4,3-d]pyrimidin-7-yl)-5-((triisopropylsilyl)ethynyl)naphthalen-2-ol

To a solution of 4-(4-(3,8-diazabicyclo[3.2.1]octan-3-yl)-2-((1-((dimethylamino)methyl)cyclopropyl)methoxy)-7,8-dihydro-5H-pyrano[4,3-d]pyrimidin-7-yl)-5-bromonaphthalen-2-ol (0.12 g, 0.20 mmol), copper(I) iodide (7.6 mg, 0.040 mmol), bis (triphenylphosphine)palladium (II) dichloride (28 mg, 0.040 mmol) and N,N-diisopropylethylamine (280 uL, 2.0 mmol) in DMA (4 mL) was added (triisopropylsilyl) acetylene (220 uL, 0.41 mmol) at room temperature. The vessel was evacuated and backfilled with nitrogen, and the mixture was stirred at 110° C. for an hour. After the reaction was completed, the mixture was diluted with EtOAc and water, and the organic layer was separated and washed with water and concentrated. The residue was purified by column chromatography on NH silica gel (gradient elution, 0-40% MeOH/EtOAc) to give the title compound (130 mg).

ESI-MS m/z 696 (MH+)

### Step 10: 4-(4-(3,8-diazabicyclo[3.2.1]octan-3-yl)-2-((1-((dimethylamino)methyl)cyclopropyl)methoxy)-7,8-dihydro-5H-pyrano[4,3-d]pyrimidin-7-yl)-5-ethynylnaphthalen-2-ol

To a solution of 4-(4-(3,8-diazabicyclo[3.2.1]octan-3-yl)-2-((1-((dimethylamino)methyl)cyclopropyl)methoxy)-7,8-dihydro-5H-pyrano[4,3-d]pyrimidin-7-yl)-5-((triisopropylsilyl)ethynyl)naphthalen-2-ol (130 mg, 0.19 mmol) in THF (2 mL) was added tetrabutylammonium fluoride (390 uL, 0.39 mmol, 1 M solution in THF) at 0° C., and the mixture was stirred for an hour at 0° C. The mixture was diluted with EtOAc and water, and the organic layer was separated and washed with water and concentrated. The residue was purified by column chromatography on NH silica gel (gradient elution, 0-40% MeOH/EtOAc) to give the title compound (100 mg).

ESI-MS m/z 540 (MH+)

Step 11: Optical Resolution Racemic 4-(4-(3,8-diazabicyclo[3.2.1]octan-3-yl)-2-((1-((dimethylamino)methyl)cyclopropyl)methoxy)-7,8-dihydro-5H-pyrano[4,3-d]pyrimidin-7-yl)-5-ethynylnaphthalen-2-ol was optically separated by chiral HPLC on chiral column (CHIRALPAK IC (4.6 mmφ×150 mm 5 um), gradient elution: Hexane/EtOH=70/30, additive: 0.1% diethylamine, flow rate: 1.0 mL/min) to give 4-(4-(3,8-diazabicyclo[3.2.1]octan-3-yl)-2-((1-((dimethylamino)methyl) cyclopropyl)methoxy)-7,8-dihydro-5H-pyrano[4,3-d]pyrimidin-7-yl)-5-ethynylnaphthalen-2-ol as chiral isomers.

### Example 24: 1-(1-(((4-(3,8-diazabicyclo[3.2.1]octan-3-yl)-7-(8-ethynylnaphthalen-1-yl)-5,6,7,8-tetrahydroquinazolin-2-yl)oxy)methyl)cyclopropyl)-N,N-dimethylmethanamine in Scheme 5

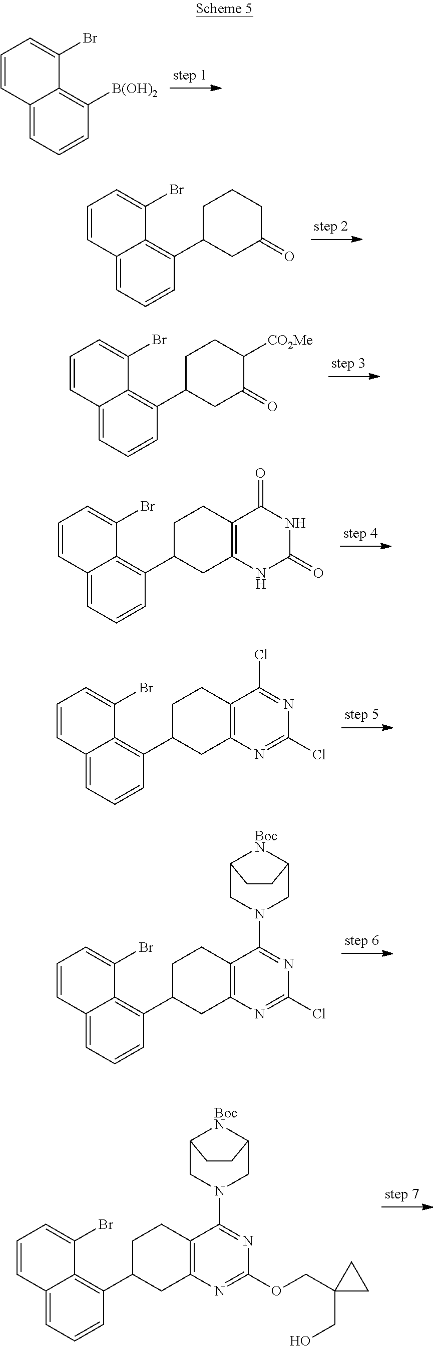

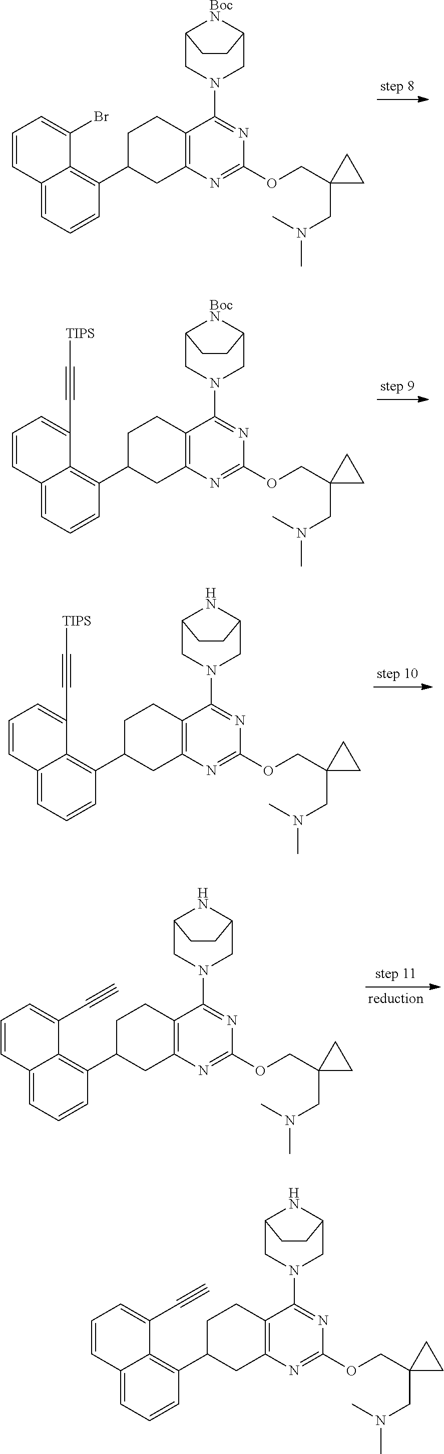

### Step 1: 3-(8-bromonaphthalen-1-yl)cyclohexan-1-one

To a solution of (8-bromonaphthalen-1-yl)boronic acid (10.0 g, 29.9 mmol) and K3PO4 (16.9 g, 79.7 mmol) in 1,4-dioxane (40 mL) and H2O (60 mL) was added 2-cyclohexen-1-one (3.81 g, 39.9 mmol) at room temperature. The mixture was degassed and backfilled with nitrogen. Hydroxy(cyclooctadiene) rhodium(I) dimer (550 mg, 1.20 mmol) was added to the mixture, and the mixture was stirred at 65° C. After an hour, K3PO4 (16.9 g, 79.7 mmol) was added to the mixture, and the mixture was stirred at 65° C. for another 1 hour. After the reaction was completed, the reaction mixture was diluted with EtOAc and the organic layer was separated. The organic layer was washed with water, and concentrated under vacuum. The residue was purified by column chromatography on silica gel (gradient elution, 5-30% EtOAc/Hexane) to give the title compound (4.04 g).

ESI-MS m/z 303 (MH+)

Step 2: methyl 4-(8-bromonaphthalen-1-yl)-2-oxocyclohexane-1-carboxylate To a solution of 3-(8-bromonaphthalen-1-yl)cyclohexan-1-one (2.0 g, 6.6 mmol) in THF (20 mL) was added lithium bis(trimethylsilyl)amide (13 mL, 13 mmol, 1.0 M in THF) dropwise at −78° C. The mixture was stirred at same temperature for 30 min, then Methyl cyanoformate (0.79 mL, 9.9 mmol) was added. The mixture was allowed to warm to 0° C., and saturated aqueous NH4Cl solution was added to the mixture. The mixture was extracted with EtOAc, and the organic layer was washed with water and concentrated. The residue was purified by column chromatography on silica gel (gradient elution, 0-25% EtOAc/Hexane) to give the title compound (1.4 g)

ESI-MS m/z 361 (MH+)

### Step 3: 7-(8-bromonaphthalen-1-yl)-5,6,7,8-tetrahydroquinazoline-2,4(1H,3H)-dione

The mixture of urea (1.8 g, 30 mmol), sodium ethoxide (2.1 g, 30 mmol) and methyl 4-(8-bromonaphthalen-1-yl)-2-oxocyclohexane-1-carboxylate (1.1 g, 3.0 mmol) in EtOH (16 mL) was irradiated at 110° C. by microwave for an hour. To the mixture was slowly added water (100 mL) and aqueous HCl solution (5.2 mL, 31 mmol, 6M). Precipitate was filtered and rinsed with H2O and dried to afford the title compound (0.68 g) as a white solid. This solid was used for next step without further purification.

ESI-MS m/z 371 (MH+)

### Step 4: 7-(8-bromonaphthalen-1-yl)-2,4-dichloro-5,6,7,8-tetrahydroquinazoline

The solution of 7-(8-bromonaphthalen-1-yl)-5,6,7,8-tetrahydroquinazoline-2,4 (1H,3H)-dione (0.85 g, 2.3 mmol) in phosphoryl chloride (51 mL) was stirred at 100° C. for 90 min. The mixture was concentrated in vacuo, the residue was purified by column chromatography on silica gel (gradient elution, 0-30% EtOAc/Hexane) to give the title compound (0.83 g) ESI-MS m/z 407 (MH+)

### Step 5: tert-butyl 3-(7-(8-bromonaphthalen-1-yl)-2-chloro-5,6,7,8-tetrahydroquinazolin-4-yl)-3,8-diazabicyclo[3.2.1]octane-8-carboxylate

To a solution of 7-(8-bromonaphthalen-1-yl)-2,4-dichloro-5,6,7,8-tetrahydroquinazoline (0.78 g, 1.9 mmol) in DMA (10 mL) was added tert-butyl 3,8-diazabicyclo[3.2.1]octane-8-carboxylate (0.43 g, 2.0 mmol) and N,N-diisopropylethylamine (0.77 mL, 3.8 mmol) at room temperature. After stirring at room temperature for 16 hours, the reaction mixture was diluted with EtOAc and saturated aqueous NH4Cl solution. The organic layer was separated and washed with H2O and concentrated. The residue was purified by column chromatography on silica gel (gradient elution, 0-40% EtOAc/Hexane) to give the title compound (0.95 g).

ESI-MS m/z 583 (MH+)

### Step 6: tert-butyl 3-(7-(8-bromonaphthalen-1-yl)-2-((1-((dimethylamino)methyl)cyclopropyl)methoxy)-5,6,7,8-tetrahydroquinazolin-4-yl)-3,8-diazabicyclo[3.2.1]octane-8-carboxylate

To a solution of tert-butyl 3-(7-(8-bromonaphthalen-1-yl)-2-chloro-5,6,7,8-tetrahydroquinazolin-4-yl)-3,8-diazabicyclo[3.2.1]octane-8-carboxylate (0.56 g, 0.96 mmol) in 1,4-dioxane (20 mL) was added 1,1-Bis(hydroxymethyl)cyclopropane (1.5 g, 1.4 mmol) and sodium tert-butoxide (0.96 mL, 1.9 mmol, 2M solution in THF) at room temperature. The mixture was stirred at 120° C. for 3 hours, then diluted with EtOAc and H2O. The organic layer was separated and washed with H2O and concentrated. The residue was purified by column chromatography on silica gel (gradient elution, 30-70% EtOAc/Hexane) to give the title compound (0.62 g).

ESI-MS m/z 649 (MH+)

### Step 7: tert-butyl 3-(7-(8-bromonaphthalen-1-yl)-2-((1-((dimethylamino)methyl)cyclopropyl)methoxy)-5,6,7,8-tetrahydroquinazolin-4-yl)-3,8-diazabicyclo[3.2.1]octane-8-carboxylate

To a solution of tert-butyl-3-(7-(8-bromonaphthalen-1-yl)-2-((1-((dimethylamino)methyl)cyclopropyl)methoxy)-5,6,7,8-tetrahydroquinazolin-4-yl)-3,8-diazabicyclo[3.2.1]octane-8-carboxylate (85 mg, 0.13 mmol) and N,N-diisopropylethylamine (140 uL, 0.79 mmol) in DMA (3 mL) was added to methanesulfonyl chloride (41 uL, 0.52 mmol) at 0° C. After stirring for 30 min at 0° C., K2CO3 (140 mg, 1.0 mmol) and dimethylamine (1.3 mL, 2.6 mmol, 2.0 M solution in THF) was added to the mixture. The mixture was stirred at 50° C. for 3 hours, and then diluted with EtOAc and water. The organic layer was separated and washed with H2O and concentrated. The residue was purified by column chromatography on NH silica gel (gradient elution, 5-40% EtOAc/Hexane) to give the title compound (73 mg).

ESI-MS m/z 676 (MH+)

### Step 8

tert-butyl 3-(2-((1-((dimethylamino)methyl)cyclopropyl)methoxy)-7-(8-((triisopropylsilyl)ethynyl)naphthalen-1-yl)-5,6,7,8-tetrahydroquinazolin-4-yl)-3,8-diazabicyclo[3.2.1]octane-8-carboxylate

To a solution of tert-butyl-3-(7-(8-bromonaphthalen-1-yl)-2-((1-((dimethylamino)methyl)cyclopropyl)methoxy)-5,6,7,8-tetrahydroquinazolin-4-yl)-3,8-diazabicyclo[3.2.1]octane-8-carboxylate (55 mg, 0.081 mmol), copper (I) iodide (3.0 mg, 0.016 mmol), bis (triphenylphosphine)palladium(II) dichloride (11 mg, 0.016 mmol) and N,N-diisopropylethylamine (110 uL, 0.81 mmol) in DMA (3 mL) was added (triisopropylsilyl)acetylene (91 uL, 0.41 mmol) at room temperature. The vessel was evacuated and backfilled with nitrogen, and the mixture was stirred at 100° C. After the reaction was completed, the mixture was diluted with EtOAc and water, and the organic layer was separated and washed with water and concentrated. The residue was purified by column chromatography on NH silica gel (gradient elution, 0-50% EtOAc/Hexane) to give the title compound (54 mg).

ESI-MS m/z 779 (MH+)

### Step 9: 1-(1-(((4-(3,8-diazabicyclo[3.2.1]octan-3-yl)-7-(8-((triisopropylsilyl)ethynyl)naphthalen-1-yl)-5,6,7,8-tetrahydroquinazolin-2-yl)oxy)methyl)cyclopropyl)-N,N-dimethylmethanamine

A solution of tert-butyl 3-(2-((1-((dimethylamino)methyl)cyclopropyl)methoxy)-7-(8-((triisopropylsilyl)ethynyl)naphthalen-1-yl)-5,6,7,8-tetrahydroquinazolin-4-yl)-3,8-diazabicyclo[3.2.1]octane-8-carboxylate (25 mg, 0.032 mmol) in hexafluoro-2-propanol (3 mL) was irradiated at 150° C. by microwave for an hour. After concentration, the residue was purified by column chromatography on NH silica gel (gradient elution, 0-20% MeOH/EtOAc) to give the title compound (13 mg).

ESI-MS m/z 679 (MH+)

### Step 10: 1-(1-(((4-(3,8-diazabicyclo[3.2.1]octan-3-yl)-7-(8-ethynylnaphthalen-1-yl)-5,6,7,8-tetrahydroquinazolin-2-yl)oxy)methyl)cyclopropyl)-N,N-dimethylmethanamine

To a solution o f1-(1-(((4-(3,8-diazabicyclo[3.2.1]octan-3-yl)-7-(8-((triisopropylsilyl)ethynyl)naphthalen-1-yl)-5,6,7,8-tetrahydroquinazolin-2-yl)oxy)methyl)cyclopropyl)-N,N-dimethylmethanamine (13 mg, 0.019 mmol) in THF (2 mL) was added tetrabutylammonium fluoride (38 uL, 0.038 mmol, 1 M solution in THF) at 0° C., and the mixture was stirred for 30 min at 0° C. The mixture was diluted with EtOAc and water, and the organic layer was separated and washed with water and concentrated. The residue was purified by column chromatography on NH silica gel (gradient elution, 0-20% MeOH/EtOAc) to give the title compound (5.0 mg).

Step 11: Optical resolution Racemic 1-(1-(((4-(3,8-diazabicyclo[3.2.1]octan-3-yl)-7-(8-ethynylnaphthalen-1-yl)-5,6,7,8-tetrahydroquinazolin-2-yl)oxy)methyl)cyclopropyl)-N,N-dimethylmethanamine was optically separated by chiral HPLC on chiral column (CHIRAL ART SB (4.6 mmφ×150 mm 5 um), gradient elution: Hexane/EtOH=80/20, additive: 0.1% diethylamine, flow rate: 1.0 mL/min) to give 1-(1-(((4-(3,8-diazabicyclo[3.2.1]octan-3-yl)-7-(8-ethynylnaphthalen-1-yl)-5,6,7,8-tetrahydroquinazolin-2-yl)oxy)methyl)cyclopropyl)-N,N-dimethylmethanamine as chiral isomers.

### Example 25: 4-((1-(((4-(3,8-diazabicyclo[3.2.1]octan-3-yl)-7-(8-iodonaphthalen-1-yl)-5,6,7,8-tetrahydropyrido[3,4-d]pyrimidin-2-yl)oxy)methyl)cyclopropyl)methyl)morpholine

The title compound was obtained in accordance with Example 9, with the exception that 1,8-dibromonaphthalene was used instead of 1,8-dibromo-3-(methoxymethoxy)naphthalene.

### Example 26: 4-(4-(3,8-diazabicyclo[3.2.1]octan-3-yl)-2-(((R)-1-methylpyrrolidin-2-yl)methoxy)-5,8-dihydropyrido[3,4-d]pyrimidin-7(6H)-yl)-5-bromonaphthalen-2-ol

The title compound was obtained in accordance with Example 1, with the exception that Preparation 18 was used instead of Preparation 1.

### Example 27: 4-(4-(3,8-diazabicyclo[3.2.1]octan-3-yl)-2-(((2S,4R)-4-fluoro-1-methylpyrrolidin-2-yl)methoxy)-5,8-dihydropyrido[3,4-d]pyrimidin-7(6H)-yl)-5-bromonaphthalen-2-ol

The title compound was obtained in accordance with Example 1, with the exception that Preparation 19 was used instead of Preparation 1.

### Example 28: 4-(4-((1S,4S)-2,5-diazabicyclo[2.2.2]octan-2-yl)-2-((1-(morpholinomethyl)cyclopropyl)methoxy)-5,8-dihydropyrido[3,4-d]pyrimidin-7(6H)-yl)-5-bromonaphthalen-2-ol

The title compound was obtained in accordance with Example 1, with the exception that Preparation 15 was used instead of Preparation 1.

### Example 29: 1-(1-(((4-(3,8-diazabicyclo[3.2.1]octan-3-yl)-7-(8-bromonaphthalen-1-yl)-5,6,7,8-tetrahydropyrido[3,4-d]pyrimidin-2-yl)oxy)methyl)-2,2-difluorocyclopropyl)-N,N-dimethylmethanamine

The title compound was obtained in accordance with Example 1, with the exception that Preparation 14 and 1,8-dibromonaphthalene was used instead of Preparation 1 and 1,8-dibromo-3-(methoxymethoxy)naphthalene.

### Example 30: 4-(4-(3,8-diazabicyclo[3.2.1]octan-3-yl)-2-((1-((dimethylamino)methyl)-2,2-dimethylcyclopropyl)methoxy)-5,8-dihydropyrido[3,4-d]pyrimidin-7(6H)-yl)-5-bromonaphthalen-2-ol

The title compound was obtained in accordance with Example 1, with the exception that Preparation 16 was used instead of Preparation 1.

### Example 31: 4-(4-(3,8-diazabicyclo[3.2.1]octan-3-yl)-2-(((S)-1-methylpyrrolidin-2-yl)methoxy)-5,8-dihydropyrido[3,4-d]pyrimidin-7(6H)-yl)-5-bromonaphthalen-2-ol

The title compound was obtained in accordance with Example 1, with the exception that Preparation 20 was used instead of Preparation 1.

### Example 32: 4-(4-(3,8-diazabicyclo[3.2.1]octan-3-yl)-2-(((2S,4R)-4-methoxy-1-methylpyrrolidin-2-yl)methoxy)-5,8-dihydropyrido[3,4-d]pyrimidin-7(6H)-yl)-5-bromonaphthalen-2-ol

The title compound was obtained in accordance with Example 1, with the exception that Preparation 21 was used instead of Preparation 1.

### Example 33: 4-(4-(3,8-diazabicyclo[3.2.1]octan-3-yl)-2-((1-(morpholinomethyl)cyclopropyl)methoxy)-7,8-dihydro-5H-pyrano[4,3-d]pyrimidin-7-yl)-5-ethynylnaphthalen-2-ol

4-(4-(3,8-diazabicyclo[3.2.1]octan-3-yl)-2-((1-(morpholinomethyl)cyclopropyl)methoxy)-7,8-dihydro-5H-pyrano[4,3-d]pyrimidin-7-yl)-5-ethynylnaphthalen-2-ol was prepared in the same manner (in step 7-10 above) using tert-butyl 3-(7-(8-bromo-3-(methoxymethoxy)naphthalen-1-yl)-2-(methylsulfonyl)-7,8-dihydro-5H-pyrano[4,3-d]pyrimidin-4-yl)-3,8-diazabicyclo[3.2.1]octane-8-carboxylate (38 mg, 0.055 mmol) and [1-(morpholinomethyl)cyclopropyl]methanol (40 mg, 0.072 mmol), instead of tert-butyl 3-(7-(8-bromo-3-(methoxymethoxy)naphthalen-1-yl)-2-(methylsulfinyl)-7,8-dihydro-5H-pyrano[4,3-d]pyrimidin-4-yl)-3,8-diazabicyclo[3.2.1]octane-8-carboxylate and (1-[(dimethylamino)methyl]cyclopropyl)methanol.

### Example 34: (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl) (3-hydroxy-8-vinylnaphthalen-1-yl)methanone

### Step 1

tert-butyl 3-(2-((1-(((R)-3-fluoropyrrolidin-1-yl)methyl)cyclopropyl)methoxy)-6-(3-hydroxy-8-vinyl-1-naphthoyl)-6,7-dihydro-5H-pyrrolo[3,4-d]pyrimidin-4-yl)-3,8-diazabicyclo[3.2.1]octane-8-carboxylate

To a solution of tert-butyl 3-(2-((1-(((R)-3-fluoropyrrolidin-1-yl)methyl)cyclopropyl)methoxy)-6-(3-hydroxy-8-iodo-1-naphthoyl)-6,7-dihydro-5H-pyrrolo[3,4-d]pyrimidin-4-yl)-3,8-diazabicyclo[3.2.1]octane-8-carboxylate (40 mg), Potassium vinyltrifluoroborate (100 mg) K3PO4(21 mg) in 1,4-dioxane (0.5 mL) and water (0.5 mL) was added Chloro(2-dicyclohexylphosphino-2,4′,6′-triisopropyl-1,1′-biphenyl) [2-(2′-amino-1,1-biphenyl)]palladium(II) (4 mg). The mixture was stirred at 100° C. for 1 h, cooled to rt, and concentrated in vacuo. The residue was purified by column chromatography on silica gel to give a title compound. ESI-MS: [M+H]⁺=699.

### Step 2: (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl) (3-hydroxy-8-vinylnaphthalen-1-yl)methanone

A solution of tert-butyl 3-(2-((1-(((R)-3-fluoropyrrolidin-1-yl)methyl)cyclopropyl)methoxy)-6-(3-hydroxy-8-vinyl-1-naphthoyl)-6,7-dihydro-5H-pyrrolo[3,4-d]pyrimidin-4-yl)-3,8-diazabicyclo[3.2.1]octane-8-carboxylate in TFA (100 μL) was stirred at rt for 15 min. After TFA was removed, the residue was purified by RP-HPLC to give a title compound (3.51 mg).

### Example 35: (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl) (3-hydroxy-8-vinylnaphthalen-1-yl) methanone

The title compound was obtained in accordance with Example 34, with the exception that tert-butyl 3-(6-(3-hydroxy-8-iodo-1-naphthoyl)-2-((1-(morpholinomethyl)cyclopropyl)methoxy)-6,7-dihydro-5H-pyrrolo[3,4-d]pyrimidin-4-yl)-3,8-diazabicyclo[3.2.1]octane-8-carboxylate was used instead of tert-butyl 3-(2-((1-(((R)-3-fluoropyrrolidin-1-yl)methyl)cyclopropyl)methoxy)-6-(3-hydroxy-8-iodo-1-naphthoyl)-6,7-dihydro-5H-pyrrolo[3,4-d]pyrimidin-4-yl)-3,8-diazabicyclo[3.2.1]octane-8-carboxylate

### Example 36: 1-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-8-ethynylisoquinolin-3-amine

### Step 1: 1-(4-((3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-8-iodoisoquinolin-3-amine

The title compound was obtained in accordance with Example 22, with the exception that Preparation 22 was used instead of Preparation 17.

### Step 2: (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl) (3-hydroxy-8-vinylnaphthalen-1-yl)methanone

To a solution of 1-(4-((3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-8-iodoisoquinolin-3-amine (35 mg), CuI (2.0 mg) and [1,1′-bis (diphenylphosphino)ferrocene]palladium(II) dichloride dichloromethane adduct (7.8 mg) in DMA (4 mL) was added triethylamine (67 μL) and ethynyltriisopropylsilane (53 μL). The mixture was stirred at room temperature for 30 min. The mixture was diluted with CHCl₃ and water, extracted with CHCl₃. The organic phase was washed with brine, dried over Na2SO4, filtered, concentrated in vacuo. To a solution of the residue in THF(2 ml) was added TBAF (0.14 mL, 1.0M THF solution). The mixture was stirred at rt for 30 min. After THF was removed, the residue was purified by RP-HPLC to give a title compound.

### Example 37: 4-(4-(3,8-diazabicyclo[3.2.1]octan-3-yl)-2-((1-((dimethylamino) methyl) cyclopropyl) methoxy)-7,8-dihydro-5H-pyrano[4,3-d]pyrimidin-7-yl)-5-bromonaphthalen-2-ol

To a solution of tert-butyl 3-(7-(8-bromo-3-(methoxymethoxy)naphthalen-1-yl)-2-((1-((dimethylamino)methyl)cyclopropyl)methoxy)-7,8-dihydro-5H-pyrano[4,3-d]pyrimidin-4-yl)-3,8-diazabicyclo[3.2.1]octane-8-carboxylate (0.24 g, 0.33 mmol) in MeOH (0.4 mL) was added HCl solution (4 mL, 16 mmol, 4 M in 1,4-dioxane) at room temperature. After stirring for 30 min, the mixture was concentrated in vacuo. The residue was purified by column chromatography on NH silica gel (gradient elution, 0-40% MeOH/EtOAc) to give the title compound (0.18 g)

Information of prepared compounds is listed in Table 1 below. Abs in the table indicates an absolute configuration.

TABLE 1

| | | | ESI-MS |
|---|---|---|---|
| NUMBER | STRUCTURE | NMR | [M + H]⁺ |
| 1 | 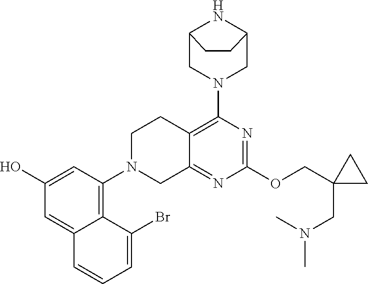 [SMILES: CN(C)CC1(COc2nc3c(c(N4CC5CCC(C4)N5)n2)CCN(c2cc(O)cc4cccc(Br)c24)C3)CC1] | 1H-NMR (400 MHz, DMSO-D6) δ: 9.93 (1H, s), 7.68 (1H, d, J = 7.5 Hz), 7.52 (1H, d, J = 7.5 Hz), 7.35 (1H, s), 7.18 (1H, dd, J = 7.5, 7.5 Hz), 6.94 (1H, d, J = 2.3 Hz), 6.86 (1H, d, J = 2.3 Hz), 4.12-3.92 (6H, m), 3.42-3.38 (4H, m), 3.30-3.28 (3H, m), 3.16 (1H, s), 3.14 (1H, s), 3.03 (1H, d, J = 9.0 Hz), 2.87 (1H, d, J = 11.5 Hz), 2.66 (1H, m), 2.54 (1H, m), 2.32 (1H, m), 2.17 (2H, d, J = 3.8 Hz), 1.81 (1H, m), 1.62 (2H, m), 1.22 (1H, brs), 0.54 (2H, m), 0.34 (2H, m). | 593 |
| 2 | 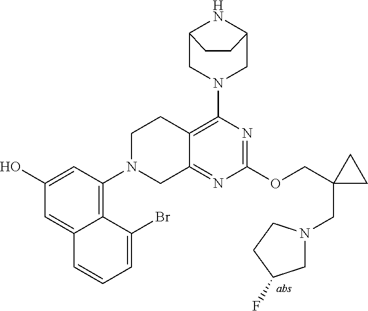 [SMILES: Oc1cc(N2CCc3c(nc(OCC4(CN5CC[C@@H](F)C5)CC4)nc3N3CC4CCC(C3)N4)C2)c2c(Br)cccc2c1] | 1H-NMR (400 MHz, CDCl3) δ: 7.59 (1H, s), 7.57 (1H, s), 7.14 (1H, t, J = 8.0 Hz), 6.90 (1H, d, J = 2.3 Hz), 6.83 (1H, d, J = 2.5 Hz), 5.21-5.05 (1H, m), 4.37 (1H, d, J = 17.8 Hz), 4.26 (1H, d, J = 10.8 Hz), 4.14 (1H, d, J = 10.8 Hz), 4.00 (1H, d, J = 12.0 Hz), 3.73 (1H, d, J = 17.8 Hz), 3.65 (1H, d, J = 12.0 Hz), 3.59 (2H, br s), 3.47-3.44 (1H, m), 3.27 (1H, d, J = 12.8 Hz), 3.14-2.98 (3H, m), 2.87-2.81 (3H, m), 2.63-2.60 (1H, m), 2.49-2.42 (3H, m), 2.16-1.97 (3H, m), 1.81 (2H, brs), 1.25 (2H, s), 0.95-0.81 (1H, m), 0.62 (2H, t, J = 5.0 Hz), 0.43 (2H, t, J = 5.0 Hz). | 639 |
| 3 | 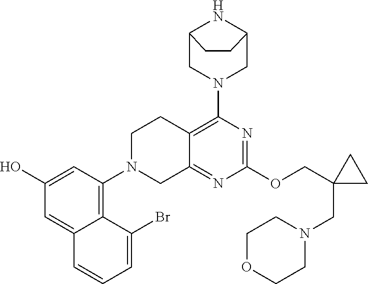 [SMILES: Oc1cc(N2CCc3c(nc(OCC4(CN5CCOCC5)CC4)nc3N3CC4CCC(C3)N4)C2)c2c(Br)cccc2c1] | 1H-NMR (400 MHz, DMSO-D6) δ: 9.93 (1H, s), 7.68 (1H, d, J = 8.3 Hz), 7.52 (1H, d, J = 7.3 Hz), 7.18 (1H, dd, J = 8.3, 7.8 Hz), 6.94 (1H, d, J = 2.3 Hz), 6.87 (1H, d, J = 2.3 Hz), 5.24-5.18 (1H, m), 5.08 (1H, m), 4.20 (2H, brs), 4.11-4.07 (2H, m), 3.97 (2H, m), 3.56-3.50 (4H, m), 3.30 (1H, brs), 3.26 (2H, brs), 3.04- 2.75 (3H, m), 2.66 (3H, brs), 2.35-2.24 (5H, m), 1.86-1.83 (1H, m), 1.66-1.60 (2H, m), 0.84 (2H, m), 0.53 (2H, m). | 634 |
| 4 | 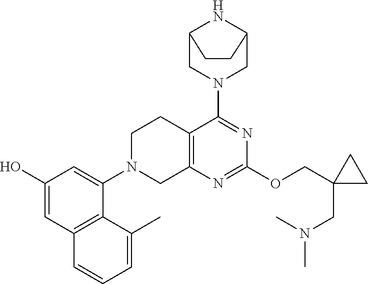 [SMILES: Cc1cccc2cc(O)cc(N3CCc4c(nc(OCC5(CN(C)C)CC5)nc4N4CC5CCC(C4)N5)C3)c12] | 1H NMR (400 MHz, DMSO-d6) δ = 9.62 (s, 1H), 7.49 (d, J = 8.1 Hz, 1H), 7.21 (dd, J = 7.1, 8.1 Hz, 1H), 6.99 (d, J = 6.9 Hz, 1H), 6.90 (d, J = 2.4 Hz, 1H), 6.83 (d, J = 2.4 Hz, 1H), 4.06-3.89 (m, 4H), 3.64-3.40 (m, 6H), 3.18- 3.13 (m, 1H), 3.03-2.97 (m, 2H), 2.92-2.84 (m, 1H), 2.77 (s, 3H), 2.18 (d, J = 2.6 Hz, 2H), 2.13 (s, 6H), 1.90-1.79 (m, 1H), 1,69-1.54 (m, 3H), 0.61-0.49 (m, 2H), 0.40-0.30 (m, 2H) | 529 |
| 5 | 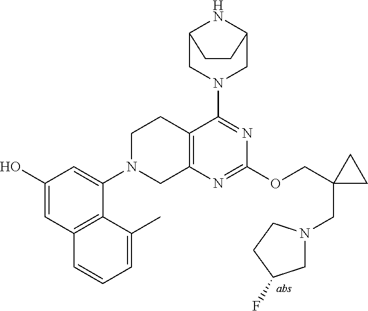 [SMILES: Cc1cccc2cc(O)cc(N3CCc4c(nc(OCC5(CN6CC[C@@H](F)C6)CC5)nc4N4CC5CCC(C4)N5)C3)c12] | 1H NMR (500 MHz, DMSO-d6) δ = 9.62 (s, 1H), 7.49 (d, J = 8.2 Hz, 1H), 7.21 (t, J = 7.7 Hz, 1H), 6.99 (d, J = 6.9 Hz, 1H), 6.90 (d, J = 2.4 Hz, 1H), 6.83 (d, J = 2.4 Hz, 1H), 5.23-5.08 (m, 1H), 4.12-3.89 (m, 4H), 3.62-3.40 (m, 8H), 3.19-3.13 (m, 1H), 3.02- 2.98 (m, 2H), 2.90-2.73 (m, 6H), 2.34-2.24 (m, 2H), 2.17- 1.99 (m, 1H), 1.93-1.75 (m, 2H), 1.71-1.54 (m, 3H), 0.57-0.51 (m, 2H), 0.40-0.36 (m, 2H) | 573 |
| 6 | 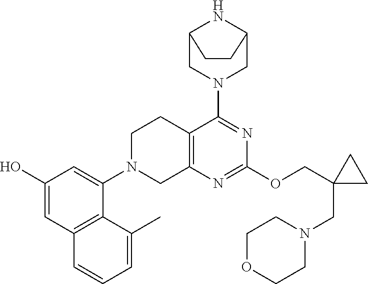 [SMILES: Cc1cccc2cc(O)cc(N3CCc4c(nc(OCC5(CN6CCOCC6)CC5)nc4N4CC5CCC(C4)N5)C3)c12] | 1H NMR (400 MHz, DMSO-d6) δ = 9.63 (s, 1H), 7.49 (d, J = 8.1 Hz, 1H), 7.20 (t, J = 7.6 Hz, 1H), 6.99 (d, J = 6.9 Hz, 1H), 6.90 (d, J = 2.4 Hz, 1H), 6.83 (d, J = 2.5 Hz, 1H), 4.13-3.88 (m, 4H), 3.61-3.40 (m, 10H), 3.16 (br d, J = 11.5 Hz, 1H), 3.01 (br d, J = 8.0 Hz, 2H), 2.87 (br d, J = 11.4 Hz, 1H), 2.77 (s, 3H), 2.35 (br d, J = 7.0 Hz, 4H), 2.25 (s, 2H), 1.89-1.77 (m, 1H), 1.71-1.55 (m, 3H), 0.61-0.50 (m, 2H), 0.39- 0.33 (m, 2H) | 571 |
| 7 | 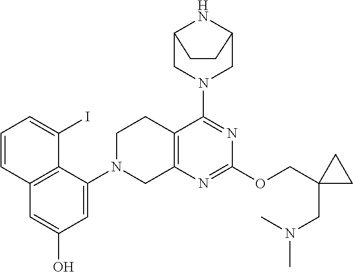 [SMILES: CN(C)CC1(COc2nc3c(c(N4CC5CCC(C4)N5)n2)CCN(c2cc(O)cc4cccc(I)c24)C3)CC1] | 1H NMR (400 MHz, DMSO-d6) δ = 9.91 (s, 1H), 7.94 (dd, J = 1.1, 7.3 Hz, 1H), 7.72 (dd, J = 1.1, 8.4 Hz, 1H), 7.01 (dd, J = 7.3, 8.1 Hz, 1H), 6.97-6.91 (m, 2H), 4.08-3.91 (m, 4H), 3.58-3.44 (m, 6H), 3.20-3.04 (m, 3H), 2.94- 2.84 (m, 1H), 2.23-2.16 (m, 2H), 2.16-2.12 (m, 6H), 1.88- 1.58 (m, 4H), 0.59-0.52 (m, 2H), 0.38-0.33 (m, 2H) | 641 |
| 8 | 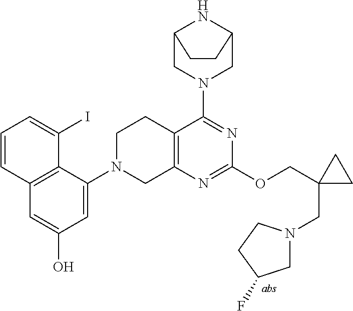 [SMILES: Oc1cc(N2CCc3c(nc(OCC4(CN5CC[C@@H](F)C5)CC4)nc3N3CC4CCC(C3)N4)C2)c2c(I)cccc2c1] | 1H NMR (400 MHz, DMSO-d6) δ = 9.90 (s, 1H), 7.94 (dd, J = 1.1, 7.2 Hz, 1H), 7.71 (dd, J = 0.9, 8.3 Hz, 1H), 7.00 (dd, J = 7.4, 8.1 Hz, 1H), 6.97-6.90 (m, 2H), 5.26-5.06 (m, 1H), 4.11-3.92 (m, 4H), 3.56-3.39 (m, 6H), 3.25- 3.06 (m, 6H), 2.98-2.73 (m, 4H), 2.20-1.99 (m, 1H), 1.92- 1.73 (m, 2H), 1.71-1.56 (m, 3H), 0.58-0.51 (m, 2H), 0.42-0.35 (m, 2H) | 685 |
| 9 | 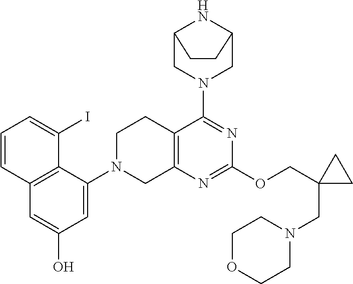 [SMILES: Oc1cc(N2CCc3c(nc(OCC4(CN5CCOCC5)CC4)nc3N3CC4CCC(C3)N4)C2)c2c(I)cccc2c1] | ¹H NMR (400 MHz, DMSO-d₆) δ ppm 9.92 (br s, 1 H), 7.94 (dd, J = 7.25, 1.13 Hz, 1 H), 7.71 (dd, J = 8.25, 0.88 Hz, 1 H), 7.00 (dd, J = 8.00, 7.38 Hz, 1 H), 6.90-6.97 (m, 2 H), 4.04-4.12 (m, 2 H), 4.01 (d, J = 17.39 Hz, 1 H), 3.95 (br d, J = 10.88 Hz, 1 H), 3.40- 3.57 (m, 8 H), 3.02-3.19 (m, 3 H), 2.88 (br d, J = 11.51 Hz, 1 H), 2.41-2.48 (m, 1 H), 2.36 (br s, 3 H), 2.26 (s, 2 H), 1.74-1.93 (m, 1 H), 1.58-1.70 (m, 3 H), 0.50-0.62 (m, 2 H), 0.31-0.41 (m, 2 H) | 683 |
| 10 | 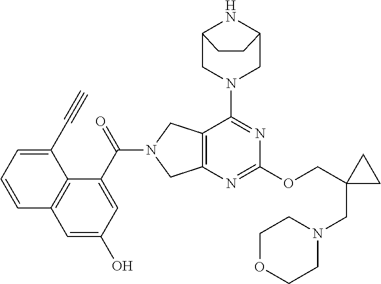 [SMILES: C#Cc1cccc2cc(O)cc(C(=O)N3Cc4nc(OCC5(CN6CCOCC6)CC5)nc(N5CC6CCC(C5)N6)c4C3)c12] | 1H NMR (400 MHz, DMSO-d6) δ = 10.22 (br s, 1H), 7.90-7.84 (m, 1H), 7.58-7.53 (m, 1H), 7.48- 7.41 (m, 1H), 7.32-7.26 (m, 1H), 7.10-7.05 (m, 1H), 4.97-4.61 (m, 2H), 4.54-4.39 (m, 2H), 4.18- 3.90 (m, 5H), 3.51 (br d, J = 4.3 Hz, 6H), 3.21-3.10 (m, 2H), 2.94-2.81 (m, 1H), 2.42-2.20 (m, 6H), 1.75-1.42 (m, 4H), 0.62- 0.48 (m, 2H), 0.41-0.30 (m, 2H) | 595 |
| 11 | 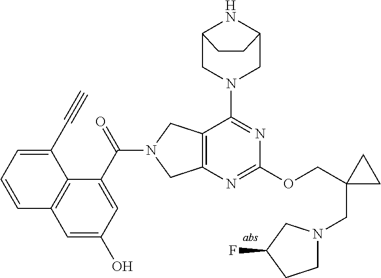 [SMILES: C#Cc1cccc2cc(O)cc(C(=O)N3Cc4nc(OCC5(CN6CC[C@@H](F)C6)CC5)nc(N5CC6CCC(C5)N6)c4C3)c12] | ¹H NMR (400 MHz, DMSO-d₆) δ = 7.87-7.82 (m, 1H), 7.56-7.50 (m, 1H), 7.46-7.39 (m, 1H), 7.09- 7.05 (m, 1H), 5.28-5.04 (m, 1H), 4.97-4.60 (m, 2H), 4.54- 4.39 (m, 2H), 4.18-3.91 (m, 5H), 3.50 (br s, 2H), 3.21-3.08 (m, 1H), 2.92-2.69 (m, 3H), 2.48- 2.24 (m, 3H), 2.19-1.71 (m, 2H), 1.71-1.61 (m, 3H), 1.56-1.29 (m, 2H), 0.60-0.47 (m, 2H), 0.44- 0.32 (m, 2H). | 597 |
| 12 | 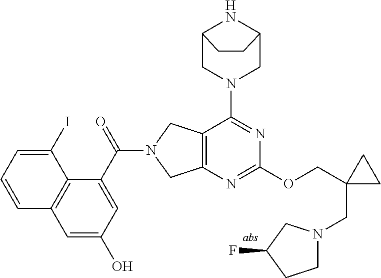 [SMILES: O=C(c1cc(O)cc2cccc(I)c12)N1Cc2nc(OCC3(CN4CC[C@@H](F)C4)CC3)nc(N3CC4CCC(C3)N4)c2C1] | ¹H NMR (400 MHz, DMSO-d₆) δ = 10.43-10.10 (m, 1H), 8.03-7.97 (m, 1H), 7.87-7.81 (m, 1H), 7.29- 7.25 (m, 1H), 7.17-7.10 (m, 2H), 5.27-5.04 (m, 1H), 5.03- 4.70 (m, 2H), 4.66-4.51 (m, 1H), 4.18-3.94 (m, 5H), 3.75-3.62 (m, 1H), 3.51 (br s, 2H), 3.18- 3.08 (m, 2H), 2.98-2.62 (m, 4H), 2.43-2.22 (m, 3H), 2.14-1.75 (m, 2H), 1.70-1.43 (m, 4H), 0.59- 0.49 (m, 2H), 0.44-0.34 (m, 2H ). | 699 |
| 13 | 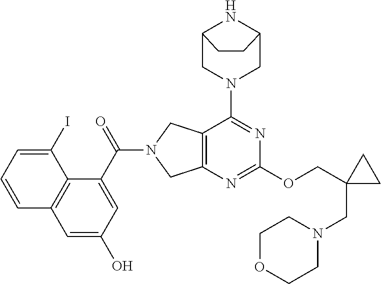 [SMILES: O=C(c1cc(O)cc2cccc(I)c12)N1Cc2nc(OCC3(CN4CCOCC4)CC3)nc(N3CC4CCC(C3)N4)c2C1] | ¹H NMR (400 MHz, DMSO-d₆) δ = 10.65-9.82 (br s, 1H), 8.03- 7.97 (m, 1H), 7.88-7.82 (m, 1H), 7.31-7.25 (m, 1H), 7.18-7.09 (m, 2H), 5.06-4.45 (m, 3H), 4.19- 3.94 (m, 5H), 3.75-3.49 (m, 6H), 3.14 (br d, J = 11.9 Hz, 1H), 2.97-2.86 (m, 1H), 2.45-2.18 (m, 6H), 1.74-1.44 (m, 4H), 0.62- 0.48 (m, 2H), 0.42-0.30 (m, 2H) | 697 |
| 14 | 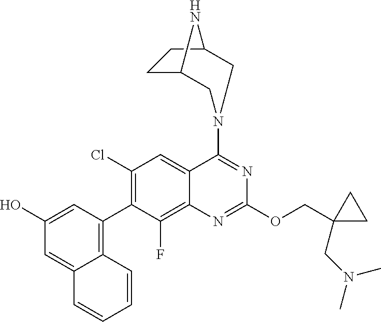 [SMILES: CN(C)CC1(COc2nc(N3CC4CCC(C3)N4)c3cc(Cl)c(-c4cc(O)cc5ccccc45)c(F)c3n2)CC1] | 1H NMR (400 MHz, DMSO-d6) δ ppm 0.36-0.45 (m, 2 H) 0.55-0.65 (m, 2 H) 1.77 (br s, 3 H) 2.17- 2.23 (m, 3 H) 2.27-2.34 (m, 1 H) 2.45 (br s, 6 H) 3.57-3.62 (m, 2 H) 4.18 (s, 2 H) 4.31-4.42 (m, 2 H) 7.00 (br d, J = 2.22 Hz, 1 H) 7.11-7.31 (m, 3 H) 7.35- 7.43 (m, 1 H) 7.75 (s, 1 H) 7.87- 7.93 (m, 1 H) 8.10 (s, 1 H) 8.26 (s, 1 H) | 562 |
| 15 | 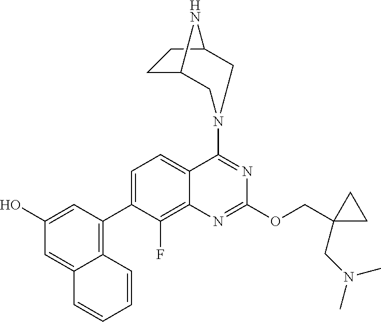 [SMILES: CN(C)CC1(COc2nc(N3CC4CCC(C3)N4)c3ccc(-c4cc(O)cc5ccccc45)c(F)c3n2)CC1] | 1H NMR (400 MHz, DMSO-d6) δ ppm 0.52-0.64 (m, 2 H) 0.68-0.79 (m, 2 H) 1.82-2.03 (m, 4 H) 2.23- 2.29 (m, 1 H) 2.45 (s, 6 H) 2.53 (m, 2 H) 2.60-2.65 (m, 1 H) 3.58- 3.70 (m, 2 H) 4.14-4.28 (m, 2 H) 4.30-4.48 (m, 2 H) 7.04 (m, 1 H) 7.22 (m, 1 H) 7.36-7.45 (m, 4 H) 7.72-7.81 (m, 1 H) 7.82- 7.90 (m, 1 H) 8.06-8.12 (m, 1 H) 9.95-10.07 (m, 1 H) | 528 |
| 16 | 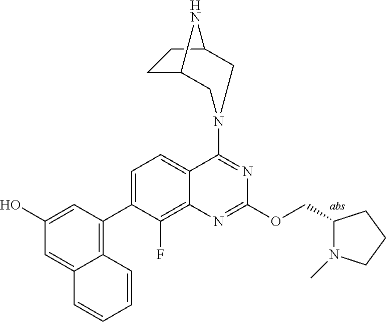 [SMILES: CN1CCC[C@H]1COc1nc(N2CC3CCC(C2)N3)c2ccc(-c3cc(O)cc4ccccc34)c(F)c2n1] | 1H NMR (400 MHz, DMSO-d6) δ ppm 1.92 (m, 7 H) 2.07-2.22 (m, 1 H) 2.23-2.36 (m, 1 H) 2.57- 2.64 (m, 1 H) 2.69-2.77 (s, 3 H) 2.85-3.01 (m, 3 H) 3.65- 3.77 (m, 2 H) 4.03-4.18 (m, 2 H) 4.34-4.49 (m, 2 H) 7.04 (d, J = 1.90 Hz, 1 H) 7.23 (br s, 2 H) 7.27-7.37 (m, 2 H) 7.37-7.43 (m, 1 H) 7.72-7.80 (m, 1 H) 7.85- 7.92 (m, 1 H) | 514 |
| 17 | 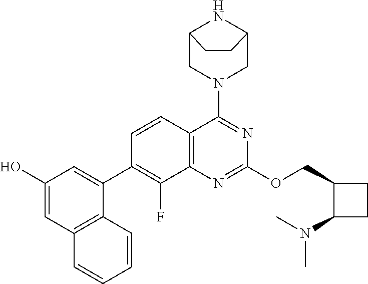 [SMILES: CN(C)[C@@H]1CC[C@@H]1COc1nc(N2CC3CCC(C2)N3)c2ccc(-c3cc(O)cc4ccccc34)c(F)c2n1] | 1H NMR (500 MHz, DMSO-d6) δ ppm 1.51-1.62 (m, 1 H) 1.61-1.76 (m, 4 H) 1.78-1.85 (m, 1 H) 1.93- 2.00 (m, 2 H) 2.03 (s, 6 H) 2.37 (s, 1 H) 2.64 (s, 1 H) 2.69 (m, 1 H) 3.36 (m, 3 H) 4.21-4.28 (m, 1 H) 4.28-4.37 (m, 1 H) 4.37- 4.47 (m, 1 H) 4.61-4.73 (m, 1 H) 7.10 (d, J = 2.44 Hz, 1 H) 7.20- 7.29 (m, 3 H) 7.44 (br d, J = 8.24 Hz, 2 H) 7.81 (d, J = 8.24 Hz, 1 H) 7.86 (d, J = 8.39 Hz, 1 H) 8.32 (s, 1 H) 9.97 (s, 1 H) | 528 |
| 18 | 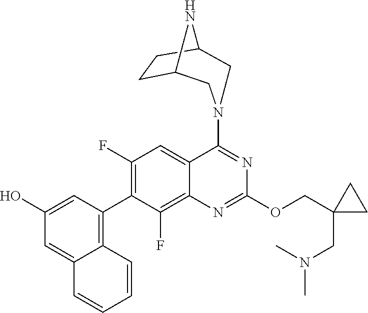 [SMILES: CN(C)CC1(COc2nc(N3CC4CCC(C3)N4)c3cc(F)c(-c4cc(O)cc5ccccc45)c(F)c3n2)CC1] | 1H NMR (500 MHz, DMSO-d6) δ ppm 0.40 (d, J = 1.22 Hz, 2 H) 0.63 (d, J = 1.37 Hz, 2 H) 1.61-1.72 (m, 4 H) 2.16 (s, 6 H) 2.23 (s, 2 H) 2.35-2.39 (m, 1 H) 2.63-2.67 (m, 1 H) 3.47 (br d, J = 14.80 Hz, 2 H) 4.18-4.24 (m, 2 H) 4.26- 4.33 (m, 2 H) 7.15 (d, J = 2.29 Hz, 1 H) 7.24-7.28 (m, 1 H) 7.30- 7.32 (m, 1 H) 7.34 (s, 1 H) 7.44- 7.49 (m, 1 H) 7.68-7.73 (m, 1 H) 7.80-7.84 (m, 1 H) 8.32 (s, 1 H) 10.04 (s, 1 H) | 546 |
| 19 | 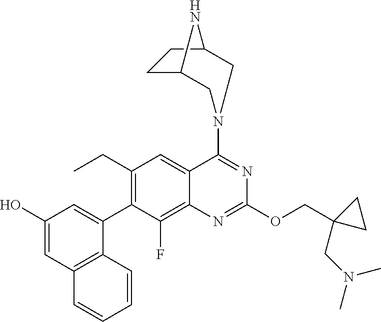 [SMILES: CCc1cc2c(N3CC4CCC(C3)N4)nc(OCC3(CN(C)C)CC3)nc2c(F)c1-c1cc(O)cc2ccccc12] | 1H NMR (500 MHz, DMSO-d6) δ ppm 0.37-0.42 (m, 2 H) 0.60-0.65 (m, 2 H) 0.95 (t, J = 7.48 Hz, 3 H) 1.71 (s, 4 H) 2.16 (s, 6 H) 2.21- 2.24 (m, 2 H) 2.29 (s, 2 H) 2.37- 2.38 (m, 1 H) 2.63-2.68 (m, 1 H) 2.64 (dt, J = 3.78, 1.85 Hz, 1 H) 3.44-3.50 (m, 2 H) 4.18- 4.21 (m, 2 H) 4.25-4.34 (m, 2 H) 7.02 (d, J = 2.44 Hz, 1 H) 7.12- 7.16 (m, 1 H) 7.17-7.22 (m, 1 H) 7.24-7.26 (m, 1 H) 7.38- 7.49 (m, 1 H) 7.64-7.75 (m, 1 H) 7.77-7.86 (m, 1 H) 9.96 (s, 1 H) | 556 |
| 20 | 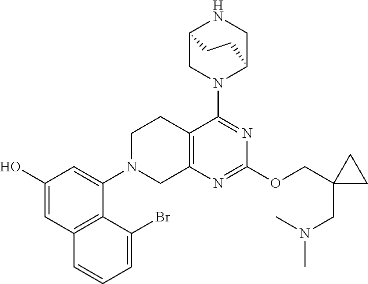 [SMILES: CN(C)CC1(COc2nc3c(c(N4C[C@@H]5CC[C@H]4CN5)n2)CCN(c2cc(O)cc4cccc(Br)c24)C3)CC1] | 1H NMR (400 MHz, CHLOROFORM-d) δ = 7.53-7.45 (m, 2H), 7.10-7.02 (m, 1H), 6.85-6.75 (m, 2H), 4.42- 3.95 (m, 5H), 3.81-2.78 (m, 10H), 2.52-1.68 (m, 11H), 0.71- 0.62 (m, 2H), 0.52-0.40 (m, 2H) | 593 |
| 21 | 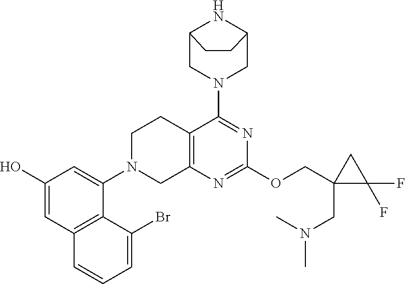 [SMILES: CN(C)CC1(COc2nc3c(c(N4CC5CCC(C4)N5)n2)CCN(c2cc(O)cc4cccc(Br)c24)C3)CC1(F)F] | 1H NMR (400 MHz, CHLOROFORM-d) δ = 7.62-7.55 (m, 2H), 7.17-7.11 (m, 1H), 6.90 (d, J = 2.4 Hz, 1H), 6.86-6.82 (m, 1H), 4.63-3.99 (m, 4H), 3.73-2.93 (m, 11H), 2.52-1.17 (m, 11H), 1.00-0.76 (m, 2H) | 629 |
| 22 | 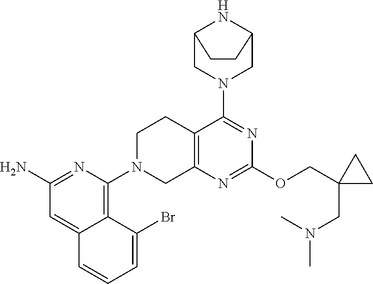 [SMILES: CN(C)CC1(COc2nc3c(c(N4CC5CCC(C4)N5)n2)CCN(c2nc(N)cc4cccc(Br)c24)C3)CC1] | 1H NMR (400 MHz, DMSO-d6) δ = 7.45-7.40 (m, 1H), 7.34-7.30 (m, 1H), 7.21-7.14 (m, 1H), 6.24- 6.21 (m, 1H), 5.94-5.85 (m, 2H), 4.34-4.17 (m, 2H), 4.05- 4.00 (m, 2H), 3.82-3.57 (m, 4H), 3.06-2.88 (m, 4H), 2.15 (s, 8H), 1.77-1.60 (m, 4H), 1.28-1.21 (m, 2H), 0.60-0.53 (m, 2H), 0.40- 0.33 (m, 2H) | 593 |
| 23 | 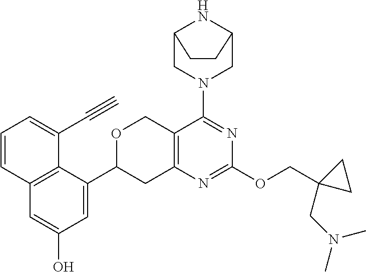 [SMILES: C#Cc1cccc2cc(O)cc(C3Cc4nc(OCC5(CN(C)C)CC5)nc(N5CC6CCC(C5)N6)c4CO3)c12] | 1H-NMR (400 MHz, DMSO-D6) δ: 9.95 (1H, br s), 7.85-7.80 (1H, m), 7.59-7.55 (1H, m), 7.47-7.43 (1H, m), 7.41-7.35 (1H, m), 7.17- 7.14 (1H, m), 6.61-6.54 (1H, m), 4.98 (1H, d, J = 13.9 Hz), 4.70 (1H, d, J = 13.9 Hz), 4.52 (1H, s), 4.05-4.02 (2H, m), 3.92-3.84 (1H, m), 3.46-3.20 (6H, m), 2.93- 2.86 (1H, m), 2.62-2.54 (1H, m), 2.19 (2H, br s), 2.14 (6H, s), 1.88-1.51 (4H, m), 0.62-0.53 (2H, m), 0.40-0.31 (2H, m). | 540 |
| 24 | 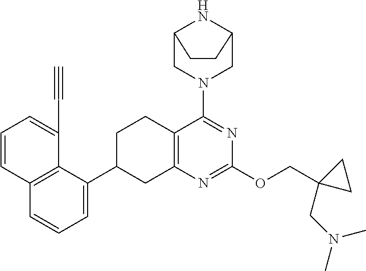 [SMILES: C#Cc1cccc2cccc(C3CCc4c(nc(OCC5(CN(C)C)CC5)nc4N4CC5CCC(C4)N5)C3)c12] | 1H-NMR (400 MHz, CDCl3) δ: 7.93-7.88 (1H, m), 7.88-7.84 (1H, m), 7.79-7.73 (1H, m), 7.50-7.46 (2H, m), 7.45-7.39 (1H, m), 5.34- 5.25 (1H, m), 4.19 (2H, br s), 3.96-3.88 (1H, m), 3.61-3.53 (3H, m), 3.45-3.37 (1H, m), 3.39 (2H, s), 3.31-3.24 (1H, m), 3.01-2.87 (2H, m), 2.83-2.71 (1H, m), 2.58- 2.50 (1H, m), 2.39 (1H, d, J = 12.5 Hz), 2.35 (1H, d, J = 12.5 Hz), 2.27 (6H, s), 2.08-1.61 (5H, m), 0.69-0.63 (2H, m), 0.48-0.40 (2H, m). | 522 |
| 25 | 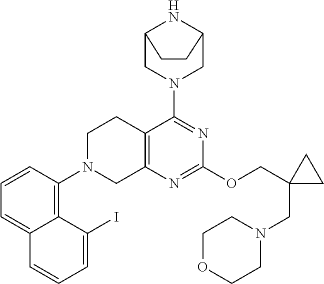 [SMILES: Ic1cccc2cccc(N3CCc4c(nc(OCC5(CN6CCOCC6)CC5)nc4N4CC5CCC(C4)N5)C3)c12] | 1H NMR (CHLOROFORM-d, 400 MHz) δ 8.24 (dd, 1H, J = 1.1, 7.3 Hz), 7.84 (dd, 1H, J = 0.9, 8.2 Hz), 7.63 (dd, 1H, J = 0.9, 8.2 Hz), 7.48 (t, 1H, J = 7.8 Hz), 7.30 (d, 1H, J = 7.0 Hz), 7.08 (dd, 1H, J = 7.4, 8.0 Hz), 4.33 (d, 1H, J = 17.9 Hz), 4.2-4.3 (m, 2H), 4.01 (br d, 1H, J = 11.4 Hz), 3.6-3.8 (m, 7H), 3.4-3.5 (m, 1H), 3.31 (br d, 1H, J = 12.3 Hz), 3.1-3.2 (m, 2H), 3.07 (br d, 1H, J = 12.3 Hz), 2.4-2.6 (m, 6H), 2.0-2.1 (m, 1H), 1.7-1.9 (m, 3H), 1.2-1.4 (m, 2H), 0.6-0.7 (m, 2H), 0.4-0.5 (m, 2H) | 667 |
| 26 | 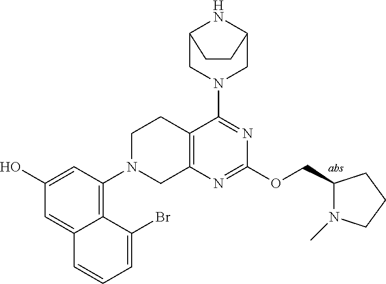 [SMILES: CN1CCC[C@@H]1COc1nc2c(c(N3CC4CCC(C3)N4)n1)CCN(c1cc(O)cc3cccc(Br)c13)C2] | 1H NMR (DMSO-d6, 500 MHz) δ 9.94 (s, 1H), 7.70 (dd, 1H, J = 0.9, 8.4 Hz), 7.54 (dd, 1H, J = 1.1, 7.4 Hz), 7.20 (t, 1H, J = 7.8 Hz), 6.96 (d, 1H, J = 2.3 Hz), 6.89 (d, 1H, J = 2.3 Hz), 4.2-4.2 (m, 1H), 4.11 (d, 1H, J = 17.4 Hz), 4.0-4.0 (m, 2H), 3.56 (br d, 2H, J = 16.6 Hz), 3.3-3.3 (m, 4H), 3.17 (br d, 1H, J = 12.2 Hz), 3.06 (br d, 2H, J = 7.9 Hz), 2.9-3.0 (m, 2H), 2.32 (d, 3H, J = 0.9 Hz), 2.15 (d, 1H, J = 8.4 Hz), 1.8-1.9 (m, 2H), 1.6-1.7 (m, 7H) | 579 |
| 27 | 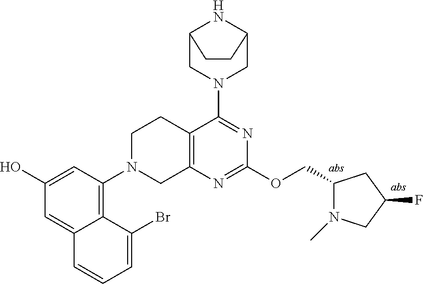 [SMILES: CN1C[C@H](F)C[C@H]1COc1nc2c(c(N3CC4CCC(C3)N4)n1)CCN(c1cc(O)cc3cccc(Br)c13)C2] | 1H NMR (CHLOROFORM-d, 400 MHz) δ 7.5-7.5 (m, 2H), 7.07 (t, 1H, J = 7.8 Hz), 6.7-6.9 (m, 2H), 5.0- 5.2 (m, 1H), 4.2-4.4 (m, 3H), 4.06 (br d, 1H, J = 12.1 Hz), 3.5- 3.7 (m, 4H), 3.2-3.4 (m, 1H), 3.0-3.1 (m, 2H), 2.6-2.7 (m, 1H), 2.52 (d, 3H, J = 1.1 Hz), 2.2-2.5 (m, 1H), 1.7-2.0 (m, 4H), 1.2-1.4 (m, 3H), 0.8-1.0 (m, 3H) | 597 |
| 28 | 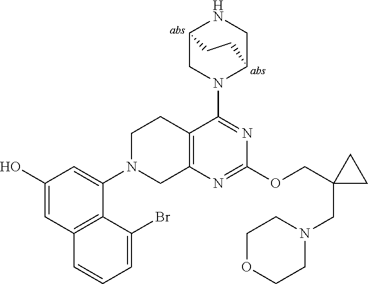 [SMILES: Oc1cc(N2CCc3c(nc(OCC4(CN5CCOCC5)CC4)nc3N3C[C@@H]4CC[C@H]3CN4)C2)c2c(Br)cccc2c1] | 1H NMR (400 MHz, CHLOROFORM-d) δ = 7.59-7.49 (m, 2H), 7.13-7.05 (m, 1H), 6.93-6.75 (m, 2H), 4.51- 3.97 (m, 5H), 3.83-2.90 (m, 15H), 2.61-1.69 (m, 8H), 0.73- 0.53 (m, 2H), 0.49-0.33 (m, 2H) | 635 |
| 29 | 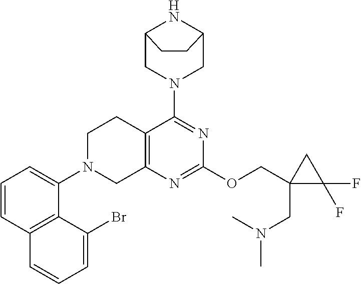 [SMILES: CN(C)CC1(COc2nc3c(c(N4CC5CCC(C4)N5)n2)CCN(c2cccc4cccc(Br)c24)C3)CC1(F)F] | 1H NMR (400 MHz, CHLOROFORM-d) δ = 7.84-7.78 (m, 2H), 7.63 (d, J = 8.0 Hz, 1H), 7.46 (dt, J = 1.5, 7.8 Hz, 1H), 7.28-7.22 (m, 2H), 4.63-4.53 (m, 1H), 4.40 (d, J = 17.8 Hz, 1H), 4.34-4.23 (m, 1H), 4.12-4.00 (m, 1H), 3.79 (dd, J = 3.6, 17.8 Hz, 1H), 3.72-3.48 (m, 4H), 3.32 (d, J = 12.4 Hz, 1H), 3.23-2.93 (m, 4H), 2.59- 2.48 (m, 1H), 2.26 (s, 6H), 2.23- 2.16 (m, 1H), 2.08-1.95 (m, 1H), 1.95-1.50 (m, 4H), 1.31- 1.15 (m, 2H) | 613 |
| 30 | 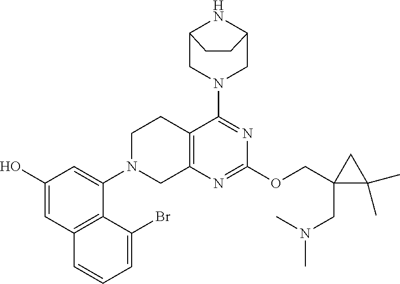 [SMILES: CN(C)CC1(COc2nc3c(c(N4CC5CCC(C4)N5)n2)CCN(c2cc(O)cc4cccc(Br)c24)C3)CC1(C)C] | 1H NMR (400 MHz, DMSO-d6) δ = 9.95 (br s, 1H), 7.70 (dd, J = 0.8, 8.4 Hz, 1H), 7.54 (dd, J = 0.8, 7.4 Hz, 1H), 7.25-7.15 (m, 1H), 6.96 (d, J = 2.3 Hz, 1H), 6.89 (t, J = 2.3 Hz, 1H), 4.52- 4.37 (m, 1H), 4.20-4.05 (m, 1H), 4.01-3.85 (m, 2H), 3.64-3.00 (m, 12H), 2.95-2.82 (m, 1H), 2.61 (dd, J = 3.1, 12.3 Hz, 1H), 2.12 (s, 6H), 2.07 (dd, J = 3.4, 12.4 Hz, 1H), 1.83 (br d, J = 6.4 Hz, 1H), 1.12 (s, 3H), 1.11 (s, 3H), 0.56 (t, J = 3.7 Hz, 1H), 0.42-0.33 (m, 1H) | 621 |
| 31 | 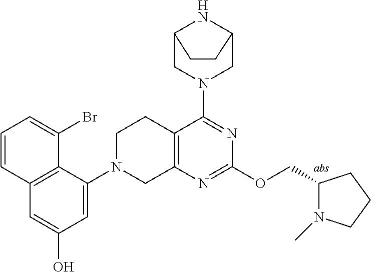 [SMILES: CN1CCC[C@H]1COc1nc2c(c(N3CC4CCC(C3)N4)n1)CCN(c1cc(O)cc3cccc(Br)c13)C2] | 1H NMR (400 MHz, DMSO-d6) δ = 7.72-7.63 (m, 1H), 7.54-7.47 (m, 1H), 7.22-7.14 (m, 1H), 6.98- 6.77 (m, 2H), 4.24-4.17 (m, 1H), 4.14-4.05 (m, 1H), 4.02- 3.94 (m, 1H), 3.59-3.47 (m, 2H), 3.44-3.35 (m, 5H), 3.18-2.86 (m, 5H), 2.23-2.08 (m, 1H), 1.96- 1.79 (m, 2H), 1.69-1.53 (m, 6H), 1.37-1.16 (m, 4H), 0.88- 0.80 (m, 1H) | 579 |
| 32 | 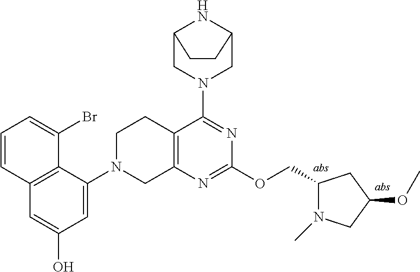 [SMILES: CO[C@@H]1C[C@@H](COc2nc3c(c(N4CC5CCC(C4)N5)n2)CCN(c2cc(O)cc4cccc(Br)c24)C3)N(C)C1] | 1H NMR (400 MHz, DMSO-d6) δ = 9.93 (br s, 1H), 7.69 (d, J = 7.8 Hz, 1H), 7.53 (d, J = 6.6 Hz, 1H), 7.19 (t, J = 7.8 Hz, 1H), 6.95 (d, J = 2.0 Hz, 1H), 6.88 (d, J = 2.4 Hz, 1H), 4.24-4.18 (m, 1H), 4.13-3.95 (m, 3H), 3.87-3.80 (m, 1H), 3.55 (br d, J = 15.5 Hz, 2H), 3.45-3.35 (m, 5H), 3.30 (s, 3H), 3.20-3.03 (m, 6H), 2.89 (br d, J = 11.8 Hz, 1H), 2.61- 2.52 (m, 2H), 2.14 (dd, J = 5.9, 9.6 Hz, 1H), 1.89-1.74 (m, 3H), 1.68-1.54 (m, 3H) | 609 |
| 33 | 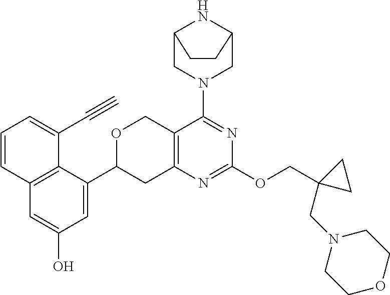 [SMILES: C#Cc1cccc2cc(O)cc(C3Cc4nc(OCC5(CN6CCOCC6)CC5)nc(N5CC6CCC(C5)N6)c4CO3)c12] | 1H-NMR (400 MHz, DMSO-D6) δ: 9.95 (1H, s), 7.86-7.80 (1H, m), 7.60-7.54 (1H, m), 7.47-7.42 (1H, m), 7.42-7.35 (1H, m), 7.18-7.14 (1H, m), 6.62-6.54 (1H, m), 4.98 (1H, d, J = 14.0 Hz), 4.70 (1H, d, J = 14.0 Hz), 4.50 (1H, s), 4.13 (1H, d, J = 10.8 Hz), 4.07 (1H, d, J = 10.8 Hz), 3.93-3.85 (1H, m), 3.57-3.49 (4H, m), 3.46- 3.19 (6H, m), 2.93-2.85 (1H, m), 2.62-2.47 (1H, m), 2.42-2.31 (4H, m), 2.30-2.23 (2H, m), 1.89-1.50 (4H, m), 0.63-0.53 (2H, m), 0.41- 0.31 (2H, m). | 582 |
| 34 | 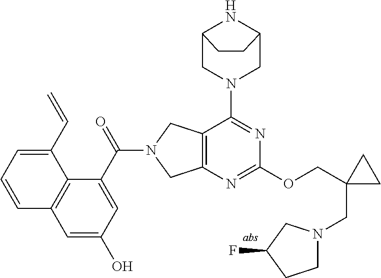 [SMILES: C=Cc1cccc2cc(O)cc(C(=O)N3Cc4nc(OCC5(CN6CC[C@@H](F)C6)CC5)nc(N5CC6CCC(C5)N6)c4C3)c12] | 1H NMR (400 MHz, DMSO-d6) δ ppm 7.79-7.73 (m, 1 H) 7.48-7.41 (m, 1 H) 7.33-7.24 (m, 2 H) 7.18- 7.01 (m, 2 H) 5.48-5.35 (m, 1 H) 5.22-4.79 (m, 3 H) 4.59- 4.44 (m, 1 H) 4.20-3.81 (m, 5 H) 3.72-3.52 (m, 3 H) 3.17- 3.11 (m, 1 H) 2.89-2.66 (m, 3 H) 2.46-2.20 (m, 3 H) 2.14- 1.74 (m, 2 H) 1.72-1.59 (m, 3 H) 1.55-1.27 (m, 2 H) 0.59- 0.45 (m, 2 H) 0.43-0.33 (m, 2 H) | 599 |
| 35 | 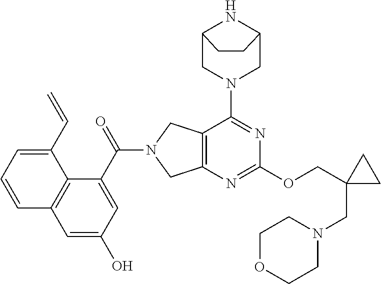 [SMILES: C=Cc1cccc2cc(O)cc(C(=O)N3Cc4nc(OCC5(CN6CCOCC6)CC5)nc(N5CC6CCC(C5)N6)c4C3)c12] | 1H NMR (400 MHz, DMSO-d6) δ ppm 7.80-7.73 (m, 1 H) 7.48-7.42 (m, 1 H) 7.34-7.24 (m, 2 H) 7.18- 7.00 (m, 2 H) 5.49-5.35 (m, 1 H) 5.24-5.09 (m, 1 H) 5.01- 4.83 (m, 1 H) 4.61-4.46 (m, 1 H) 4.19-3.99 (m, 4 H) 3.89- 3.82 (m, 1 H) 3.70-3.55 (m, 6 H) 3.21-3.16 (m, 2 H) 2.94- 2.82 (m, 1 H) 2.39-2.18 (m, 6 H) 1.77-1.45 (m, 4 H) 0.61- 0.47 (m, 2 H) 0.42-0.29 (m, 2 H) | 597 |
| 36 | 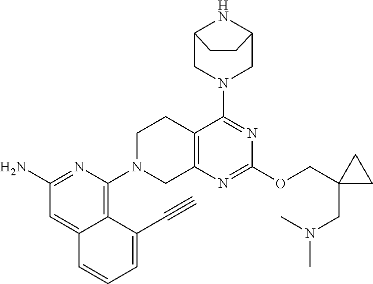 [SMILES: C#Cc1cccc2cc(N)nc(N3CCc4c(nc(OCC5(CN(C)C)CC5)nc4N4CC5CCC(C4)N5)C3)c12] | 1H NMR (400 MHz, DMSO-d6) δ = 7.49-7.44 (m, 1H), 7.36-7.29 (m, 1H), 7.28-7.23 (m, 1H), 6.26- 6.23 (m, 1H), 5.84-5.77 (m, 2H), 4.40-3.99 (m, 5H), 3.83- 3.72 (m, 1H), 3.07-2.94 (m, 3H), 2.15 (s, 9H), 1.86-1.56 (m, 5H), 1.27-1.22 (m, 2H), 1.03-0.97 (m, 3H), 0.58-0.54 (m, 2H), 0.39- 0.32 (m, 2H) | 539 |
| 37 | 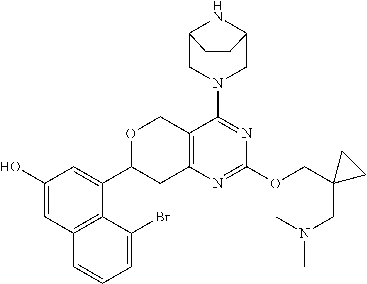 [SMILES: CN(C)CC1(COc2nc3c(c(N4CC5CCC(C4)N5)n2)COC(c2cc(O)cc4cccc(Br)c24)C3)CC1] | 1H-NMR (400 MHz, DMSO-D6) δ: 10.06 (1H, br s), 7.83-7.78 (1H, m), 7.71-7.65 (1H, m), 7.57-7.53 (1H, m), 7.29-7.22 (1H, m), 7.20- 7.16 (1H, m), 6.47-6.38 (1H, m), 5.02 (1H, d, J = 13.8 Hz), 4.70 (1H, d, J = 13.8 Hz), 4.05 (2H, s), 3.94-3.86 (1H, m), 3.48-3.20 (7H, m), 2.93-2.85 (1H, m), 2.58- 2.54 (2H, m), 2.14 (6H, s), 1.87- 1.76 (1H, m), 1.72-1.52 (3H, m), 0.61-0.52 (2H, m), 0.40-0.31 (2H, m). | 594 |

Test Example 1: Evaluation of inhibitory activity of compounds on KRAS G12D nucleotide (GDP-GTP) exchange reaction in vitro

Recombinant KRAS G12D (conjugated N-terminus His₆-tag, TEV protease cleavage site and KRAS G12D residues 1-169 (SEQ ID NO: 1), prepared by the method described as below) and cleaved recombinant SOS1 (residues 564-1049(SEQ ID NO: 2), prepared by the method as below) proteins were expressed in E. coli and purified by affinity chromatography.

To prepare the recombinant KRAS G12D, the codon-optimized DNA sequence for the recombinant KRAS G12D (conjugated N-terminus His₆-tag, TEV protease cleavage site and KRAS G12D residues 1-169 (SEQ ID NO: 3))was synthesized by GeneArt Technology (Life Technologies, Carlsbad, CA, US). The construct was subcloned into the expression vector pET28a and transformed into Escherichia coli BL21 (DE3) strains (Novagen, Madison, WI, US).

The transformed strain was cultivated in 2 L Luria Broth medium with 25 μg/mL kanamycin at a temperature of 37° C. to a density of 0.6 (OD600), then induced for expression with 500 mM IPTG and further cultivated for 4 h. The cell pellet was resuspended in ice-cold lysis buffer containing 50 mM Tris-HCl (pH 7.5), 200 mM NaCl, and 100 μM TCEP (tris(2-carboxyethyl)phosphine).

After sonication, the disrupted debris was removed by centrifugation. The supernatant was applied on to Ni-NTA affinity gels, and the recombinant human KRAS (G12D) eluted fraction was collected. The buffer of the collected fraction was exchanged to the buffer containing 50 mM Tris-HCl (pH 7.5), 200 mM NaCl, 10% Glycerol, and 5 mM DTT by PD-10 column (GE-Healthcare, Chicago, IL, US).

To prepare the cleaved recombinant SOS1, the codon optimized DNA sequences for the recombinant SOS1 (residues 564-1049 with a conjugated N-terminus His₆-tag and TEV protease cleavage site (SEQ ID NO: 4)) was synthesized by GeneArt Technology (Life Technologies). The construct was subcloned into the expression vector pET28a and transformed into Escherichia coli BL21 (DE3) strains (Novagen, Madison, WI, USA).

The transformed strain was cultivated in 2 L Terrific Broth medium with 25 μg/mL kanamycin at a temperature of 37° C. to a density of 0.8 (OD600), then shifted to a temperature of 16° C., induced for expression with 400 mM IPTG, and further cultivated for 16 h. The cell pellet was resuspended in ice-cold lysis buffer containing 50 mM Tris-HCl (pH 7.5), 200 mM NaCl, and 100 μM TCEP.

After sonication, the disrupted debris was removed by centrifugation. The supernatant was applied on to Ni-NTA affinity gels, and the recombinant human SOS1 eluted fraction was collected. Then, His₆-tagged TEV protease was added to the collected fraction, dialyzed with the ice-cold lysis buffer for 16 h at a temperature of 4° C., and applied on to Ni-NTA affinity gels. The flow-through fraction containing the tag-cleavaged recombinant human SOS1 was collected. The buffer of the collected fraction was exchanged to the buffer containing 50 mM Tris-HCl (pH 7.5), 200 mM NaCl, 10% Glycerol, and 5 mM DTT by PD-10 column (GE-Healthcare).

To prepare BODIPY FL (fluorescent dye) GDP-bound KRAS G12D protein, 50 μM KRAS G12D protein was incubated with 0.5 mM BODIPY FL GDP in a loading buffer (20 mM Tris-HCl (pH 7.5), 50 mM NaCl, 1 mM DTT and 2.5 mM EDTA) for 1 hour on ice. After the incubation, MgCl₂ was added to a final concentration of 10 mM, followed by incubation at room temperature for 30 minutes.

The mixture was allowed to pass through a NAP-5 column to remove free nucleotides and purified BODIPY FL GDP-bound KRAS G12D protein was used for compound evaluation.

For the measurement of the inhibitory activity of compounds on GDP-GTP exchange rate of recombinant KRAS G12D, BODIPY FL GDP-bound KRAS G12D protein was incubated with various concentrations of compound in a reaction buffer (20 mM Tris-HCl (pH 7.5), 100 mM NaCl, 1 mM MgCl2, 2 mM DTT, 0.1% Tween 20) at 25° C. for 1 hour.

After the incubation, recombinant SOS1 and GMPPNP (guanosine-5′-[((β, γ)-imido]triphosphate, tetralithium salt) (Jena Bioscience GmbH, Jena, Germany) were added and incubated at room temperature for 30 minutes to proceed SOS1-dependent GDP-GTP exchange reaction on KRAS G12D. Replacement of BODIPY FL GDP by GMPPNP was measured by calculating the ratio of fluorescence intensities of BODIPY FL before and after the exchange reaction.

Inhibition % was calculated with setting the fluorescence ratio from the reaction without test compound (DMSO control) and the fluorescence ratio from the reaction without SOS1 and GMPPNP as 0% and 100% inhibition, respectively. IC50 values were calculated from dose titration curve using curve fitting by XLfit software (IDBS, Boston, MA, US). The following table (Table 2) shows the inhibitory activity IC50 (nM) of the test compounds.

TABLE 2

| | Exchange |
|---|---|
| | G12D |
| Example | IC50(nM) |
| 1 | 0.8 |
| 2 | 1.5 |
| 3 | 1.2 |
| 4 | 1.5 |
| 5 | 6.2 |
| 6 | 3.2 |
| 7 | 0.7 |
| 8 | 0.9 |
| 9 | 0.7 |
| 10 | 1.5 |
| 11 | 1.4 |
| 12 | 4.4 |
| 13 | 5.5 |
| 14 | 8.9 |
| 15 | 4.7 |
| 16 | 10.2 |
| 17 | 14.6 |
| 18 | 5.1 |
| 19 | 7.1 |
| 20 | 3.0 |
| 21 | 9.7 |
| 22 | 13.0 |
| 23 | 3.0 |
| 24 | 17.0 |
| 25 | 39.8 |
| 26 | 19.9 |
| 27 | 18.5 |
| 28 | 3.6 |
| 29 | 37.8 |
| 30 | 11.7 |
| 31 | 17.7 |
| 32 | 3.2 |
| 33 | 11.3 |
| 34 | 14.1 |
| 35 | 13.5 |
| 36 | 27.4 |
| 37 | 9.6 |

Test Example 2: A measurement test of growth inhibition activity on KRAS-G12D mutant cell line (A-427) (in vitro) A-427 cells (ATCC, Cat #: HTB-53), which are a KRAS-G12D mutant human lung cancer cell line, were suspended in a 10% fetal bovine serum-containing E-MEM medium (manufactured by Fujifilm Wako Pure Chemical Corporation). The cell suspension was seeded into each well of a 384-well U bottom microplate and cultured in an incubator containing 5% CO2 gas at 37° C. for 1 day.

The compound of the present invention was dissolved in DMSO, and the test compound was diluted with DMSO to give a concentration 500 times the final concentration. The solution of the test compound in DMSO was diluted with the medium used for suspending cells and added to each well of the cell-culture plate to give a DMSO final concentration of 0.2%, followed by culture in an incubator containing 5% CO2 gas at 37° C. for another 3 days. The cell count after a 3-day culture in the presence of the compound was measured using CellTiter-Glo 3D Reagent (manufactured by Promega Corporation).

All wells were added with CellTiter-Glo 3D Reagent and mixed for 10 minutes. 30 minutes after mixing, luminescence was measured by a plate reader. The growth inhibition rate was calculated from the following equation, and the concentration of the test compound at which 50% inhibition was achieved (IC50 (μM)) was determined. The following table (Table 3) shows the results.

TABLE 3

| | A427 |
|---|---|
| | CTX |
| Example | IC50(μM) |
| 1 | 1.17 |
| 2 | 0.54 |
| 3 | 0.59 |
| 4 | 1.17 |
| 5 | 1.17 |
| 6 | 0.55 |
| 7 | 0.58 |
| 8 | 0.47 |
| 9 | 0.35 |
| 10 | 6.62 |
| 11 | 7.85 |
| 12 | 8.15 |
| 13 | 8.55 |
| 14 | 1.27 |
| 15 | 1.36 |
| 16 | 2.39 |
| 17 | 1.29 |
| 18 | 0.98 |
| 19 | 1.59 |
| 20 | 1.95 |
| 21 | 0.54 |
| 22 | 4.09 |
| 23 | 1.47 |
| 24 | 1.22 |
| 25 | — |
| 26 | — |
| 27 | 0.65 |
| 28 | 0.82 |
| 29 | 1.26 |
| 30 | 1.38 |
| 31 | 1.95 |
| 32 | 1.08 |
| 33 | 1.27 |
| 34 | 7.14 |
| 35 | 8.84 |
| 36 | >10 |
| 37 | 2.94 |

Growth Inhibition Rate(%)=(C−T)/(C)×100

T: the emission intensity in a well into which a test compound was added.

C: the emission intensity in a well into which a test compound was not added.

The test results reveal that the compound of the present invention has excellent cell growth inhibition activity on KRAS-G12D mutant cell line A-427.

Protein Sequences

Recombinant KRAS G12D (conjugated N-terminus His₆-tag, TEV protease cleavage site and KRAS G12D residues 1-169)

(SEQ ID NO: 1)
MASSHHHHHHSSENLYFQGMTEYKLVVVGADGVGKSALTIQLIQN
HFVDEYDPTIEDSYRKQVVIDGETCLLDILDTAGQEEYSAMRDQY
MRTGEGFLCVFAINNTKSFEDIHHYREQIKRVKDSEDVPMVLVGN
KCDLPSRTVDTKQAQDLARSYGIPFIETSAKTRQGVDDAFYTLVR
EIRKHKEK

Cleaved recombinant SOS1 (residues 564-1049)

(SEQ ID NO: 2)
GEEQMRLPSADVYRFAEPDSEENIIFEENMQPKAGIPIIKAGTVI
KLIERLTYHMYADPNFVRTFLTTYRSFCKPQELLSLIIERFEIPE
PEPTEADRIAIENGDQPLSAELKRFRKEYIQPVQLRVLNVCRHWV
EHHFYDFERDAYLLQRMEEFIGTVRGKAMKKWVESITKIIQRKKI
ARDNGPGHNITFQSSPPTVEWHISRPGHIETFDLLTLHPIEIARQ
LTLLESDLYRAVQPSELVGSVWTKEDKEINSPNLLKMIRHTTNLT
LWFEKCIVETENLEERVAVVSRIIEILQVFQELNNFNGVLEVVSA
MNSSPVYRLDHTFEQIPSRQKKILEEAHELSEDHYKKYLAKLRSI
NPPCVPFFGIYLTNILKTEEGNPEVLKRHGKELINFSKRRKVAEI
TGEIQQYQNQPYCLRVESDIKRFFENLNPMGNSMEKEFTDYLFNK
SLEIEPRNPKPLPRFPKKYSYPLKSPGVRPSNPRPGT

DNA Sequences

For expressing recombinant KRAS G12D (conjugated N-terminus His₆-tag, TEV protease cleavage site and KRAS G12D residues 1-169)

(SEQ ID NO: 3)
ATGGCAAGCAGCCATCATCATCATCATCATAGCAGCGAAAACCTG
TATTTTCAGGGCATGACCGAATATAAACTGGTTGTTGTTGGTGCA
GATGGTGTTGGTAAAAGCGCACTGACCATTCAGCTGATTCAGAAT
CATTTTGTGGATGAGTATGATCCGACCATCGAAGATAGCTATCGT
AAACAGGTTGTGATTGATGGTGAAACCTGTCTGCTGGATATTCTG
GATACCGCAGGTCAAGAGGAATATAGCGCAATGCGTGATCAGTAT
ATGCGTACCGGTGAAGGTTTTCTGTGTGTTTTTGCAATCAACAAT
ACCAAAAGCTTCGAGGATATCCATCATTATCGCGAGCAGATTAAA
CGTGTGAAAGATAGCGAAGATGTTCCGATGGTTCTGGTTGGTAAT
AAATGTGATCTGCCGAGCCGTACCGTTGATACCAAACAGGCACAG
GATCTGGCACGTAGCTATGGTATTCCGTTTATTGAAACCAGCGCA
AAAACCCGTCAGGGTGTTGATGATGCATTTTATACCCTGGTTCGT
GAAATCCGCAAACATAAAGAAAAATGA

For expressing recombinant SOS1 (residues 564-1049 with a conjugated N-terminus His₆-tag and TEV protease cleavage site)

(SEQ ID NO: 4)
ATGGGCAGCAGCCATCATCATCATCATCACAGCAGCGGCCTGGTG
CCGCGCGGCAGCCATATGGCTAGCATGACTGGTGGACAGCAAATG
GGTCGCGGATCCGAAAACCTGTATTTTCAGGGCGAGGAGCAGATG
AGGCTGCCTAGTGCTGATGTTTATAGATTTGCAGAGCCTGACTCT
GAAGAGAATATTATATTTGAAGAGAACATGCAGCCCAAGGCTGGA
ATTCCAATTATCAAAGCAGGAACTGTTATTAAACTTATAGAGAGG
CTTACGTACCATATGTACGCAGATCCCAATTTTGTTCGGACATTT
CTTACAACATACAGATCCTTTTGCAAACCTCAAGAACTACTGAGT
CTTATAATAGAAAGGTTTGAAATTCCAGAGCCTGAGCCAACAGAA
GCTGATCGCATAGCTATAGAGAATGGAGATCAACCCTTGAGTGCA
GAACTGAAAAGATTTAGAAAAGAATATATACAGCCTGTGCAACTG
CGAGTATTAAATGTATGTCGGCACTGGGTAGAGCACCACTTCTAT
GATTTTGAAAGAGATGCATATCTTTTGCAACGAATGGAAGAATTT
ATTGGAACAGTAAGAGGTAAAGCAATGAAAAAATGGGTTGAATCC
ATCACTAAAATAATCCAAAGGAAAAAAATTGCAAGAGACAATGGA
CCAGGTCATAATATTACATTTCAGAGTTCACCTCCCACAGTTGAG
TGGCATATAAGCAGACCTGGGCACATAGAGACTTTTGACCTGCTC
ACCTTACACCCAATAGAAATTGCTCGACAACTCACTTTACTTGAA
TCAGATCTATACCGAGCTGTACAGCCATCAGAATTAGTTGGAAGT
GTGTGGACAAAAGAAGACAAAGAAATTAACTCTCCTAATCTTCTG
AAAATGATTCGACATACCACCAACCTCACTCTGTGGTTTGAGAAA
TGTATTGTAGAAACTGAAAATTTAGAAGAAAGAGTAGCTGTGGTG
AGTCGAATTATTGAGATTCTACAAGTCTTTCAAGAGTTGAACAAC
TTTAATGGTGTCCTTGAGGTTGTCAGTGCTATGAATTCATCACCT
GTTTACAGACTAGACCACACATTTGAGCAAATACCAAGTCGCCAG
AAGAAAATTTTAGAAGAAGCTCATGAATTGAGTGAAGATCACTAT
AAGAAATATTTGGCAAAACTCAGGTCTATTAATCCACCATGTGTG
CCTTTCTTTGGAATTTATCTCACTAATATCTTGAAAACAGAAGAA
GGCAACCCTGAGGTCCTAAAAAGACATGGAAAAGAGCTTATAAAC
TTTAGCAAAAGGAGGAAAGTAGCAGAAATAACAGGAGAGATCCAG
CAGTACCAAAATCAGCCTTACTGTTTACGAGTAGAATCAGATATC
AAAAGGTTCTTTGAAAACTTGAATCCGATGGGAAATAGCATGGAG
AAGGAATTTACAGATTATCTTTTCAACAAATCCCTAGAAATAGAA
CCACGAAACCCTAAGCCTCTCCCAAGATTTCCAAAAAAATATAGC
TATCCCCTAAAATCTCCTGGTGTTCGTCCATCAAACCCAAGACCA
GGTACCTAA

1. A compound represented by the formula (1) or a salt thereof: 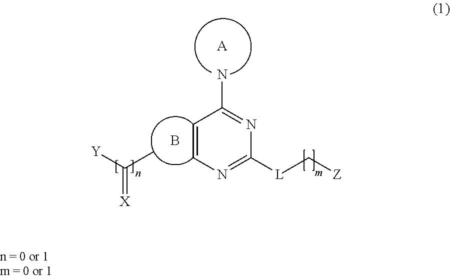
wherein
Ring A represents a substituted or unsubstituted, saturated or unsaturated 8- to 10-membered N-containing bridged ring which contains at least one further heteroatom selected from the group consisting of N, S and O;
Ring B represents a substituted or unsubstituted, 5- to 6-membered saturated or unsaturated ring having at least one heteroatom selected from the group consisting of N, S, and O, a 6-membered aromatic hydrocarbon ring, C3-C6 cycloalkyl ring, C3-C6 cycloalkenyl or an 8- to 10-membered spiro ring, wherein the Ring B is fused with the pyrimidine ring to form a substituted or unsubstituted bicyclic ring;
n is 0 or 1;
X is O orS;
Y represents a substituted or unsubstituted, 6- to 10-membered unsaturated monocyclic or bicyclic ring which contains at least one heteroatom selected from the group consisting of N, S and O, or 6- to 10-membered aromatic hydrocarbon ring;
L represents oxygen atom, or a substituted or unsubstituted C2-C3 alkynyl;
Z represents cyanoalkyl, alkylcarbonylaminoalkyl, alkylaminocarbonyl, alkylaminoalkyl, a substituted or unsubstituted, C3-C6 cycloalkyl, a 5- to 6-membered saturated ring which contains at least one heteroatom selected from the group consisting of N, S and O, or an 8- to 10-membered partially unsaturated ring which contains at least one heteroatom selected from the group consisting of N, S and O;
when L is C2-C3 alkynyl, Z is alkylaminocarbonyl or alkylaminoalkyl;
m is 0 or 1.

2. The compound or salt thereof according to claim 1, wherein the 8- to 10-membered N-containing bridged ring is piperazinyl ring-based, 8-membered N-containing bridged ring, which may be substituted by R1 or R2, and
when the 8-membered N-containing bridged ring is substituted by R1, the R1 is substituted on a nitro atom of piperazinyl ring, and is substituted by R2, the R2 is substituted on any one of carbon atoms of piperazinyl ring; wherein the R1 represents hydrogen atom, or hydroxyl, and the R2 represents a hydrogen atom, halogen atom, alkoxycarbonyl, cyano, or hydroxyalkyl.

3. The compound or salt thereof according to claim 1, wherein the Ring A is represented by any one of the formula (2a) to (2c) which may be substituted by R1 and R2: 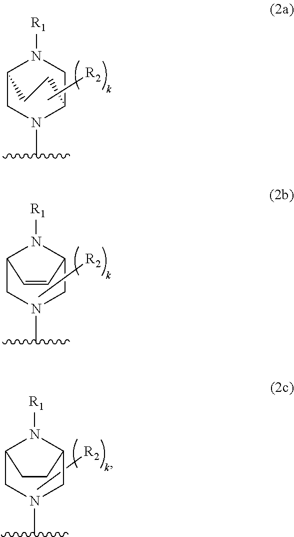
wherein R1 represents hydrogen atom, C1-C6 alkyl, or hydroxyl; R2 represents hydrogen atom, halogen atom, alkoxycarbonyl, cyano, or hydroxyalkyl; and
k is 0 to 6.

4. The compound or salt thereof according to claim 1, wherein the Ring A is represented by the formula (3a) or (3b):

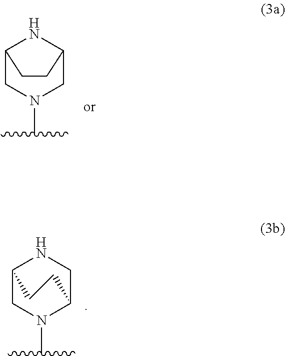 [SMILES: *N1CC2CCC(C1)N2 ; *N1C[C@@H]2CC[C@H]1CN2]

5. The compound or salt thereof according to claim 1,
wherein the Ring B represents:
(i) a 5- to 6-membered saturated or unsaturated ring which contains at least one heteroatom selected from the group consisting of N, S, and O,
(ii) a 6- to 10-membered aromatic hydrocarbon ring,
(iii) C3-C6 cycloalkyl, C3-C6 cyclealkenyl or
(iv) an 8- to 10-membered spiro ring;
wherein the Ring B is fused with the pyrimidine ring to form a substituted or unsubstituted bicyclic ring; and
wherein the Ring B in the bicyclic ring may be substituted by halogen atom, C1-C6 alkyl, alkylcarbonyl or 4- to 6-membered saturated monocyclic ring which contains at least one heteroatom selected from N, S, and O.

6. The compound or salt thereof according to claim 5, wherein the heteroatom in 5- to 6-membered saturated or unsaturated ring is N or O.

7. The compound or salt thereof according to claim 1,
wherein the Ring B represents benzene, piperidine, pyrrolidine, cyclohexane, cyclohexene, tetrahydro-2H-pyran, 3,4-dihydro-2H-pyran, or spiro[2.5]octane;
wherein the Ring B may be substituted by halogen atom, C1-C6 alkyl, alkylcarbonyl or oxetanyl; and
when the Ring B is pyrrolidine, n is 1 and X is O or S, and when the Ring B is not pyrrolidine, n is 0.

8. The compound or salt thereof according to claim 1,
wherein the Ring B represents unsubstituted benzene, piperidine, pyrrolidine, tetrahydro-2H-pyran or 3,4-dihydro-2H-pyran; and
when Ring B is pyrrolidine, n is 1 and X is O or S, and when Ring B is not pyrrolidine, n is 0.

9. The compound or salt thereof according to claim 1,
wherein Y represents an 8- to 10-membered unsaturated bicyclic ring which contains at least one heteroatom selected from the group consisting of N and S, or a 6- to 10-membered aromatic hydrocarbon ring; and
wherein the ring may be substituted by halogen atom, hydroxyl, amino, C1-C6 alkyl, C2-C3 alkenyl, C2-C3 alkynyl or 5- to 6-membered unsaturated monocyclic ring which contains at least one heteroatom selected from the group consisting of N, S and O.

10. The compound or salt thereof according to claim 1,
wherein Y represents benzene, naphthalene, benzo[b]thiophene, thieno[3,2-b]pyridine, isoquinoline, indole, or indazole which may be substituted by halogen atom, hydroxyl, amino, C1-C6 alkyl, C2-C3 alkenyl, C2-C3 alkynyl or thiophenyl.

11. The compound or salt thereof according to claim 1, wherein
L represents oxygen atom, or substituted or unsubstituted C2-C3 alkynyl;
Z represents cyanoalkyl, alkylcarbonylaminoalkyl, alkylaminocarbonyl, alkylaminoalkyl, C3-C6 cycloalkyl, a 5- to 6-membered saturated ring which contains at least one heteroatom selected from the group consisting of N, S, and O, an 8- to 10-membered partially unsaturated ring which contains at least one heteroatoms selected from the group consisting of N, S, and O;
wherein the ring in Z may be substituted by halogen atom, hydroxyl, C1-C6 alkyl, C1-C3 alkoxy, C1-C3 hydroxyalkyl, C1-C3 methoxyalkyl, a substituted or unsubstituted 5- to 6-membered saturated ring which contains at least one heteroatom selected from the group consisting of N, S, and O, and which may be substituted by C1 to C3 alkyl, alkylcarbonylalkyl, hydroxyalkyl, dialkylamino, dialkylaminoalkyl, alkoxyalkyl, or cyanoalkyl;
when L is oxygen atom, m is 0 or 1, and
when L is C2-C3 alkynyl, m is 1 and Z is dimethylaminocarbonyl or dimethylaminomethyl.

12. The compound or salt thereof according to claim 1, wherein
L represents oxygen atom;
m is 0 or 1;
Z represents C3-C6 cycloalkyl, a 5- to 6-membered saturated ring which contains at least one heteroatom selected from the group consisting of N, S, and O, an 8- to 10-membered partially saturated ring which contains at least one heteroatom selected from the group consisting of N, S, and O;
wherein the ring in Z may be substituted by halogen atom, hydroxyl, C1-C6 alkyl, C1-C3 alkoxy, C2-C3 alkynyl, alkylcarbonylalkyl, hydroxyalkyl, dialkylamino, dialkylaminoalkyl, alkoxyalkyl, cyanoalkyl or C1-C6 alkyl which is substituted by 5- to 6-membered saturated ring which contains at least one heteroatom selected from the group consisting of N, S, and O and which may be further substituted by halogen atom.

13. The compound or salt thereof according to claim 1, wherein
L represents oxygen atom;
m is 1;
Z represents C3-C6 cycloalkyl or a 5- to 6-membered saturated ring which contains at least one heteroatom selected from the group consisting of N, S, and, 0;
wherein the ring in Z may be substituted by halogen atom, hydroxyl, cyano, C1-C6 alkyl, C1-C3 alkoxy, alkylcarbonylalkyl, hydroxyalkyl, dialkylamino, dialkylaminoalkyl, alkoxyalkyl, cyanoalkyl, or C1-C6 alkyl which is substituted by 5- to 6-membered saturated ring which contains at least one heteroatom selected from the group consisting of N, S, and O and which may be further substituted by halogen atom.

14. The compound or salt thereof according to claim 1, wherein Z represents:
cyclobutane, cyclopropane, piperidine, morpholine, piperazine, isoindoline or 1,2,3,4-tetrahydroisoquinoline, and which may be substituted by halogen atom, hydroxyl, cyano, C1-C6 alkyl, or C1-C3 alkoxy;
alkylcarbonylalkyl, hydroxyalkyl, dialkylamino, dialkylaminoalkyl, alkoxyalkyl, cyanoalky or C1-C6 alkyl which is substituted by 5- to 6-membered saturated ring which contains at least one heteroatom selected from the group consisting of N and O and which may be further substituted by halogen atom.

15. The compound or salt thereof according to claim 1, wherein Z represents cyclobutane, cyclopropane, piperidine, morpholine, piperazine, isoindoline, or 1,2,3,4-tetrahydroisoquinoline which may be substituted by halogen atom, hydroxyl, C1-C3 alkoxy, methyl, ethyl, isopropanyl, ethylcalbonylmethyl, hydroxyethyl, dimethylamino, dimethylaminomethyl, methoxyethyl, cyanomethyl, morpholylmethyl, or 3-fluoropyrrolidinylmethyl.

16. The compound or salt thereof according to claim 1, wherein
the Ring A is represented by the formula (3a) or (3b): 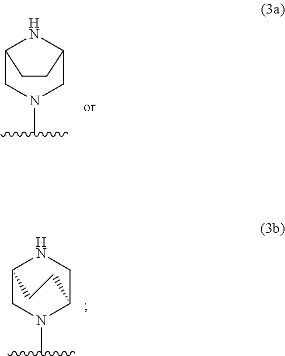 [SMILES: *N1CC2CCC(C1)N2 ; *N1C[C@@H]2CC[C@H]1CN2]
the Ring B represents benzene, piperidine, or pyrrolidine which may be substituted by halogen atom or C1-C6 alkyl;
when Ring B is pyrrolidine, n is 1 and X is 0, and when the Ring B is not pyrrolidine, n is 0;
Y represents naphthalene which may be substituted by halogen atom, hydroxyl, C1-C6 alkyl, C2-C3 alkenyl, or C2-C3 alkynyl;
L represents oxygen atom;
m is 1;
Z represents cyclobutane, cyclopropane, piperidine, morpholine, piperazine, isoindoline, or 1,2,3,4-tetrahydroisoquinoline which is substituted by halogen atom, hydroxyl, C1-C3 alkoxy, methyl, ethyl, isopropanyl, ethylcalbonylmethyl, hydroxyethyl, dimethylamino, dimethylaminomethyl, alkoxyalkyl, cyanomethyl, morpholinylmethyl, or 3-fluoropyrrolidinemethyl.

17. The compound or salt thereof according to claim 1, wherein the compound is selected from the following group of compounds:
(1) 4-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-5-bromonaphthalen-2-ol,
(2) 4-(4-(3,8-diazabicyclo[3.2.1]octan-3-yl)-2-((1-(((R)-3-fluoropyrrolidin-1-yl)methyl)cyclopropyl)methoxy)-5,8-dihydropyrido[3,4-d]pyrimidin-7(6H)-yl)-5-bromonaphthalen-2-ol,
(3) 4-(4-(3,8-diazabicyclo[3.2.1]octan-3-yl)-2-((1-(morpholinomethyl)cyclopropyl)methoxy)-5,8-dihydropyrido[3,4-d]pyrimidin-7(6H)-yl)-5-bromonaphthalen-2-ol,
(4) 4-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-5-methylnaphthalen-2-ol,
(5) 4-(4-(3,8-diazabicyclo[3.2.1]octan-3-yl)-2-((1-(((R)-3-fluoropyrrolidin-1-yl)methyl)cyclopropyl)methoxy)-5,8-dihydropyrido[3,4-d]pyrimidin-7(6H)-yl)-5-methylnaphthalen-2-ol,
(6) 4-(4-(3,8-diazabicyclo[3.2.1]octan-3-yl)-2-((1-(morpholinomethyl)cyclopropyl)methoxy)-5,8-dihydropyrido[3,4-d]pyrimidin-7(6H)-yl)-5-methylnaphthalen-2-ol,
(7) 4-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-5-iodonaphthalen-2-ol,
(8) 4-(4-(3,8-diazabicyclo[3.2.1]octan-3-yl)-2-((1-(((R)-3-fluoropyrrolidin-1-yl)methyl)cyclopropyl)methoxy)-5,8-dihydropyrido[3,4-d]pyrimidin-7(6H)-yl)-5-iodonaphthalen-2-ol,
(9) 4-(4-(3,8-diazabicyclo[3.2.1]octan-3-yl)-2-((1-(morpholinomethyl)cyclopropyl)methoxy)-5,8-dihydropyrido[3,4-d]pyrimidin-7(6H)-yl)-5-iodonaphthalen-2-ol,
(10) (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl)(8-ethynyl-3-hydroxynaphthalen-1-yl)methanone,
(11) (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl)(8-ethynyl-3-hydroxynaphthalen-1-yl)methanone,
(12) (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl)(3-hydroxy-8-iodonaphthalen-1-yl)methanone,
(13) (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl)(3-hydroxy-8-iodonaphthalen-1-yl)methanone,
(14) 4-(4-(3,8-diazabicyclo[3.2.1]octan-3-yl)-6-chloro-2-((1-((dimethylamino)methyl)cyclopropyl)methoxy)-8-fluoroquinazolin-7-yl)naphthalen-2-ol,
(15) 4-(4-(3,8-diazabicyclo[3.2.1]octan-3-yl)-2-((1-((dimethylamino)methyl)cyclopropyl)methoxy)-8-fluoroquinazolin-7-yl)naphthalen-2-ol,
(16) 4-(4-(3,8-diazabicyclo[3.2.1]octan-3-yl)-8-fluoro-2-(((S)-1-methylpyrrolidin-2-yl)methoxy)quinazolin-7-yl)naphthalen-2-ol,
(17) 4-(4-(3,8-diazabicyclo[3.2.1]octan-3-yl)-2-((cis-2-(dimethylamino)cyclobutyl)methoxy)-8-fluoroquinazolin-7-yl)naphthalen-2-ol,
(18) 4-(4-(3,8-diazabicyclo[3.2.1]octan-3-yl)-2-((1-((dimethylamino)methyl)cyclopropyl)methoxy)-6,8-difluoroquinazolin-7-yl)naphthalen-2-ol
(19) 4-(4-(3,8-diazabicyclo[3.2.1]octan-3-yl)-2-((1-((dimethylamino)methyl)cyclopropyl)methoxy)-6-ethyl-8-fluoroquinazolin-7-yl)naphthalen-2-ol,
(20) 4-(4-((1S,4S)-2,5-diazabicyclo[2.2.2]octan-2-yl)-2-((1-((dimethylamino)methyl)cyclopropyl)methoxy)-5,8-dihydropyrido[3,4-d]pyrimidin-7(6H)-yl)-5-bromonaphthalen-2-ol,
(21) 4-(4-(3,8-diazabicyclo[3.2.1]octan-3-yl)-2-((1-((dimethylamino)methyl)-2,2-difluorocyclopropyl)methoxy)-5,8-dihydropyrido[3,4-d]pyrimidin-7(6H)-yl)-5-bromonaphthalen-2-ol,
(22) 1-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-8-bromoisoquinolin-3-amine,
(23) 4-(4-(3,8-diazabicyclo[3.2.1]octan-3-yl)-2-((1-((dimethylamino)methyl)cyclopropyl)methoxy)-7,8-dihydro-5H-pyrano[4,3-d]pyrimidin-7-yl)-5-ethynylnaphthalen-2-ol,
(24) 1-(1-(((4-(3,8-diazabicyclo[3.2.1]octan-3-yl)-7-(8-ethynylnaphthalen-1-yl)-5,6,7,8-tetrahydroquinazolin-2-yl)oxy)methyl)cyclopropyl)-N,N-dimethylmethanamine,
(25) 4-((1-(((4-(3,8-diazabicyclo[3.2.1]octan-3-yl)-7-(8-iodonaphthalen-1-yl)-5,6,7,8-tetrahydropyrido[3,4-d]pyrimidin-2-yl)oxy)methyl)cyclopropyl)methyl)morpholine,
(26) 4-(4-(3,8-diazabicyclo[3.2.1]octan-3-yl)-2-(((R)-1-methylpyrrolidin-2-yl)methoxy)-5,8-dihydropyrido[3,4-d]pyrimidin-7(6H)-yl)-5-bromonaphthalen-2-ol,
(27) 4-(4-(3,8-diazabicyclo[3.2.1]octan-3-yl)-2-(((2S,4R)-4-fluoro-1-methylpyrrolidin-2-yl)methoxy)-5,8-dihydropyrido[3,4-d]pyrimidin-7(6H)-yl)-5-bromonaphthalen-2-ol,
(28) 4-(4-((1S,4S)-2,5-diazabicyclo[2.2.2]octan-2-yl)-2-((1-(morpholinomethyl)cyclopropyl)methoxy)-5,8-dihydropyrido[3,4-d]pyrimidin-7(6H)-yl)-5-bromonaphthalen-2-ol,
(29) 1-(1-(((4-(3,8-diazabicyclo[3.2.1]octan-3-yl)-7-(8-bromonaphthalen-1-yl)-5,6,7,8-tetrahydropyrido[3,4-d]pyrimidin-2-yl)oxy)methyl)-2,2-difluorocyclopropyl)-N,N-dimethylmethanamine,
(30) 4-(4-(3,8-diazabicyclo[3.2.1]octan-3-yl)-2-((1-((dimethylamino)methyl)-2,2-dimethylcyclopropyl)methoxy)-5,8-dihydropyrido[3,4-d]pyrimidin-7(6H)-yl)-5-bromonaphthalen-2-ol,
(31) 4-(4-(3,8-diazabicyclo[3.2.1]octan-3-yl)-2-(((S)-1-methylpyrrolidin-2-yl)methoxy)-5,8-dihydropyrido[3,4-d]pyrimidin-7(6H)-yl)-5-bromonaphthalen-2-ol,
(32) 4-(4-(3,8-diazabicyclo[3.2.1]octan-3-yl)-2-(((2S,4R)-4-methoxy-1-methylpyrrolidin-2-yl)methoxy)-5,8-dihydropyrido[3,4-d]pyrimidin-7(6H)-yl)-5-bromonaphthalen-2-ol,
(33) 4-(4-(3,8-diazabicyclo[3.2.1]octan-3-yl)-2-((1-(morpholinomethyl)cyclopropyl)methoxy)-7,8-dihydro-5H-pyrano[4,3-d]pyrimidin-7-yl)-5-ethynylnaphthalen-2-ol,
(34) (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl)(3-hydroxy-8-vinylnaphthalen-1-yl)methanone,
(35) (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl)(3-hydroxy-8-vinylnaphthalen-1-yl)methanone,
(36) 1-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-8-ethynylisoquinolin-3-amine, and
(37) 4-(4-(3,8-diazabicyclo[3.2.1]octan-3-yl)-2-((1-((dimethylamino)methyl)cyclopropyl)methoxy)-7,8-dihydro-5H-pyrano[4,3-d]pyrimidin-7-yl)-5-bromonaphthalen-2-ol.

18. A pharmaceutical preparation comprising the compound or a salt thereof according to claim 1.

19. A pharmaceutical composition comprising the compound or a salt thereof according to claim 1, and a pharmaceutically acceptable carrier.

20.-27. (canceled)

28. A method for treating a tumor, the method comprising administering an effective amount of the compound or a salt thereof according to claim 1 to a subject in need thereof.

29. A method for treating a tumor, the method comprising administering an antitumor agent to a subject in need thereof in combination with a therapeutically effective amount of one or more other antitumor drugs, the antitumor agent comprising the compound or a salt thereof according to claim 1.

30. The method of claim 28, wherein the tumor is a cancer.

31. The method of claim 30, wherein the cancer is at least one selected from the group consisting of carcinoma, squamous carcinoma, adenocarcinoma, sarcoma, leukemia, neuroma, melanoma, and lymphoma.

32. The method of claim 31, wherein the squamous carcinoma is a cancer of uterine cervix, tarsus, conjunctiva, vagina, lung, oral cavity, skin, bladder, tongue, larynx or esophagus.

33. The method of claim 31, wherein the adenocarcinoma is a cancer of prostate, small intestine, endometrium, uterine cervix, large intestine, lung, pancreas, esophagus, rectum, uterus, stomach, breast or ovary.

34. The method of claim 30, wherein the cancer is lung cancer, pancreatic cancer, rectal cancer, colon cancer, colorectal cancer or uterine cancer.

35.-39. (canceled)
